SEQUENCE LISTING (59 sequences)

SEQ ID NO: 1 - protein, 26 aa, Artificial Sequence
  Synthetic Construct
KAQKAQAKQAKQAQKAQKAQAKQAKQ

SEQ ID NO: 2 - protein, 26 aa, Artificial Sequence
  Synthetic Construct
RAQRAQARQARQAQRAQRAQARQARQ

SEQ ID NO: 3 - protein, 26 aa, Artificial Sequence
  Synthetic Construct
GAQGAQAGQAGQAQGAQGAQAGQAGQ

SEQ ID NO: 4 - protein, 15 aa, Artificial Sequence
  Synthetic Construct
KAKAKAKAKAKAKAK

SEQ ID NO: 5 - protein, 29 aa, Artificial Sequence
  Synthetic Construct
KAQAKAQAKAQAKAQAKAQAKAQAKAQAK

SEQ ID NO: 6 - protein, 26 aa, Artificial Sequence
  Synthetic Construct
KAQQAQAKQAQQAQKAQQAQAKQAQQ

SEQ ID NO: 7 - protein, 26 aa, Artificial Sequence
  Synthetic Construct
QAQKAQAQQAKQAQQAQKAQAQQAKQ

SEQ ID NO: 8 - protein, 26 aa, Artificial Sequence
  Synthetic Construct
KAQKAQAKQAKQAQKAQKAQAKQAKQ

SEQ ID NO: 9 - protein, 26 aa, Artificial Sequence
  Synthetic Construct
KTVKTVTKVTKVTVKTVKTVTKVTKV

SEQ ID NO: 10 - protein, 26 aa, Artificial Sequence
  Synthetic Construct
  VARIANT (1)..(26): D form enantiomer
KAQKAQAKQAKQAQKAQKAQAKQAKQ

SEQ ID NO: 11 - protein, 18 aa, Artificial Sequence
  Synthetic Construct
  VARIANT 2: D form enantiomer
VYKVKTKVKTKVKTKVKT

SEQ ID NO: 12 - protein, 31 aa, Artificial Sequence
  Synthetic Construct
  VARIANT (1)..(3): D form enantiomer
AQAYSKAQKAQAKQAKQAQKAQKAQAKQAKQ

SEQ ID NO: 13 - protein, 31 aa, Artificial Sequence
  Synthetic Construct
  VARIANT (1)..(3): D form enantiomer
AQAYARAQRAQARQARQAQRAQRAQARQARQ

SEQ ID NO: 14 - protein, 31 aa, Artificial Sequence; identical to SEQ ID NO: 12
  Synthetic Construct
  VARIANT (4)..(31): D form enantiomer

SEQ ID NO: 15 - protein, 31 aa, Artificial Sequence; identical to SEQ ID NO: 12
  Synthetic Construct

SEQ ID NO: 16 - protein, 31 aa, Artificial Sequence; identical to SEQ ID NO: 12
  Synthetic Construct
  VARIANT 4: D form enantiomer

SEQ ID NO: 17 - protein, 40 aa, Artificial Sequence
  Synthetic Construct
KAQKAQAKQAKQAQKAQKAQAKQAKQAQKAQKAQAKQAKQ

SEQ ID NO: 18 - protein, 40 aa, Artificial Sequence
  Synthetic Construct
RAQRAQARQARQAQRAQRAQARQARQAQRAQRAQARQARQ

SEQ ID NO: 19 - protein, 111 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKR

SEQ ID NO: 20 - protein, 111 aa, Artificial Sequence
  Synthetic Construct
QVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVS

SEQ ID NO: 21 - protein, 10 aa, Artificial Sequence
  Synthetic Construct
GFSLSSYGVS

SEQ ID NO: 22 - protein, 16 aa, Artificial Sequence
  Synthetic Construct
VIWGDGSTNYHPNLMS

SEQ ID NO: 23 - protein, 3 aa, Artificial Sequence
  Synthetic Construct
LDY

SEQ ID NO: 24 - protein, 16 aa, Artificial Sequence
  Synthetic Construct
RSSQSLVHRNGNTYLH

SEQ ID NO: 25 - protein, 7 aa, Artificial Sequence
  Synthetic Construct
KVSNRFS

SEQ ID NO: 26 - protein, 9 aa, Artificial Sequence
  Synthetic Construct
FQTTYVPNT

SEQ ID NO: 27 - protein, 18 aa, Artificial Sequence
  Synthetic Construct
GGSSRSSSSGGGGSGGGG

SEQ ID NO: 28 - protein, 27 aa, Mus musculus
MASPLTRFLSLNLLLLGESIILGSGEA

SEQ ID NO: 29 - protein, 71 aa, Mus musculus
KVNSTTTKPVLRTPSPVHPTGTSQPQRPEDCRPRGSVKGTGLDFACDIYIWAPLAGICVALLLSLIITLIC

SEQ ID NO: 30 - protein, 34 aa, Mus musculus
AESYENADEELAQPVGRMMDFLSPHGSAWDPSRE

SEQ ID NO: 31 - protein, 67 aa, Mus musculus
GEPQLCYILDAVLFLYGIVLTLLYCRLKIQVRKAAIASREKADAVYTGLNTRSQETYETLKHEKPPQ

SEQ ID NO: 32 - protein, 60 aa, Artificial Sequence
  Synthetic Construct
ISAMQVTPTVGGGYSKAQKAQAKQAKQAQKAQKAQAKQAKQAQKAQKAQAKQAKQVTPTV

SEQ ID NO: 33 - protein, 109 aa, Mus musculus
PNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 34 - protein, 112 aa, Artificial Sequence
  Synthetic Construct
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 35 - protein, 111 aa, Artificial Sequence
  Synthetic Construct
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSS

SEQ ID NO: 36 - protein, 5 aa, Artificial Sequence
  Synthetic Construct
VTPTV

SEQ ID NO: 37 - protein, 169 aa, Artificial Sequence
  Synthetic Construct
ISAMQVTPTVGGGYSKAQKAQAKQAKQAQKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 38 - protein, 28 aa, Artificial Sequence
  Synthetic Construct
MALPVTALLLPLALLLHAARPSQFRVSP

SEQ ID NO: 39 - protein, 38 aa, Artificial Sequence
  Synthetic Construct
TVGGGYSKAQKAQAKQAKQAQKAQKAQAKQAKQVTPTV

SEQ ID NO: 40 - protein, 67 aa, Artificial Sequence
  Synthetic Construct
TTTKPVLRTPSPVHPTGTSQPQRPEDCRPRGSVKGTGLDFACDIYIWAPLAGICVALLLSLIITLIC

SEQ ID NO: 41 - protein, 68 aa, Artificial Sequence
  Synthetic Construct
LGEPQLCYILDAVLFLYGIVLTLLYCRLKIQVRKAAIASREKADAVYTGLNTRSQETYETLKHEKPPQ

SEQ ID NO: 42 - protein, 35 aa, Artificial Sequence
  Synthetic Construct
LYAAPQLHSIQSGPSHEEDADSYENMDKSDDLEPA

SEQ ID NO: 43 - protein, 345 aa, Artificial Sequence
  Synthetic Construct
MALPVTALLLPLALLLHAARPSQFRVSPTVGGGYSKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKTTTKPVLRTPSPVHPTGTSQPQRPEDCRPRGSVKGTGLDFACDIYIWAPLAGICVALLLSLIITLICLGEPQLCYILDAVLFLYGIVLTLLYCRLKIQVRKAAIASREKADAVYTGLNTRSQETYETLKHEKPPQLYAAPQLHSIQSGPSHEEDADSYENMDKSDDLEPA

SEQ ID NO: 44 - protein, 175 aa, Artificial Sequence
  Synthetic Construct
MALPVTALLLPLALLLHAARPSQFRVSPTVGGGYSKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 45 - protein, 86 aa, Artificial Sequence
  Synthetic Construct
MISAVILFLLLLVEQAAALGEPQLCYILDAVLFLYGIVLTLLYCRLKIQVRKAAIASREKADAVYTGLNTRSQETYETLKHEKPPQ

SEQ ID NO: 46 - DNA, 45 bp, Artificial Sequence
  Synthetic Construct
ggtggcggtggctcgggcggtggtgggtcgggtggcggcggatct

SEQ ID NO: 47 - protein, 240 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVS

SEQ ID NO: 48 - protein, 267 aa, Artificial Sequence
  Synthetic Construct
MASPLTRFLSLNLLLLGESIILGSGEADIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVS

SEQ ID NO: 49 - protein, 405 aa, Artificial Sequence
  Synthetic Construct
MASPLTRFLSLNLLLLGESIILGSGEADIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVSKVNSTTTKPVLRTPSPVHPTGTSQPQRPEDCRPRGSVKGTGLDFACDIYIWAPLAGICVALLLSLIITLICGEPQLCYILDAVLFLYGIVLTLLYCRLKIQVRKAAIASREKADAVYTGLNTRSQETYETLKHEKPPQ

SEQ ID NO: 50 - protein, 439 aa, Artificial Sequence
  Synthetic Construct
MASPLTRFLSLNLLLLGESIILGSGEADIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVSKVNSTTTKPVLRTPSPVHPTGTSQPQRPEDCRPRGSVKGTGLDFACDIYIWAPLAGICVALLLSLIITLICGEPQLCYILDAVLFLYGIVLTLLYCRLKIQVRKAAIASREKADAVYTGLNTRSQETYETLKHEKPPQAESYENADEELAQPVGRMMDFLSPHGSAWDPSRE

SEQ ID NO: 51 - protein, 310 aa, Artificial Sequence
  Synthetic Construct
MALPVTALLLPLALLLHAARPSQFRVSPTVGGGYSKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKTTTKPVLRTPSPVHPTGTSQPQRPEDCRPRGSVKGTGLDFACDIYIWAPLAGICVALLLSLIITLICLGEPQLCYILDAVLFLYGIVLTLLYCRLKIQVRKAAIASREKADAVYTGLNTRSQETYETLKHEKPPQ

SEQ ID NO: 52 - protein, 317 aa, Artificial Sequence
  Synthetic Construct
TVGGGYSKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKTTTKPVLRTPSPVHPTGTSQPQRPEDCRPRGSVKGTGLDFACDIYIWAPLAGICVALLLSLIITLICLGEPQLCYILDAVLFLYGIVLTLLYCRLKIQVRKAAIASREKADAVYTGLNTRSQETYETLKHEKPPQLYAAPQLHSIQSGPSHEEDADSYENMDKSDDLEPA

SEQ ID NO: 53 - protein, 282 aa, Artificial Sequence
  Synthetic Construct
TVGGGYSKAQKAQAKQAKQAQKAQKAQAKQAKQVTPTVPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKTTTKPVLRTPSPVHPTGTSQPQRPEDCRPRGSVKGTGLDFACDIYIWAPLAGICVALLLSLIITLICLGEPQLCYILDAVLFLYGIVLTLLYCRLKIQVRKAAIASREKADAVYTGLNTRSQETYETLKHEKPPQ

SEQ ID NO: 54 - protein, 378 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVSKVNSTTTKPVLRTPSPVHPTGTSQPQRPEDCRPRGSVKGTGLDFACDIYIWAPLAGICVALLLSLIITLICGEPQLCYILDAVLFLYGIVLTLLYCRLKIQVRKAAIASREKADAVYTGLNTRSQETYETLKHEKPPQ

SEQ ID NO: 55 - protein, 412 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRFGGGTKLEIKRGGSSRSSSSGGGGSGGGGQVQLKESGPGLVAPSQSLSITCTVSGFSLSSYGVSWVRQPPGKGLEWLGVIWGDGSTNYHPNLMSRSLSISKDISKSQVLFKLNSLQTDDTATYYCVTLDYWGQGTSVTVSKVNSTTTKPVLRTPSPVHPTGTSQPQRPEDCRPRGSVKGTGLDFACDIYIWAPLAGICVALLLSLIITLICGEPQLCYILDAVLFLYGIVLTLLYCRLKIQVRKAAIASREKADAVYTGLNTRSQETYETLKHEKPPQAESYENADEELAQPVGRMMDFLSPHGSAWDPSRE

SEQ ID NO: 56 - protein, 213 aa, Artificial Sequence
  Synthetic Construct
MALPVTALLLPLALLLHAARPSQFRVSPTVGGGYSKAQKAQAKQAKQAQKAQKAQAKQAKQVTPTVAYEEPDSEEGSEFYENDSNLGQDQVSQDGSGYENPEDEPMGPEEEDSFSNAESYENADEELAQPVGRMMDFLSPHGSAWDPSREASSLGSQSYEDMRGILYAAPQLHSIQSGPSHEEDADSYENMDKSDDLEPAWEGEGHMGTWGTT

SEQ ID NO: 57 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 58 - protein, 47 aa, Artificial Sequence
  Synthetic Construct
KVNSTTTKPVLRTPSPVHPTGTSQPQRPEDCRPRGSVKGTGLDFACD

SEQ ID NO: 59 - protein, 24 aa, Artificial Sequence
  Synthetic Construct
IYIWAPLAGICVALLLSLIITLIC

(19) United States
(12) Patent Application Publication
(10) Pub. No.: US 2023/0068507 A1
(43) Pub. Date: Mar. 2, 2023

(54) CHIMERIC ANTIGEN RECEPTORS FOR REMOVAL OF AMYLOID

(71) Applicant: University of Tennessee Research Foundation, Knoxville, TN (US)

(72) Inventor: Jonathan S. WALL, Knoxville, TN (US)

(21) Appl. No.: 17/793,355

(22) PCT Filed: Jan. 15, 2021
(86) PCT No.: PCT/US2021/013727

§ 371 Date: Jul. 15, 2022

Related U.S. Application Data
(60) Provisional application No. 62/962,763, filed on Jan. 17, 2020.

(51) Int. Cl. C07K 16/18; C07K 14/725; C07K 14/705; A61P 25/28

(52) U.S. Cl. CPC C07K 16/18; C07K 14/7051; C07K 14/70517; C07K 14/70503; A61P 25/28; C07K 2319/02; C07K 2319/03; C07K 2317/622; C07K 2317/24; A61K 38/00

(57) ABSTRACT

Provided herein are chimeric receptors comprising amyloid binding regions, as well as cells comprising the chimeric receptors. Also provided herein are methods of treating amyloid-based diseases by administering a cell comprising a chimeric receptor.

### CROSS-REFERENCE TO RELATED APPLICATIONS

This application claims priority to U.S. Provisional Application No. 62/962,763, filed on Jan. 17, 2020, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165992000540SEQLIST.TXT, date recorded: Jan. 15, 2021, size: 54 KB).

### FIELD OF THE INVENTION

This application relates to chimeric antigen receptors that can be used to remove amyloid or treat amyloid-related diseases.

### BACKGROUND

Amyloidosis is a devastating pathology that is associated not only with the development of Alzheimer's disease, but also with lesser known, but similarly devastating, disorders such as immunoglobulin light chain-associated (AL) amyloidosis (Dispenzieri. A., et al., Blood Rev, 2012. 26(4): p. 137-54; Merlini, G., Hematology Am Soc Hematol Edu Program, 2017. 2017(1): p. 1-12). Patients with AL develop amyloid in the heart, liver, spleen, kidneys, and peripheral nerves which leads to organ dysfunction and is invariably fatal. The amyloid deposits in systemic diseases are immunologically inert—they are not recognized or cleared by phagocytic cells of the immune system (macrophages, “Mφ”) and do not illicit an antibody response. In patients presenting with significant cardiac amyloidosis, the prognosis is poor with a median survival of ˜9 mos (Gertz, M. A., et al., Blood, 1991. 77(2): p. 257-62; Grogan, M., A. et al., Heart, 2017. 103(14): p. 1065-1072). Treatment of AL amyloidosis generally involves anti-plasma cell chemotherapy and immunotherapy to suppress plasma cell secretion of the amyloid forming light chain protein. However, clearance of existing tissue amyloid has now become a major goal of many of the novel therapeutics being developed for these patients.

In addition, approximately 3% of the general US population over the age of 50 have a plasma cell disorder known as monoclonal gammopathy of unknown significance (MGUS)(Weiss, B. M., et al., Blood, 2009. 113(22): p. 5418-22). This disorder, when accompanied by secretion of monoclonal immunoglobulin light chain (LC) by the plasma cells, is an ominous precursor to a devastating and often fatal condition, LC-associated (AL) amyloidosis, in which highly ordered protein fibrils composed of LC, or their fragments, deposit in the extracellular space of organs and tissues including the liver, heart, kidneys, spleen, intestines, and nerves (Dispenzieri, A., et al., Blood Rev, 2012. 26(4): p. 137-54; Merlini, G., Hematology Am Soc Hematol Edu Program, 2017. 2017(1): p. 1-12; Wechalekar, A. D., et al., Systemic amyloidosis. Lancet, 2015). The amyloid fibrils deposit in association with heparan sulfate proteoglycans and serum-derived proteins, such as serum amyloid P component (SAP), resulting in a complex pathologic matrix. Although amyloid is an “unnatural” protein aggregate, it is non-immunogenic and surprisingly resistant, in patients, to clearance by phagocytic cells of the innate immune system. In fact, evaluation of autopsy-derived material shows no definitive influx of immune cells.

Despite decades of research into the pathogenesis of AL amyloidosis, and improvements in patient survival rates (Dispenzieri, A., et al., Blood Rev, 2012. 26(4): p. 137-54), the disease remains invariably fatal. The overall survival for subjects presenting with severe cardiac AL-associated amyloidosis is ˜9 months (Grogan, M., A. et al., Heart, 2017. 103(14): p. 1065-1072). This is due to the fact that patients generally present with significant organ-compromising loads of tissue amyloid and an accompanying poor prognosis, particularly when cardiac (Kristen, A. V., et al., J Am Coll Cardiol, 2016. 68(1): p. 13-24) or renal (Kuroda, T., et al., BMC Nephrol, 2012. 13: p. 118) AL amyloidosis are the primary manifestations (Kristen, A. V., et al., J Am Coll Cardiol, 2016. 68(1): p. 13-24; Banypersad, S. M., et al., Eur Heart J, 2015. 36(4): p. 244-51). Established clinical management of patients with AL amyloidosis aims to prevent production of the pro-amyloidogenic precursor LC protein, thereby preventing expansion of the amyloid load. This is accomplished by using plasma cell chemo- and immunotherapy (Chaulagain, C. P. and R. L. Comenzo, Clin Adv Hematol Oncol, 2015. 13(5): p. 315-24; Comenzo, R. L., et al., N Engl J Med, 2008. 358(1): p. 92; author reply 92-3; Sanchorawala, V., et al., Bone Marrow Transplant, 2004. 33(4): p. 381-8; Varga, C. and R. L. Comenzo, Bone Marrow Transplant, 2018), proteasome inhibitors (Chaulagain, C. P. and R. L. Comenzo, Clin Adv Hematol Oncol, 2015. 13(5): p. 315-24; Sidiqi, M. H. and M. A. Gertz, Leuk Lymphoma, 2018: p. 1-7; Milani, P., et al., Kidney Int Rep, 2018. 3(3): p. 530-541), and autologous stem cell transplantation (D'Souza, A., et al., J Clin Oncol, 2015. 33(32): p. 3741-9). However, these approaches are not designed to facilitate active dissolution of existing tissue amyloid. Despite successful hematologic remission in response to these approaches, in the majority of patients, LC protein returns and the disease progresses. In these patients, the incessant accumulation of amyloid in tissues is invariably the major factor contributing to organ dysfunction, worsening quality of life, and mortality.

Uptake of AL amyloid has been demonstrated using at least two amyloid-reactive binding proteins: (1) the mAb 11-1F4 (Hrncic, R., et al., Am J Pathol, 2000. 157(4): p. 1239-46; Solomon, A., et al., Clin Cancer Res, 2003. 9(10 Pt 2): p. 3831S-8S; Solomon, A., et al., Cancer Biother Radiopharm, 2003. 18(6): p. 853-60; Wall, J. S., et al., Blood, 2010. 116(13): p. 2241-4) and (2) peptide p5+14 (Kennel, S. J., et al., Mol Imaging Biol, 2016. 18(4): p. 483-9; Martin. E. B., et al., Sci Rep, 2016. 6: p. 22695; Wall, J. S., et al., Molecules, 2015. 20(5): p. 7657-82). Further, to address the need for “amyloid-clearing” therapeutics, amyloid-reactive monoclonal antibodies (mAbs) have been developed over the last 20 years and, recently, clinical trials of three reagents have been conducted (Richards, D. B., et al., Sci Transl Med, 2018. 10(422); Edwards, C. V., et al., Amyloid, 2017. 24(supl): p. 58-59; Gertz, M. A., et al., J Clin Oncol, 2016. 34(10): p. 1097-103; Gertz, M. A., et al., Am J Hematol, 2016. 91(12): p. E506-E508; Wall, J. S., et al., Blood, 2010. 116(13): p. 2241-4). The mode of action proposed for passive immunotherapy with these mAbs involves specific amyloid binding and opsonization of the amyloid, resulting in localized macrophage (Mφ) activation and phagocytosis of the amyloid (Wall, J. S., et al., Proc Natl Acad Sci USA, 2018. 115(46): p. E10839-E10848). Furthermore, stimulation of the Mφ is mediated through interactions of the mAb Fc domain with Fc-receptors (FcR) or through complement C3 receptors following complement fixation by the amyloid-bound mAb (Bodin, K., et al., Nature, 2010. 468(7320): p. 93-7; Milde, R., et al., Cell Rep, 2015. 13(9): p. 1937-48). Two of the three mAbs that have been clinically evaluated, the chimeric (c) 11-1F4 and humanized NEOD001 mAbs, were generated or underwent preclinical development (Hrncic, R., et al., Am J Pathol, 2000. 157(4): p. 1239-46; Wall, J. S., et al., PLoS One, 2012. 7(12): p. e52686; Solomon, A., D. T. Weiss, and J. S. Wall, Clin Cancer Res, 2003. 9(10 Pt 2): p. 3831S-8S; O'Nuallain, B., et al. Amyloid and Amyloidosis: Proceedings of the Xth International Symposium on Amyloidosis. 2005. Tours, France: CRC Press). Despite positive clinical data in Phase 1 trials, the NEOD001 program was terminated due to a lack of efficacy in interim evaluation of the pivotal Phase 3 trial (NCT02312206), and the c11-1F4 Phase 2 trial has stalled. The Phase 1 trial of the third mAb (Glaxo-SmithKline; NCT03044353) generated data that indicated that treatment of AL patients with dezamizumab could result in a dramatic and measureable clearance of tissue amyloid (Richards, D. B., et al., Sci Transl Med, 2018. 10(422)). This trial has recently been halted with data to be announced. Accordingly, the development of further “amyloid-clearing” therapeutics is required. Notwithstanding the overall results for these mAb trials, data were generated that support the hypothesis that opsonization of AL amyloid can result in its dissolution.

One approach to clearing a target of interest is to administer phagocytic cells expressing chimeric antigen receptors (CAR) specific to the target. For example, Mφ presenting a chimeric antigen receptor (CAR) comprising a CD19 binding receptor and cytoplasmic pro-phagocytosis signaling elements has been demonstrated to exhibit enhanced uptake of CD19-coated beads and improved killing of CD19-expressing B-lymphocytes in culture (Morrissey, M. A., et al., Elife, 2018. 7). Specifically, CARs using cytoplasmic elements that contained phagocytosis-signaling immunoreceptor, tyrosine-based activation motifs (ITAMs; see Hamerman, J. A., et al., Immunol Rev, 2009. 232(1): p. 42-58; Swisher, J. F. and G. M. Feldman, Immunol Rev, 2015. 268(1): p. 160-74) significantly enhanced uptake of CD19 and CD22-coated particles up to 20 μm in diameter. Additionally, killing of CD19-positive Raji B lymphocytes was observed in culture. This study focused on binding tumor cell antigens and tumor cell-killing, and also demonstrated that several unique CAR constructs enhanced phagocytosis, while showing that some were more efficient than others.

Taken together, there exists a need in the art for therapeutics designed to clear amyloid deposits.

### SUMMARY OF THE INVENTION

Provided herein is a chimeric receptor comprising: a cytoplasmic domain, wherein the cytoplasmic domain comprises a signaling domain of a receptor that when activated activates a macrophage; a transmembrane domain; and an extracellular domain, wherein the extracellular domain comprises an amyloid binding region.

In some embodiments, wherein the extracellular domain comprises an antibody or functional fragment thereof. In some embodiments, the antibody fragment is an scFv. In some embodiments, the antibody comprises a VL comprising a CDRL1, a CDRL2, and an CDRL3 and a VH comprising a CDRH1, a CDRH2, and a CDRH3, wherein the CDRL1 comprises the amino acid sequence set forth in SEQ ID NO:24; the CDRL2 comprises the amino acid sequence set forth in SEQ ID NO:25; the CDRL3 comprises the amino acid sequence set forth in SEQ ID NO:26; the CDRH1 comprises the amino acid sequence set forth in SEQ ID NO:21; the CDRH2 comprises the amino acid sequence set forth in SEQ ID NO:22; and the CDRH3 comprises the amino acid sequence set forth in SEQ ID NO:23. In some embodiments, the antibody comprises a VL comprising the amino acid sequence set forth in SEQ ID NO:19 or 34 and a VH comprising the amino acid sequence set forth in SEQ ID NO:20 or 35. In some embodiments, the amyloid binding region comprises an 11-1F4 antibody fragment. In some embodiments, the antibody fragment is humanized.

In some embodiments, the extracellular domain comprises an amyloid-reactive peptide. In some embodiments, the amyloid-reactive peptide comprises the sequence set forth in SEQ ID NO:1-18.

In some embodiments, the amyloid binding region is joined directly or indirectly to a CH2 domain or fragment thereof. In some embodiments, the CH2 domain comprises an amino acid sequence having at least 80, 85, 90, 95, 97, 98, or 99% sequence identity to the amino acid sequence set forth in SEQ ID NO:33.

In some embodiments, the cytoplasmic domain comprises a cytoplasmic domain I, cytoplasmic domain II, or functional fragment thereof. In some embodiments, the cytoplasmic domain comprises an amino acid sequence having at least 80, 85, 90, 95, 97, 98, or 99 sequence identity to the amino acid sequence set forth in SEQ ID NO: 30, 31, 41, 42, or 45.

In some embodiments, binding of an amyloid to the extracellular domain activates the cytoplasmic domain of the chimeric receptor.

In some embodiments, the chimeric the receptor has 80, 85, 90, 95, 97, 98, or 99% sequence identity to the sequence set forth in SEQ ID NO: 43 with or without the secretory leader sequence. In some embodiments, each component of the receptor has 80, 85, 90, 95, 97, 98, or 99% sequence identity to the corresponding component of SEQ ID NO:43 together or separately.

Also provided herein is nucleic acid encoding the chimeric receptor provided herein.

In some embodiments provided herein is a an engineered cell comprising nucleic acid encoding a chimeric receptor provided herein.

In some embodiments, provided herein is a method for removing an amyloid, comprising contacting an amyloid deposit with a chimeric receptor provided herein or an engineered cell of claim comprising a chimeric receptor provided herein. In some embodiments, the amyloid is AA, AL, AK ATTR, Aß2M, Wild type TTR, AApoAI, AApoAII, AGel, ALys, ALect2, Afib, ACys, ACal, AMedin, AIAPP, APro, AIns, APrP, or Aβ.

In some embodiments, the amyloid binding region of the chimeric receptor has binding affinity to the amyloid.

In some embodiments, contacting the amyloid deposit with the chimeric receptor results in at least partial clearance of the amyloid.

Also provided herein is a method of treating a subject having an amyloid disorder comprising administering to the subject a chimeric receptor provided herein or an engineered cell provided herein. In some embodiments, administering to the subject the chimeric receptor comprises administering a macrophage or monocyte expressing the chimeric receptor.

### BRIEF DESCRIPTION OF THE DRAWINGS

FIG. 1 shows provides a schematic representation of 11-1F4 scFv (left, labeled “(i) 11-1F4”) and p5+14 peptide-based (right, labeled “(ii) Peptide”) amyloid-specific CAR structures. At left, the 11-1F4-based CAR includes, from N- to C-terminus, an extracellular domain consisting of an 11-1F4 antibody scFv (including a VL (represented as a dark gray oval), a scFv linker (line connecting the VL and the VH), and a VH (medium gray oval), a spacer sequence (diamond), a transmembrane domain (rectangle), and a cytoplasmic domain (larger medium gray oval). At right, the peptide-based CAR includes, from N- to C-terminus, an extracellular domain consisting of a p5+14 peptide, a first spacer sequence (smaller diamond), a CH2 domain (checkerboard oval), a second spacer sequence (larger diamond), a transmembrane domain (rectangle), and a cytoplasmic domain (larger medium gray oval).

FIG. 2 shows whole-body anterior ¹²³I-SAP scintigraphy immediately before monoclonal antibody infusion (left) and at day 42 after monoclonal antibody infusion (right). A marked reduction in hepatic amyloid load was observed. From Richards et al. (Sci Transl Med, 2018 10 (422)).

FIG. 3 shows a schematic representation of the features of a chimeric antigen receptor.

FIGS. 4A-4D show the results of experiments evaluating in vivo administration of m11-1F4. FIG. 4A shows human AL amyloid extract implanted subcutaneously in a mouse (arrow) detected by SPECT/CT image using ¹²⁵I-m11-1F4. FIG. 4B shows that treatment of mice bearing subcutaneous human AL amyloid (arrow) with m11-1F4 caused regression of the lesion. FIG. 4C shows a control treated animal. FIG. 4D shows the biodistribution of ¹²⁴I-m11-1F4 in patient with AL amyloidosis by PET/CT imaging. Arrow is uptake of mAb in enlarged, amyloid-laden liver.

FIGS. 5A-5B show results showing that peptide p5+14 binds human AL amyloid in tissue sections. FIG. 4A shows that when radioiodinated, ¹²⁴I-p5+14 was seen to co-localize with organs likely to contain amyloid (kidneys, spleen, and pancreas) in a patient with AL amyloidosis. FIG. 5B shows PET/CT imaging of the patient.

FIG. 6 shows a schematic representation of proposed 11-1F4 scFv (let, labeled “(i) 1-1F4”) and p5+14 peptide-based (right, labeled “(ii) Peptide”) CAR structures.

### DETAILED DESCRIPTION

### I. Definitions

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710) and other similar references. As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

Ranges can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect. In certain example embodiments, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary,” or “exemplified,” are not meant to show preference, but rather to explain that the aspect discussed thereafter is merely one example of the aspect presented.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. In case of conflict, the present specification, including explanations of terms, will control. In addition, the materials, methods, and examples are illustrative only and not intended to be limiting.

To facilitate review of the various embodiments of this disclosure, the following explanations of specific terms are provided:

Administration: The introduction of a composition into a subject by a chosen route. For example, if the chosen route is intravenous, the composition is administered by introducing the composition into a vein of the subject. In some examples a peptides are administered to a subject.

The terms amyloids, amyloid deposits, amyloid fibrils, and amyloid fibers refer to insoluble fibrous protein aggregates sharing specific structural traits. The protein aggregates have a tertiary structure, for example, that is formed by aggregation of any of several different proteins and that consists of an ordered arrangement of β sheets stacked perpendicular to a fiber axis. See Sunde et al., J. Mol. Biol. (1997) 273:729-39. Abnormal accumulation of amyloids in organs may lead to amyloidosis Although they are diverse in their occurrence, all amyloids have common morphologic properties in that they stain with specific dyes such as Congo red and have a characteristic red-green birefringent appearance in polarized light after staining. Amyloids also share common ultrastructural features and common x-ray diffraction and infrared spectra.

Amyloidosis refers to a pathological condition or disease characterized by the presence of amyloids, such as the presence of amyloid deposits. “Amyloid diseases” or “amyloidosis” are diseases associated with the formation, deposition, accumulation or persistence of amyloid fibrils. Such diseases include, but are not limited to, Alzheimer's disease, Down's syndrome, hereditary cerebral hemorrhage with amyloidosis of the Dutch type, and cerebral beta-amyloid angiopathy. Other amyloid diseases such as systemic AA amyloidosis, AL amyloidosis, ATTR amyloidosis, ALect2 amyloidosis, and IAPP amyloidosis of type II diabetes are also amyloid diseases.

Amyloidogenic refers to producing or tending to produce amyloid deposits. For example, certain soluble monomeric proteins can undergo extensive conformational changes leading to their aggregation into well-ordered, unbranching, 8- to 10-nm wide fibrils, which culminate in the formation of amyloid aggregates. More than thirty proteins, for example, have been found to form amyloid deposits (or amyloids) in man. Not all proteins within the class of diverse proteins, such as immunoglobulin light chains, are capable of forming amyloid, i.e., some proteins are non-amyloidogenic, meaning that they do not tend to form amyloids. Other proteins of the class, however, can form amyloid deposits and are thus amyloidogenic. Furthermore, within the class of light chain protein, some may be deemed more “amyloidogenic” than others based upon the ease with which they form amyloid fibrils. Certain light chain proteins are deemed non-amyloidogenic or less amyloidogenic because of their inability to readily form amyloid fibrils in patients or in vitro.

Animal: Living multi-cellular vertebrate organisms, a category that includes, for example, mammals and birds. The term mammal includes both human and non-human mammals. Similarly, the term “subject” includes both human and veterinary subjects. In some examples a subject is a subject, such as a subject suffering from an amyloid disease.

Clearance: The terms “clear” or “clearance” refer to reducing or removing by a measurable degree. For example, the clearance of an amyloid deposit as described herein relates to reducing or removing the deposit to a measurable or discernable degree. Clearance may result in 100% removal, but is not required to. Rather, clearance may result in less than 100% removal, such as about 10%, 20%, 30%, 40%, 50%, 60% or more removal.

Antibody refers to single chain, two-chain, and multi-chain proteins and glycoproteins belonging to the classes of polyclonal, monoclonal, chimeric and hetero immunoglobulins (monoclonal antibodies being preferred); it also includes synthetic and genetically engineered variants of these immunoglobulins. An “antibody fragment” includes Fab, Fab′, F(ab)2, scFv and Fv fragments, as well as any portion of an antibody having specificity toward a desired target epitope or epitopes. A “monoclonal antibody” is an antibody produced by a single done of B-lymphocytes. Monoclonal antibodies are produced by methods known to those of skill in the art, for instance by making hybrid antibody-forming cells from a fusion of myeloma cells with immune spleen cells.

An epitope refers to a site on an antigen recognized by an antibody, as determined by the specificity of the antibody amino acid sequence Epitopes are also called antigenic determinants. For example, the epitope may be portion of a recombinant protein that is recognized by the particular antibody. Further, the epitope may be a conformational epitope and linear epitope.

Chimeric antibody refers to an antibody that includes sequences derived from two different antibodies, which typically are of different species. Most typically, chimeric antibodies include human and murine antibody fragments, generally human constant and murine variable regions.

Humanized antibody refers to an antibody derived from a non-human antibody, typically murine, and a human antibody which retains or substantially retains the antigen-binding properties of the parent antibody but which is less immunogenic in humans.

Complementarity Determining Region or CDR refers to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. By definition, the CDRs of the light chain are bounded by the residues at positions 24 and 34 (L-CDR1), 50 and 56 (L-CDR2), 89 and 97 (L-CDR3); the CDRs of the heavy chain are bounded by the residues at positions 31 and 35b (H-CDR1), 50 and 65 (H-CDR2), 95 and 102 (H-CDR3), using the numbering convention delineated by Kabat et al., (1991) Sequences of Proteins of Immunological Interest, 5th Edition, Department of Health and Human Services, Public Health Service. National Institutes of Health, Bethesda (NIH Publication No. 91-3242).

Framework region refers to amino acid sequences interposed between CDRs. These portions of the antibody serve to hold the CDRs in an appropriate orientation for antigen binding.

Effective amount or Therapeutically effective amount: The amount of agent that is sufficient to prevent, treat (including prophylaxis), reduce and/or ameliorate the symptoms and/or underlying causes of any of a disorder or disease, for example to prevent, inhibit, and/or amyloidosis. In some embodiments, an “effective amount” is sufficient to reduce or eliminate a symptom of a disease. An effective amount can be administered one or more times.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et ah, Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Inhibit: To reduce by a measurable degree. Inhibition does not, for example, require complete loss of function or complete cessation of the aspect being measured. For example, inhibiting plaque formation can mean stopping further growth of the plaque, slowing further growth of the plaque, or reducing the size of the plaque.

Inhibiting or treating a disease: Inhibiting the full development of a disease or condition, for example, inhibiting amyloidosis. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

With regard to amyloid deposit formation, “inhibition” refers to the prevention of reduction in the formation of the amyloid deposit, such as when compared to a control. For example, inhibition may result in a reduction of about 10%, 20%, 30%, 40%, 50%, 60% or more of an amyloid deposit as compared to a control.

Isolated: An “isolated” biological component, such as a peptide, cell, nucleic acid, or serum samples has been substantially separated, produced apart from, or purified away from other biological components in the cell of the organism in which the component naturally occurs, for instance, other chromosomal and extrachromosomal DNA and RNA, and proteins Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity, rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Join: As used herein, the term “join,” “joined,” “link,” or “linked” refers to any method known in the art for functionally connecting proteins and/or protein domains. For example, one protein domain may be linked to another protein domain via a covalent bond, such as in a recombinant fusion protein, with or without intervening sequences or domains. Joined also includes, for example, the integration of two sequences together, such as placing two nucleic acid sequences together in the same nucleic acid strand so that the sequences are expressed together.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

cDNA refers to a DNA that is complementary or identical to an mRNA, in either single stranded or double stranded form.

Encoding refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Recombinant nucleic acid refers to a nucleic acid having nucleotide sequences that are not naturally joined together. This includes nucleic acid vectors, such as adenoviral vectors, comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well. A first sequence is an “antisense” with respect to a second sequence if a polynucleotide whose sequence is the first sequence specifically hybridizes with a polynucleotide whose sequence is the second sequence.

Pharmaceutically acceptable carriers: The pharmaceutically acceptable carriers of use are conventional. Remington's Pharmaceutical Sciences, by E. W. Martin, Mack Publishing Co., Easton, Pa., 19^{th} Edition (1995), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. In some examples, a peptide is one or more of the peptides disclosed herein.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sequence Identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2: 482, 1981; Needleman & Wunsch J. Mol. Bio. 48:443, 1970; Pearson & Lipman Proc. Natl. Acad. Sci. USA 85: 2444. 1988; Higgins & Sharp Gene 73: 237-244, 1988; Higgins & Sharp CABIOS 5: 151-153, 1989; Corpet et al., Nuc. Acid Res. 16, 10881-90, 1988; Huang et al. Computer Appl. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to a subject or a cell.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses.

A subject refers to a vertebrate. The vertebrate may be a mammal, for example, a human. The subject may be a human patient A subject may be a patient suffering from or suspected of suffering from a disease or condition and may be in need of treatment or diagnosis or may be in need of monitoring for the progression of the disease or condition. The patient may also be in on a treatment therapy that needs to be monitored for efficacy. In some example embodiments, a subject includes a subject suffering from amyloidosis, such as Alzheimer's, Huntington's or prion diseases, or peripheral amyloidosis such as seen in patients with light chain (AL) amyloidosis and type 2 diabetes.

The terms treating or treatment refer to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Preferably, residue positions which are not identical differ by conservative amino acid substitutions. The term “conservative amino aid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of the antigen receptors are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3)non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. (Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

“Framework” or “FR” refers to variable domain residues other than CDR residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3(L3)-FR4; or FR1-CDR-H1(L1)-FR2-CDR-H2(L2)-FR3-CDR3-H3(L3)-FR4.

### II. Chimeric Receptors that Bind Amyloid

In light of the inadequate treatment options currently available for patients with AL amyloidosis, there is an urgent clinical need for alternative approaches for effectively removing tissue amyloid. Accordingly, the present disclosure is based in part on the design of constructs for chimeric antigen receptor phagocytic (CAR-P) macrophages (“Mφ”) (Morrissey, M. A., et al., Elife, 2018. 7), modelled after the CAR-T-lymphocyte anti-tumor technology. In some embodiments, chimeric antigen receptors are modular, synthetic, single chain proteins that comprise three functional regions: (i) the binding receptor (extracellular domain); (ii) the spacer and transmembrane region, and; (iii) the cytoplasmic signaling domain (intracellular)(Zhang, C., et al., Biomark Res, 2017. 5: p. 22). In some embodiments, a cleavable leader, or signal peptide, is placed at the N-terminal of the protein to direct passage through the endoplasmic reticulum and promote display on the plasma membrane (see. e.g., FIG. 3). Each “module” may be derived from proteins to achieve specific target binding and the desired cellular response, elicited through the cytoplasmic signaling domain, e.g., the CD3ζ (domain (Daniyan, A. F. and R. J. Brentjens, J Leukoc Biol, 2016. 100(6): p. 1255-1264; Oluwole, O. O. and M. L. Davila, J Leukoc Biol, 2016. 100(6): p. 1265-1272). In general, binding of the cell surface-expressed chimeric receptor to the appropriate target results in clustering and activation of the CAR-presenting cells.

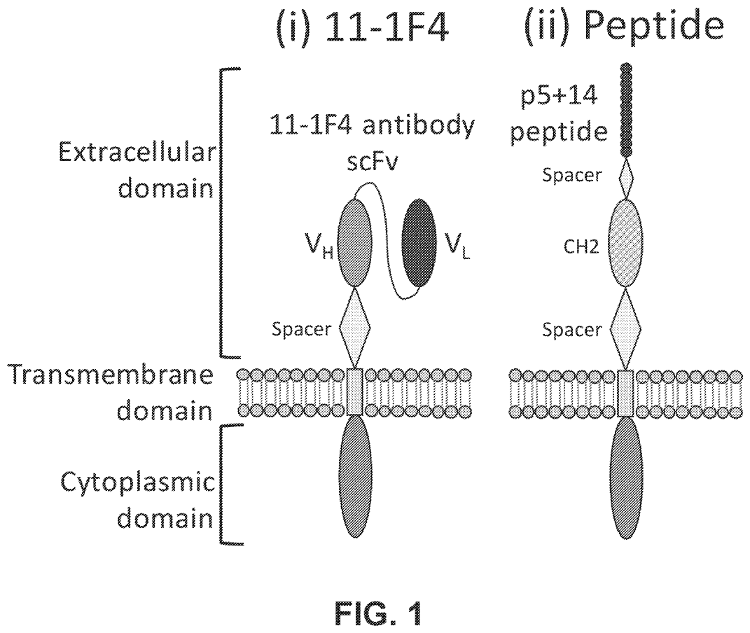

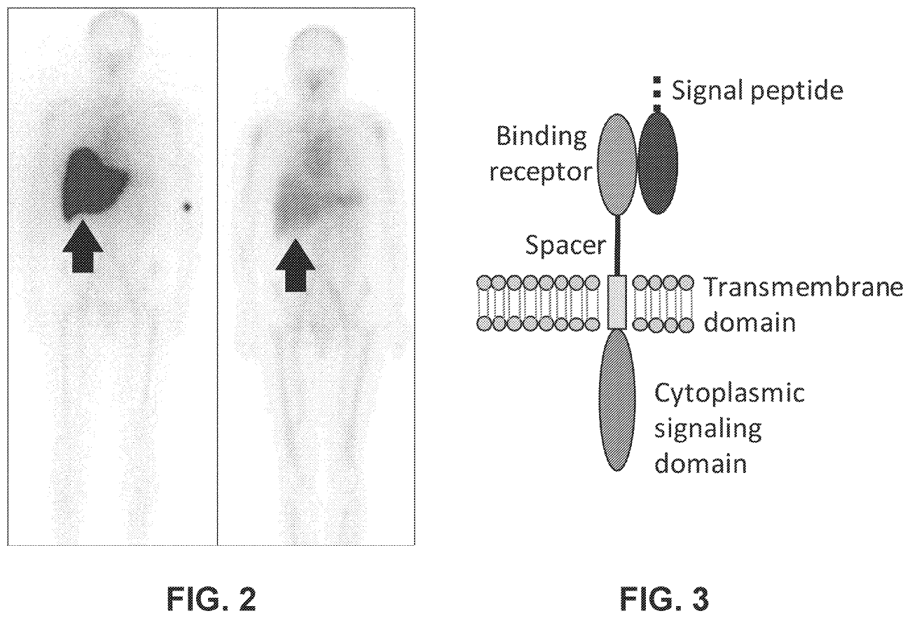

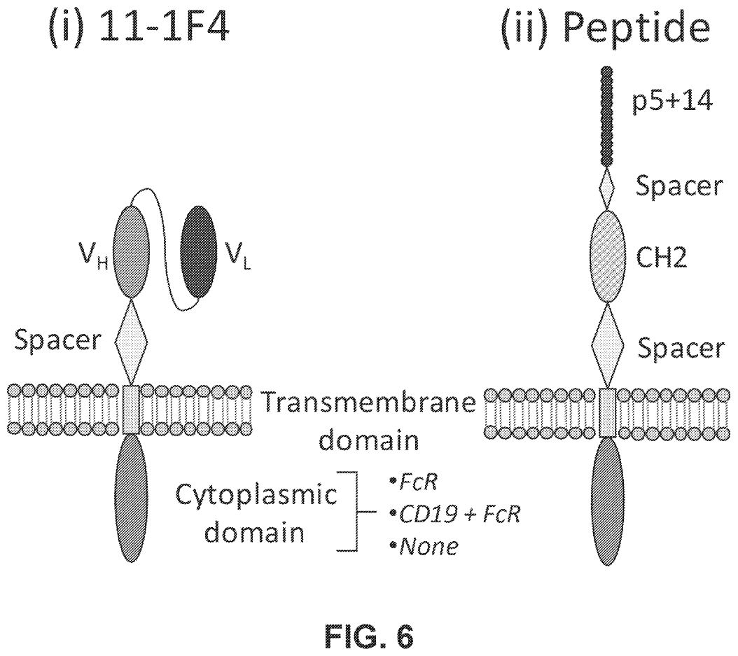

As described in detail herein, chimeric receptors (e.g., chimeric antigen receptors, or “CAR”) constructs were designed for specifically recognizing and promoting the phagocytosis of amyloid, such as AL amyloid. The constructs may be expressed in macrophages. As described in the Examples, CARs were designed incorporating either an amyloid reactive single-chain variable fragment (scFv) or an amyloid reactive synthetic peptide as the target binding receptor (see. e.g., FIG. 1 and FIG. 6; Wall, J. S., et al., Molecules, 2015. 20(5): p. 7657-82; Wall, J. S., et al., Proc Natl Acad Sci USA, 2018. 115(46): p. E10839-E10848). It is believed that amyloid is an excellent and untapped target for this approach given that it is a devastating pathology that is acellular, and therefore lacks “don't eat me” proteins associated with tumor cells (e.g. CD47 (se Gu, S., et al., J Immunol Res, 2018. 2018: p. 6156757; Russ, A., et al., Blood Rev, 2018. 32(6): p. 480-489; Tong, B. and M. Wang, Future Oncol, 2018. 14(21): p. 2179-2188) and MHC class I (see Barkal, A. A., et al., Nat Immunol, 2018. 19(1): p. 7644). Further, amyloid is readily accessible from the vasculature.

Provided herein are chimeric receptors that bind amyloid (e.g., human amyloid fibrils). In some embodiments, the chimeric receptor comprises a cytoplasmic domain, wherein the cytoplasmic domain comprises a signaling domain of a receptor that when activated activates a phagocytic cell (e.g., a macrophage); a transmembrane domain; and an extracellular domain, wherein the extracellular domain comprises an amyloid binding region.

A. Extracellular Domains Comprising Amyloid-Binding Regions

Provided herein are chimeric receptors comprising an extracellular domain. In some embodiments, the extracellular domain comprises a region interacts with or otherwise binds to a region, such as an epitope, of a human amyloid fibril. In some embodiments, the amyloid-binding regions described herein bind to amyloid deposits or fibrils (e.g., human amyloid deposits or fibrils). In some embodiments, the amyloid-binding region binds to one or more amyloidogenic peptides in amyloids. In some embodiments, amyloids bound by the amyloid-binding region comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the amyloid-binding region comprise amyloidogenic forms of immunoglobulin heavy chain (AH), β₂-microglobulin (Aβ₂M), transthyretin variants (ATTR), apolipoprotein AI(AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, Alλ1 other amyloidogenic peptides. The amyloidogenic peptides bound by the amyloid-binding region can be a protein, a protein fragment, or a protein domain. In some embodiments, the amyloid deposits or amyloid fibrils comprise recombinant amyloidogenic proteins. In some embodiments, the amyloids are part of the pathology of a disease.

1. Amyloid Binding Regions Comprising Amyloid-Binding Peptides or Functional Fragment Thereof

Provided herein are chimeric receptors comprising an extracellular domain comprising an amyloid-binding region. In some embodiments, the amyloid binding region comprises an amyloid-binding peptide or functional fragment thereof. In some embodiments, the amyloid-binding region comprises an amyloid-binding peptide or functional fragment thereof as set forth in Table A. In some embodiments, the amyloid-binding peptide or functional fragment thereof comprises the amino acid sequence of P5, P5R, P5G, P8, P9, P19, P20, P31, P37, P39, P42, P43, P44, P48, P50, P58, P5+14, or p5R+14, as shown in Table A. In some embodiments, the amyloid-binding peptide is P5, P5R, P5G, P8, P9, P19, P20, P31, P37, P39, P42, P43, P44, P48, P50, P58, P5+14, or p5R+14, as shown in Table A. Without wishing to be bound by any particular theory, it is believed that the amyloid-binding peptide or functional fragment thereof targets the chimeric receptor to the amyloid deposits.

TABLE A
Example Amyloid-Binding Peptide Sequences

| PEPTIDE | PRIMARY SEQUENCE: | SEQ ID NO: |
|---|---|---|
| PS | KAQKA QAKQA KQAQK | SEQ ID NO: 1 |
| | AQKAQ AKQAK Q | |
| PSR | RAQRA QARQA RQAQR | SEQ ID NO: 2 |
| | AQRAQ ARQAR Q | |
| P5G | GAQGA QAGQA GQAQG | SEQ ID NO: 3 |
| | AQGAQ AGQAG Q | |
| P8 | KAKAK AKAKA KAKAK | SEQ ID NO: 4 |
| P9 | KAQAK AQAKA QAKAQ | SEQ ID NO: 5 |
| | AKAQA KAQAK AQAK | |
| P19 | KAQQA QAKQA QQAQK | SEQ ID NO: 6 |
| | AQQAQ AKQAQ Q | |
| P20 | QAQKA QAQQA KQAQQ | SEQ ID NO: 7 |
| | AQKAQ AQQAK Q | |
| P31 | KAQKA QAKQA KQAQK | SEQ ID NO: 8 |
| | AQKAQ AKQAK Q | |
| P37 | KTVKT VTKVT KVTVK | SEQ ID NO: 7 |
| | TVKTV TKVTK V | |
| P39 | [KAQKA QAKQA KQAQK | SEQ ID NO: 10 |
| | AQKAQ AKQAK Q]_{D} | |
| P42 | V[Y]_{D}KVK TKVKT KVKTK | SEQ ID NO: 11 |
| | VKT | |
| P43 | [AQA]_{D}YS KAQKA QAKQA | SEQ ID NO: 12 |
| | KQAQK AQKAQ AKQAK Q | |
| P44 | [AQA]YA RAQRA QARQA | SEQ ID NO: 13 |
| | RQAQR AQRAQ ARQAR Q | |
| P48 | AQA[Y]_{D}S KAQKA QAKQA | SEQ ID NO: 14 |
| | KQAQK AQKAQ AKQAK Q]_{D} | |
| P50 | AQAYS KAQKA QAKQA | SEQ ID NO: 15 |
| | KQAQK AQKAQ AKQAK Q | |
| P58 | AQA[Y]_{D}S KAQKA QAKQA | SEQ ID NO: 16 |
| | KQAQK AQKAQ AKQAK Q | |
| P5 + 14 | KAQKA QAKQA KQAQK | SEQ ID NO: 17 |
| | AQKAQ AKQAK QAQKA | |
| | QKAQA KQAKQ | |
| p5R + 14 | RAQRA QARQA RQAQR | SEQ ID NO: 18 |
| | AQRAQ ARQAR QAQRA | |
| | QRAQA RQARQ | |

Where D = the “D form” enantiomer.

In some embodiments, the amyloid-binding peptide or functional fragment thereof of the chimeric receptors described herein include an amino acid sequence that is at least 80%, 85%, 90% or more identical to the amino acid sequence set forth as any one of SEQ ID NOS: 1-18, such as at least 95%, at least 96%, at least 97%, at least 98%, at least 99%, or 100% identical to the amino acid sequence set forth as any one of SEQ ID NOS: 1-18. In some embodiments, the amyloid-binding peptide or functional fragment thereof comprises or consists of from about 10 to about 55 amino acids. In some embodiments, the amyloid-binding peptide or functional fragment thereof comprises or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in International Publication No. WO2016032949, which is hereby incorporated herein in its entirety.

In some embodiments, the amyloid-binding peptide or functional fragment comprises the amino acid sequence of SEQ ID NO:1. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:1. In certain embodiments, the amyloid-binding peptide or functional fragment thereof comprises an amino acid sequence containing substitutions (e.g., conservative substitutions), insertions, or deletions relative to the amino acid sequence of SEQ ID NO:1, but retaining the ability to bind amyloid as an amyloid-binding peptide comprising the amino acid sequence of SEQ ID NO:1. In certain embodiments, a total of 1 to 5 amino acids have been substituted, inserted and/or deleted in SEQ ID NO:1.

In some embodiments, the amyloid-binding peptide or functional fragment thereof comprises the amino acid sequence of SEQ ID NO:2. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:2. In certain embodiments, the amyloid-binding peptide or functional fragment thereof comprises an amino acid sequence containing substitutions (e.g., conservative substitutions), insertions, or deletions relative to the amino acid sequence of SEQ ID NO:2, but retaining the ability to bind amyloid as an amyloid-binding peptide comprising the amino acid sequence of SEQ ID NO:2. In certain embodiments, a total of 1 to 5 amino acids have been substituted, inserted and/or deleted in SEQ ID NO:2.

In some embodiments, the amyloid-binding peptide or functional fragment thereof comprises the amino acid sequence of SEQ ID NO:3. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:3. In certain embodiments, the amyloid-binding peptide or functional fragment thereof comprises an amino acid sequence containing substitutions (e.g., conservative substitutions), insertions, or deletions relative to the amino acid sequence of SEQ ID NO:3, but retaining the ability to bind amyloid as an amyloid-binding peptide comprising the amino acid sequence of SEQ ID NO:3. In certain embodiments, a total of 1 to 5 amino acids have been substituted, inserted and/or deleted in SEQ ID NO:3.

In some embodiments, the amyloid-binding peptide or functional fragment thereof comprises the amino acid sequence of SEQ ID NO:4. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:4. In certain embodiments, the amyloid-binding peptide or functional fragment thereof comprises an amino acid sequence containing substitutions (e.g., conservative substitutions), insertions, or deletions relative to the amino acid sequence of SEQ ID NO:4, but retaining the ability to bind amyloid as an amyloid-binding peptide comprising the amino acid sequence of SEQ ID NO:4. In certain embodiments, a total of 1 to 5 amino acids have been substituted, inserted and/or deleted in SEQ ID NO:4.

In some embodiments, the amyloid-binding peptide or functional fragment thereof comprises the amino acid sequence of SEQ ID NO:5. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:5. In certain embodiments, the amyloid-binding peptide or functional fragment thereof comprises an amino acid sequence containing substitutions (e.g., conservative substitutions), insertions, or deletions relative to the amino acid sequence of SEQ ID NO:5, but retaining the ability to bind amyloid as an amyloid-binding peptide comprising the amino acid sequence of SEQ ID NO:5. In certain embodiments, a total of 1 to 5 amino acids have been substituted, inserted and/or deleted in SEQ ID NO:5.

In some embodiments, the amyloid-binding peptide or functional fragment thereof comprises the amino acid sequence of SEQ ID NO:6. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:6. In certain embodiments, the amyloid-binding peptide or functional fragment thereof comprises an amino acid sequence containing substitutions (e.g., conservative substitutions), insertions, or deletions relative to the amino acid sequence of SEQ ID NO:6, but retaining the ability to bind amyloid as an amyloid-binding peptide comprising the amino acid sequence of SEQ ID NO:6. In certain embodiments, a total of 1 to 5 amino acids have been substituted, inserted and/or deleted in SEQ ID NO:6.

In some embodiments, the amyloid-binding peptide or functional fragment thereof comprises the amino acid sequence of SEQ ID NO:7. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:7. In certain embodiments, the amyloid-binding peptide or functional fragment thereof comprises an amino acid sequence containing substitutions (e.g., conservative substitutions), insertions, or deletions relative to the amino acid sequence of SEQ ID NO:7, but retaining the ability to bind amyloid as an amyloid-binding peptide comprising the amino acid sequence of SEQ ID NO:7. In certain embodiments, a total of 1 to 5 amino acids have been substituted, inserted and/or deleted in SEQ ID NO:7.

In some embodiments, the amyloid-binding peptide or functional fragment thereof comprises the amino acid sequence of SEQ ID NO:8. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:8. In certain embodiments, the amyloid-binding peptide or functional fragment thereof comprises an amino acid sequence containing substitutions (e.g., conservative substitutions), insertions, or deletions relative to the amino acid sequence of SEQ ID NO:8, but retaining the ability to bind amyloid as an amyloid-binding peptide comprising the amino acid sequence of SEQ ID NO:8. In certain embodiments, a total of 1 to 5 amino acids have been substituted, inserted and/or deleted in SEQ ID NO:8.

In some embodiments, the amyloid-binding peptide or functional fragment thereof comprises the amino acid sequence of SEQ ID NO:9. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:9. In certain embodiments, the amyloid-binding peptide or functional fragment thereof comprises an amino acid sequence containing substitutions (e.g., conservative substitutions), insertions, or deletions relative to the amino acid sequence of SEQ ID NO:9, but retaining the ability to bind amyloid as an amyloid-binding peptide comprising the amino acid sequence of SEQ ID NO:9. In certain embodiments, a total of 1 to 5 amino acids have been substituted, inserted and/or deleted in SEQ ID NO:9.

In some embodiments, the amyloid-binding peptide or functional fragment thereof comprises the amino acid sequence of SEQ ID NO:10. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:10. In certain embodiments, the amyloid-binding peptide or functional fragment thereof comprises an amino acid sequence containing substitutions (e.g., conservative substitutions), insertions, or deletions relative to the amino acid sequence of SEQ ID NO:10, but retaining the ability to bind amyloid as an amyloid-binding peptide comprising the amino acid sequence of SEQ ID NO:10. In certain embodiments, a total of 1 to 5 amino acids have been substituted, inserted and/or deleted in SEQ ID NO:10.

In some embodiments, the amyloid-binding peptide or functional fragment thereof comprises the amino acid sequence of SEQ ID NO:11. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:11. In certain embodiments, the amyloid-binding peptide or functional fragment thereof comprises an amino acid sequence containing substitutions (e.g., conservative substitutions), insertions, or deletions relative to the amino acid sequence of SEQ ID NO: 11, but retaining the ability to bind amyloid as an amyloid-binding peptide comprising the amino acid sequence of SEQ ID NO: 11. In certain embodiments, a total of 1 to 5 amino acids have been substituted, inserted and/or deleted in SEQ ID NO:11.

In some embodiments, the amyloid-binding peptide or functional fragment thereof comprises the amino acid sequence of SEQ ID NO:12. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:12. In certain embodiments, the amyloid-binding peptide or functional fragment thereof comprises an amino acid sequence containing substitutions (e.g., conservative substitutions), insertions, or deletions relative to the amino acid sequence of SEQ ID NO:12, but retaining the ability to bind amyloid as an amyloid-binding peptide comprising the amino acid sequence of SEQ ID NO:12. In certain embodiments, a total of 1 to 5 amino acids have been substituted, inserted and/or deleted in SEQ ID NO:12.

In some embodiments, the amyloid-binding peptide or functional fragment thereof comprises the amino acid sequence of SEQ ID NO:13. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:13. In certain embodiments, the amyloid-binding peptide or functional fragment thereof comprises an amino acid sequence containing substitutions (e.g., conservative substitutions), insertions, or deletions relative to the amino acid sequence of SEQ ID NO:13, but retaining the ability to bind amyloid as an amyloid-binding peptide comprising the amino acid sequence of SEQ ID NO:13. In certain embodiments, a total of 1 to 5 amino acids have been substituted, inserted and/or deleted in SEQ ID NO:13.

In some embodiments, the amyloid-binding peptide or functional fragment thereof comprises the amino acid sequence of SEQ ID NO:14. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:14. In certain embodiments, the amyloid-binding peptide or functional fragment thereof comprises an amino acid sequence containing substitutions (e.g., conservative substitutions), insertions, or deletions relative to the amino acid sequence of SEQ ID NO:14, but retaining the ability to bind amyloid as an amyloid-binding peptide comprising the amino acid sequence of SEQ ID NO:14. In certain embodiments, a total of 1 to 5 amino acids have been substituted, inserted and/or deleted in SEQ ID NO:14.

In some embodiments, the amyloid-binding peptide or functional fragment thereof comprises the amino acid sequence of SEQ ID NO:15. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:15. In certain embodiments, the amyloid-binding peptide or functional fragment thereof comprises an amino acid sequence containing substitutions (e.g., conservative substitutions), insertions, or deletions relative to the amino acid sequence of SEQ ID NO:15, but retaining the ability to bind amyloid as an amyloid-binding peptide comprising the amino acid sequence of SEQ ID NO:15. In certain embodiments, a total of 1 to 5 amino acids have been substituted, inserted and/or deleted in SEQ ID NO:15.

In some embodiments, the amyloid-binding peptide or functional fragment thereof comprises the amino acid sequence of SEQ ID NO:16. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:16. In certain embodiments, the amyloid-binding peptide or functional fragment thereof comprises an amino acid sequence containing substitutions (e.g., conservative substitutions), insertions, or deletions relative to the amino acid sequence of SEQ ID NO:16, but retaining the ability to bind amyloid as an amyloid-binding peptide comprising the amino acid sequence of SEQ ID NO:16. In certain embodiments, a total of 1 to 5 amino acids have been substituted, inserted and/or deleted in SEQ ID NO:16.

In some embodiments, the amyloid-binding peptide or functional fragment thereof comprises the amino acid sequence of SEQ ID NO:17. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:17. In certain embodiments, the amyloid-binding peptide or functional fragment thereof comprises an amino acid sequence containing substitutions (e.g., conservative substitutions), insertions, or deletions relative to the amino acid sequence of SEQ ID NO:17, but retaining the ability to bind amyloid as an amyloid-binding peptide comprising the amino acid sequence of SEQ ID NO:17. In certain embodiments, a total of 1 to 5 amino acids have been substituted, inserted and/or deleted in SEQ ID NO:17.

In some embodiments, the amyloid-binding peptide or functional fragment thereof comprises the amino acid sequence of SEQ ID NO:18. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:18. In certain embodiments, the amyloid-binding peptide or functional fragment thereof comprises an amino acid sequence containing substitutions (e.g., conservative substitutions), insertions, or deletions relative to the amino acid sequence of SEQ ID NO:18, but retaining the ability to bind amyloid as an amyloid-binding peptide comprising the amino acid sequence of SEQ ID NO:18. In certain embodiments, a total of 1 to 5 amino acids have been substituted, inserted and/or deleted in SEQ ID NO:18.

In some embodiments, the extracellular domain comprises multiple amyloid binding peptides. In some embodiments, the amyloid binding peptides are organized in an array (i.e. one after the other).

The amino acids forming all or a part of the amyloid-binding peptide or functional fragment thereof may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids.

In some embodiments, the extracellular domain comprises a globular protein domain. In some embodiments, the globular protein domain acts as a spacer to position the amyloid-binding peptide or functional fragment thereof away from the transmembrane domain of the receptor, and therefore away from the surface of a cell comprising the chimeric receptor. In some embodiments, the globular protein domain is about 100, 101, 102, 103, 104, 105, 106, 107, 108, 109, 110, 111, 112, 113, 114, or 115 amino acids in length. In some embodiments, the globular protein domain is an immunoglobulin domain. In some embodiments, the globular protein domain is inert. In some embodiments, the globular protein domain lacks specific binding for a substrate. In some embodiments, the globular protein domain is a heavy chain constant domain or a fragment thereof, such as a CH2 domain or a fragment thereof. In some embodiments, the globular protein domain is a fluorescent protein, e.g., GFP. In some embodiments, the globular protein domain is a carrier proteins.

In some embodiments, the extracellular domain further comprises an immunoglobulin constant domain or a fragment thereof. In some embodiments, the extracellular domain comprises a heavy chain constant domain or a fragment thereof: In some embodiments, the extracellular domain comprises a CH2 domain or a fragment thereof. In some embodiments, the CH2 domain or fragment thereof is a mouse CH2 domain or a fragment thereof. In some embodiments, the CH2 domain or fragment thereof is a human CH2 domain or a fragment thereof. In some embodiments, the CH2 domain or fragment thereof is an IgG2 CH2 domain or a fragment thereof.

In some embodiments, the amyloid binding peptide or functional fragment thereof is joined directly or indirectly to an immunoglobulin constant domain or fragment thereof. In some embodiments, the amyloid binding peptide or functional fragment thereof is joined directly or indirectly to a heavy chain constant domain or fragment thereof. In some embodiments, the amyloid binding peptide or functional fragment thereof is joined directly or indirectly to a CH2 domain or fragment thereof. In some embodiments, the CH2 domain or fragment thereof is a mouse CH2 domain. In some embodiments, the CH2 domain or fragment thereof is a human CH2 domain. In some embodiments, the CH2 domain or fragment thereof is an IgG2 CH2 domain. In some embodiments, the CH2 domain or fragment thereof is derived from the pFuse vector.

In some embodiments, the CH2 domain or fragment thereof comprises an amino acid sequence having at least 80, 85, 90, 95, 97, 98, or 99% sequence identity to the CH2 domain sequence set forth in Table 2 or Table 3.

In some embodiments, the CH2 domain or fragment thereof comprises the amino acid set of SEQ ID NO:33. In some embodiments, the CH2 domain or fragment thereof comprises an amino acid sequence having at least 80, 85, 90, 95, 97, 98, or 99% sequence identity to the amino acid set forth in SEQ ID NO:33. In some embodiments, the CH2 domain or fragment thereof comprises an amino acid sequence having at least 90%, 91%, 92%, 93%, 94%, 95%, 96%, 97%, 98%, 99%, or 100% sequence identity to the amino acid sequence of SEQ ID NO:33. In certain embodiments, the CH2 domain or fragment thereof comprises an amino acid sequence containing substitutions (e.g., conservative substitutions), insertions, or deletions relative to the amino acid sequence of SEQ ID NO:33. In certain embodiments, a total of 1 to 10 amino acids have been substituted, inserted and/or deleted in SEQ ID NO:33.

In some embodiments, the extracellular domain comprises an amyloid-binding region joined to a spacer. In some embodiments, the spacer is N- and/or C-terminal of the amyloid binding region. In some embodiments the spacer comprises or consists of from about 3 to about 55 amino acids. The spacer peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker. In some embodiments, the spacer comprises the amino acid sequence of VTPTV (SEQ ID NO:36). In some embodiments, the amyloid-binding region joined to a spacer comprises the amino acid sequence of SEQ ID NO:32. In some embodiments, the amyloid-binding region joined to a spacer comprises the amino acid sequence of SEQ ID NO:39.

In some embodiments, the extracellular domain comprises an N-terminal secretory leader sequence. In some embodiments, the N-terminal secretory leader sequence comprises a fragment of CD8. In some embodiments, the N-terminal secretory leader sequence comprises a fragment of the CD8 hinge domain. In some embodiments, the N-terminal secretory leader sequence comprises the amino acid sequence of SEQ ID NO:38. In some embodiments, the N-terminal secretory leader comprises an amino acid sequence having at least 80, 85, 90, 95, 97, 98, or 99% sequence identity to the amino acid set forth in SEQ ID NO:38.

In some embodiments, the extracellular domain comprises an amyloid-binding region comprising, from N- to C-terminus, an amyloid binding peptide or functional fragment thereof, and a constant domain. In some embodiments, the extracellular domain comprises an amyloid-binding region comprising, from N- to C-terminus, an amyloid binding peptide or functional fragment thereof, a spacer, and a constant domain. In some embodiments, the extracellular domain comprises an amyloid-binding region comprising, from N- to C-terminus, an N-terminal secretory leader sequence, an amyloid binding peptide or functional fragment thereof, a spacer, and a constant domain. In some embodiments, the extracellular domain comprises an amyloid-binding region comprising, from N- to C-terminus, an amyloid binding peptide or functional fragment thereof, and a CH2 domain. In some embodiments, the extracellular domain comprises an amyloid-binding region comprising, from N- to C-terminus, an amyloid binding peptide or functional fragment thereof, a spacer, and a CH2 domain. In some embodiments, the extracellular domain comprises an amyloid-binding region comprising, from N- to C-terminus, an N-terminal secretory leader sequence an amyloid binding peptide or functional fragment thereof, a spacer, and a CH2 domain. In some embodiments, the extracellular domain comprises an amyloid-binding region comprising, from N- to C-terminus, an N-terminal secretory leader sequence, an amyloid binding peptide or functional fragment thereof, a spacer, a CH2 domain and a second spacer.

In some embodiments, the extracellular domain comprises an amyloid-binding region as shown in Table 2 (e.g., comprising the amino acid sequences of the p5+14-spacer and CH2 domain as shown in Table 2).

In some embodiments, the extracellular domain comprises an amyloid-binding region comprising the amino acid sequence of SEQ ID NO:37. In some embodiments, the amyloid-binding region comprises an amino acid sequence having at least 80, 85, 90, 95, 97, 98, or 99% sequence identity to the amino acid set forth in SEQ ID NO:37. In some embodiments, the amyloid-binding region comprises an amino acid sequence having at least 90%, 91%, 92%, 93%, 94%, 95%, 96%, 97%, 98%, 99%, or 100% sequence identity to the amino acid sequence of SEQ ID NO:37. In certain embodiments, the amyloid-binding region comprises an amino acid sequence containing substitutions (e.g., conservative substitutions), insertions, or deletions relative to the amino acid sequence of SEQ ID NO:37, but retaining the ability to bind amyloid as an amyloid-binding region comprising the amino acid sequence of SEQ ID NO:37. In certain embodiments, a total of 1 to 15 amino acids have been substituted, inserted and/or deleted in SEQ ID NO:37.

In some embodiments, the extracellular domain comprises an amyloid-binding region as shown in Table 3(e.g., comprising the extracellular domain of the “Final CAR-P Construct” as shown in Table 3).

In some embodiments, the extracellular domain comprises an amyloid-binding region comprising the amino acid sequence of SEQ ID NO:44. In some embodiments, the amyloid-binding region comprises an amino acid sequence having at least 80, 85, 90, 95, 97, 98, or 99% sequence identity to the amino acid set forth in SEQ ID NO:44. In some embodiments, the amyloid-binding region comprises an amino acid sequence having at least 90%, 91%, 92%, 93%, 94%, 95%, 96%, 97%, 98%, 99%, or 100% sequence identity to the amino acid sequence of SEQ ID NO:44. In certain embodiments, the amyloid-binding region comprises an amino acid sequence containing substitutions (e.g., conservative substitutions), insertions, or deletions relative to the amino acid sequence of SEQ ID NO:44, but retaining the ability to bind amyloid as an amyloid-binding region comprising the amino acid sequence of SEQ ID NO:44. In certain embodiments, a total of 1 to 15 amino acids have been substituted, inserted and/or deleted in SEQ ID NO:44.

In certain example embodiments, multiple of the same or different amyloid-binding peptides or functional fragments thereof can be joined to the extracellular domain.

2. Amyloid Binding Regions Derived from Antibodies that Bind Human Amyloid Fibrils

Provided herein are chimeric receptors comprising an extracellular domain comprising an amyloid binding region. In some embodiments, the amyloid binding region is an antibody or an antigen binding fragment thereof. In some embodiments, the amyloid binding region comprises a heavy chain variable region (VH). In some embodiments, the amyloid binding region comprises a light chain variable region (VL). In some embodiments, the amyloid binding region comprises an 11-1F4 antibody fragment. In some embodiments, the amyloid binding region is derived from 11-1F4.

In some embodiments, the amyloid binding region comprises one, two, three, four, five, or six CDRs of antibody 11-1F4, as shown in Table B. In some embodiments, the amyloid binding region comprises the VH and/or the VL of antibody 11-1F.

TABLE B
Amino acid sequences of 11-1F4 CDRs

| | | SEQ |
|---|---|---|
| | | ID |
| 11-1F4 CDR | Amino Acid Sequence | NO |
| CDR-L1 | RSSQSLVHRNGNTYLH | 24 |
| CDR-L2 | KVSNRFS | 25 |
| CDR-L3 | FQTTYVPNT | 26 |
| CDR-H1 | GFSLSSYGVS | 21 |
| CDR-H2 | VIWGDGSTNYHPNLMS | 22 |
| CDR-H3 | LDY | 23 |

In a particular embodiment, the amyloid binding region comprises a VH that comprises (a) a CDR-H1 comprising the amino acid sequence of SEQ ID NO:21, (b) a CDR-H2 comprising the amino acid sequence of SEQ ID NO:22, and (c) a CDR-H3 comprising the amino acid sequence of SEQ ID NO:23.

In a particular embodiment, the amyloid binding region comprises a VL that comprises (a) a CDR-L1 comprising the amino acid sequence of SEQ ID NO:24; (b) a CDR-L2 comprising the amino acid sequence of SEQ ID NO:25; and (c) a CDR-L3 comprising the amino acid sequence of SEQ ID NO:26.

In one embodiment, the amyloid binding region comprises a VL comprising the amino acid sequence of SEQ ID NO:19, and a VH comprising the amino acid sequence of SEQ ID NO:20.

In one embodiment, the amyloid binding region comprises a VL comprising the amino acid sequence of SEQ ID NO:34, and a VH comprising the amino acid sequence of SEQ ID NO:35.

In another aspect, the amyloid binding region comprises a VH comprising a CDR-H1 comprising the amino acid sequence of SEQ ID NO:21, a CDR-H2 comprising the amino acid sequence of SEQ ID NO:22, and a CDR-H3 comprising the amino acid sequence of SEQ ID NO:23; and a VL comprising a CDR-L1 comprising the amino acid sequence of SEQ ID NO:24, a CDR-L2 comprising the amino acid sequence of SEQ ID NO:25, and a CDR-L3 comprising the amino acid sequence of SEQ ID NO:26.

In another aspect, the amyloid binding region comprises a VH CDR1, a VH CDR2, and a VH CDR3 of a VH having the sequence set forth in SEQ ID NO:20 and a VL CDR1, a VL CDR2, and a VL of a VL having the sequence set forth in SEQ ID NO:19.

In another aspect, the amyloid binding region comprises a VH CDR1, a VH CDR2, and a VH CDR3 of a VH having the sequence set forth in SEQ ID NO:35 and a VL CDR1, a VL CDR2, and a VL of a VL having the sequence set forth in SEQ ID NO:35.

In some embodiments, the amyloid binding region comprises a VH sequence having at least 90%, 91%, 92%, 93%, 94%, 95%, 96%, 97%, 98%, 99%, or 100% sequence identity to the amino acid sequence of SEQ ID NO:20. In certain embodiments, the amyloid binding region comprises a VH sequence containing substitutions (e.g., conservative substitutions), insertions, or deletions relative to the amino acid sequence of SEQ ID NO:20, but retaining the ability to bind amyloid as an amyloid binding region comprising a VH comprising the amino acid sequence of SEQ ID NO:20. In certain embodiments, a total of 1 to 13 amino acids have been substituted, inserted and/or deleted in SEQ ID NO:20. In certain embodiments, substitutions, insertions, or deletions occur in regions outside the CDRs (i.e., in the FRs). In a particular embodiment, the amyloid binding region comprises a VH comprising one, two or three CDRs selected from the group consisting of: (a) a CDR-H1 comprising the amino acid sequence of SEQ ID NO:21, (b) a CDR-H2 comprising the amino acid sequence of SEQ ID NO:22, and (c) a CDR-H3 comprising the amino acid sequence of SEQ ID NO:23.

In another aspect, the amyloid binding region comprises a VL having at least 90%, 91%, 92%, 93%, 94%, 95%, 96%, 97%, 98%, 99%, or 100% sequence identity to the amino acid sequence of SEQ ID NO:19. In certain embodiments, the amyloid binding region comprises a VL sequence containing substitutions (e.g., conservative substitutions), insertions, or deletions relative to the amino acid sequence of SEQ ID NO:19, but retaining the ability to bind amyloid as an amyloid binding region comprising a VL comprising the amino acid sequence of SEQ ID NO:19. In certain embodiments, a total of 1 to 11 amino acids have been substituted, inserted and/or deleted in SEQ ID NO:19. In certain embodiments, the substitutions, insertions, or deletions occur in regions outside the CDRs (i.e., in the FRs). In a particular embodiment, the amyloid binding region comprises a VL comprising one, two or three CDRs selected from the group consisting of (a) a CDR-L1 comprising the amino acid sequence of SEQ ID NO:24; (b) a CDR-L2 comprising the amino acid sequence of SEQ ID NO:25; and (c) a CDR-L3 comprising the amino acid sequence of SEQ ID NO:26.

In some embodiments, the amyloid binding region comprises a VH sequence having at least 90%, 91%, 92%, 93%, 94%, 95%, 96%, 97%, 98%, 99%, or 100% sequence identity to the amino acid sequence of SEQ ID NO:35. In certain embodiments, the amyloid binding region comprises a VH sequence containing substitutions (e.g., conservative substitutions), insertions, or deletions relative to the amino acid sequence of SEQ ID NO:35, but retaining the ability to bind amyloid as an amyloid binding region comprising a VH comprising the amino acid sequence of SEQ ID NO:35. In certain embodiments, a total of 1 to 13 amino acids have been substituted, inserted and/or deleted in SEQ ID NO:35. In certain embodiments, substitutions, insertions, or deletions occur in regions outside the CDRs (i.e., in the FRs). In a particular embodiment, the amyloid binding region comprises a VH comprising one, two or three CDRs selected from the group consisting of: (a) a CDR-H1 comprising the amino acid sequence of SEQ ID NO:21, (b) a CDR-H2 comprising the amino acid sequence of SEQ ID NO:22, and (c) a CDR-H3 comprising the amino acid sequence of SEQ ID NO:23.

In another aspect, the amyloid binding region comprises a VL having at least 90%, 91%, 92%, 93%, 94%, 95%, 96%, 97%, 98%, 99%, or 100% sequence identity to the amino acid sequence of SEQ ID NO:34. In certain embodiments, the amyloid binding region comprises a VL sequence containing substitutions (e.g., conservative substitutions), insertions, or deletions relative to the amino acid sequence of SEQ ID NO:34, but retaining the ability to bind amyloid as an amyloid binding region comprising a VL comprising the amino acid sequence of SEQ ID NO:34. In certain embodiments, a total of 1 to 11 amino acids have been substituted, inserted and/or deleted in SEQ ID NO:34. In certain embodiments, the substitutions, insertions, or deletions occur in regions outside the CDRs (i.e., in the FRs). In a particular embodiment, the amyloid binding region comprises a VL comprising one, two or three CDRs selected from the group consisting of (a) a CDR-L1 comprising the amino acid sequence of SEQ ID NO:24; (b) a CDR-L2 comprising the amino acid sequence of SEQ ID NO:25; and (c) a CDR-L3 comprising the amino acid sequence of SEQ ID NO:26.

In some embodiments, the amyloid binding region comprises one, two, three, four, five, or six CDRs of antibody 11-1F4 with one or more conservative amino acid substitutions. In some embodiments, the amyloid binding region comprises the VH and/or the VL of antibody 11-1F4 with one or more conservative amino acid substitutions.

In some embodiments, the amyloid binding region comprises a light chain variable region (VL) and a heavy chain variable region (VH), wherein the VL comprises a CDR-L1 comprising the amino acid sequence set forth in SEQ ID NO:24 with one or more conservative amino acid substitutions, a CDR-L2 comprising the amino acid sequence set forth in SEQ ID NO:25 with one or more conservative amino acid substitutions, and a CDR-L3 comprising the amino acid sequence set forth in SEQ ID NO:26 with one or more conservative amino acid substitutions, and the VH comprises a CDR-H1 comprising the amino acid sequence set forth in SEQ ID NO:21 with one or more conservative amino acid substitutions, a CDR-H2 comprising the amino acid sequence set forth in SEQ ID NO:22 with one or more conservative amino acid substitutions, and a CDR-H3 comprising the amino acid sequence set forth in SEQ ID NO:23 with one or more conservative amino acid substitutions. In some embodiments, the amyloid binding region comprises a CDR-H1, a CDR-H2, and a CDR-H3, respectively comprising the amino acid sequences of a CDR-H1, a CDR-H2, and a CDR-H3 of a VH having the sequence set forth in SEQ ID NO:20 with one or more conservative amino acid substitutions, and a CDR-L1, a CDR-L2, and a CDR-L3, respectively comprising the amino acid sequences of a CDR-L1, a CDR-L2, and a CDR-L3 of a VL having the sequence set forth in SEQ ID NO:19 with one or more conservative amino acid substitutions.

In some embodiments, the amyloid binding region comprises a humanized antibody fragment (e.g., a humanized fragment of 11-1F4). In some embodiments, the amyloid binding region comprises a humanized scFv derived from 11-1F4

In some embodiments, the amyloid binding region comprises a VH and a VL fused by a linker. In some embodiments, the linker is a scFv linker. In some embodiments the linker comprises or consists of from about 3 to about 55 amino acids. The linker peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the linker is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the linker is a flexible linker. In some embodiments, the linker is uncharged. In some embodiments, the linker is a glycine serine linker. In some embodiments, the linker comprises the amino acid sequence of SEQ ID NO:27. In some embodiments, the amyloid binding region comprises, from N- to C-terminus, a VL, a linker, and a VH. In some embodiments, the amyloid binding region comprises, from N- to C-terminus, a VH, a linker, and a VL.

In some embodiments, the extracellular domain comprises an amyloid-binding region joined to a spacer. In some embodiments, the spacer is N- and/or C-terminal of the amyloid binding region. In some embodiments the spacer comprises or consists of from about 3 to about 55 amino acids. The spacer peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker. In some embodiments, the spacer comprises the amino acid sequence of SEQ ID NO:56.

In some embodiments, the amyloid binding region comprises an N-terminal secretory leader sequence. In some embodiments, the N-terminal secretory leader sequence is a cleavable N-terminal secretory leader sequence. In some embodiments, the N-terminal secretory leader sequence comprises a fragment of a CD8 α chain, e.g., a mouse CD8 α chain. In some embodiments, the N-terminal secretory leader sequence comprises residues 1-27 of mouse CD8 α chain (e.g., residues 1-27 of UniProtKB No. P01731 [CD8A_MOUSE]). In some embodiments, the N-terminal secretory leader sequence comprises the amino acid sequence of SEQ ID NO:28. In some embodiments, the N-terminal secretory leader sequence comprises an amino acid sequence having at least 80, 85, 90, 95, 97, 98, or 99% sequence identity to the amino acid set forth in SEQ ID NO:28. In some embodiments, the extracellular domain comprises, from N- to C-terminus, an N-terminal secretory leader sequence, a VL, a linker, and a VH. In some embodiments, the extracellular domain comprises, from N- to C-terminus, an N-terminal secretory leader sequence, a VH, a linker, and a VL.

In some embodiments, the amyloid binding region is a single-chain variable fragment (scFv). In some embodiments, the scFv comprises a VH and a VL. In some embodiments, the VH and/or the VL are any one of the VHs and VLs described herein. In some embodiments, the scFv comprises a VL comprising the amino acid sequence of SEQ ID NO:19, and a VH comprising the amino acid sequence of SEQ ID NO:20. In some embodiments, the scFv comprises a VL comprising the amino acid sequence of SEQ ID NO:35, and a VH comprising the amino acid sequence of SEQ ID NO:35. In some embodiments, the scFv comprises, from N- to C-terminus, a VL, a linker, and a VH. In some embodiments, the linker comprises the amino acid sequence of SEQ ID NO:27. In some embodiments, the scFv comprises, from N- to C-terminus, an N-terminal secretory leader sequence, a VL, a linker, and a VH. In some embodiments, the N-terminal secretory leader sequence comprises the amino acid sequence of SEQ ID NO:28. In some embodiments, the N-terminal secretory leader sequence comprises an amino acid sequence having at least 80, 85, 90, 95, 97, 98, or 99% sequence identity to the amino acid set forth in SEQ ID NO:28.

In some embodiments, the extracellular domain comprises an amyloid-binding region comprising a scFv. In some embodiments, the scFv comprises the amino acid sequence of SEQ ID NO:47. In some embodiments, the scFv comprises an amino acid sequence having at least 80, 85, 90, 95, 97, 98, or 99% sequence identity to the amino acid set forth in SEQ ID NO:47. In some embodiments, the scFv comprises an amino acid sequence having at least 90%, 91%, 92%, 93%, 94%, 95%, 96%, 97%, 98%, 99%, or 100% sequence identity to the amino acid sequence of SEQ ID NO:47. In certain embodiments, the scFv comprises an amino acid sequence containing substitutions (e.g., conservative substitutions), insertions, or deletions relative to the amino acid sequence of SEQ ID NO:47, but retaining the ability to bind amyloid as an amyloid-binding region comprising the amino acid sequence of SEQ ID NO:47. In certain embodiments, a total of 1 to 15 amino acids have been substituted, inserted and/or deleted in SEQ ID NO:47.

In some embodiments, the extracellular domain comprises an amyloid-binding region comprising a scFv. In some embodiments, the scFv comprises the amino acid sequence of SEQ ID NO:48. In some embodiments, the scFv comprises an amino acid sequence having at least 80, 85, 90, 95, 97, 98, or 99% sequence identity to the amino acid set forth in SEQ ID NO:48. In some embodiments, the scFv comprises an amino acid sequence having at least 90%, 91%, 92%, 93%, 94%, 95%, 96%, 97%, 98%, 99%, or 100% sequence identity to the amino acid sequence of SEQ ID NO:48. In certain embodiments, the scFv comprises an amino acid sequence containing substitutions (e.g., conservative substitutions), insertions, or deletions relative to the amino acid sequence of SEQ ID NO:48, but retaining the ability to bind amyloid as an amyloid-binding region comprising the amino acid sequence of SEQ ID NO:48. In certain embodiments, a total of 1 to 15 amino acids have been substituted, inserted and/or deleted in SEQ ID NO:48.

In some embodiments, the extracellular domain comprises, from N- to C-terminus, a VL, a linker, a VH, and a spacer. In some embodiments, the extracellular domain comprises, from N- to C-terminus, a VH, a linker, a VL, and a spacer. In some embodiments, the extracellular domain comprises, from N- to C-terminus, a scFv and a spacer. An exemplary structure of an extracellular domain is diagrammed in FIG. 6 (see, e.g., diagram (i) 11-1F4).

In some embodiments, the amyloid binding region binds to human amyloid fibrils with a dissociation constant (K_{d}) that is less than about 100, 10, 1, 0.1, 0.01 μM. In some embodiments, the amyloid binding region binds to human amyloid fibrils with a dissociation constant (K_{d}) that is about 0.01, 0.05, 0.1, 0.2, 0.3, 0.4, 0.5. 0.6, 0.7, 0.8, 0.9, 1, 2, 3, 4, 5, 6, 7, 8, 9, 10, 15, 20, 25, 50, 75, or 100 μM including any value or range between these values. In some embodiments, the amyloid binding region binds to human amyloid fibrils with a dissociation constant (K_{d}) that is less than 500, 100, 10, or 1 nM. In some embodiments, the amyloid binding region binds to human amyloid fibrils with a dissociation constant (K_{d}) that is less than about 5, 10, 20, 30, 40, 50, 60, 70, 80, 90, 100, 250, 500, 750, 1000, 2000, or 2200 nM. In some embodiments, the amyloid binding region binds to human amyloid fibrils with a dissociation constant (K_{d}) that is about 5, 10, 20, 30, 40, 50, 60, 70, 80, 90, 100, 250, 500, 750, 1000, 2000, or 2200 nM, including any value or range between these values. In some embodiments, the amyloid binding region binds to human amyloid fibrils with a dissociation constant (K_{d}) that is about 40-50 nM. In some embodiments, the amyloid binding region binds to human amyloid fibrils with a dissociation constant (K_{d}) that is 40-50 nM. In some embodiments, the amyloid binding region binds to human amyloid fibrils with a dissociation constant (K_{d}) that is less than 50 nM. In some embodiments, the amyloid binding region binds to human amyloid fibrils with a dissociation constant (K_{d}) that is less than the K_{d} of c11-1F4 binding to human amyloid fibrils.

In some embodiments, the amyloid binding region binds to human amyloid fibrils with half-maximal binding at a concentration of antibody (EC₅₀) that is less than about 0.01, 0.1, or 1 μM. In some embodiments, the amyloid binding region binds to human amyloid fibrils with half-maximal binding at a concentration of antibody (EC₅₀) that is about 0.01, 0.05, 0.1, 0.2, 0.3, 0.4, 0.5, 0.6, 0.7, 0.8, 0.9, 1, 2, 3, 4, 5, 6, 7, 8, 9, or 10 μM, including any value or range between these values. In some embodiments, the amyloid binding region binds to human amyloid fibrils with half-maximal binding at a concentration of antibody (EC₅₀) that is less than about 1, 10, 100, or 1000 nM. In some embodiments, the amyloid binding region binds to human amyloid fibrils with half-maximal binding at a concentration of antibody (EC₅₀) that is about 1, 5, 10, 15, 20, 25, 30, 35, 40, 45, 50, 55, 60, 65, 70, 75, 80, 85, 90, 95, 100, 100, 250, 500, 750, or 1000 nM, including any value or range between these values. In some embodiments, the amyloid binding region binds to human amyloid fibrils with half-maximal binding at a concentration of antibody (EC₅₀) that is about 17 nM, 7 nM, 16 nM, 75 nM, or 95 nM. In some embodiments, the amyloid binding region binds to human amyloid fibrils with half-maximal binding at a concentration of antibody (EC₅₀) that is less than about 10 nM, 20 nM, 80 nM, or 100 nM. In some embodiments, the amyloid binding region binds to human amyloid fibrils with half-maximal binding at a concentration of antibody (EC₅₀) that is less than the EC₅₀ of c11-1F4 binding to human amyloid fibrils.

Methods for calculating dissociation constants and EC₅₀s are known in the art, and include, for example, surface plasmon resonance and EuLISAs. In some embodiments, the dissociation constant is determined by measuring binding to a Len(1-22) monomer peptide, for example, using surface plasmon resonance. In some embodiments, the EC₅₀ is determined using a EuLISA. In some embodiments, the EC₅₀ is determined using a EuLISA to measure the level of binding to rVλ6Wil fibrils, Perl25 wtATTR extract, Ken ATTR extract. SHI ALλ liver extract, or TAL ALκ liver extract.

B. Transmembrane Domains

Provided herein are chimeric receptors comprising a transmembrane domain. In some embodiments, the transmembrane domain connects the extracellular domain to the cytoplasmic domain.

The transmembrane domain may be derived either from a naturally occurring protein or from a synthetic source. In some embodiments in which the source is a naturally occurring protein, the transmembrane domain may be derived from any membrane-bound or transmembrane protein. In some embodiments, the transmembrane domain is derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some embodiments, the transmembrane domain comprises a hinge, e.g., a human Ig (immunoglobulin) hinge.

In some embodiments, the transmembrane domain is fused to an N-terminal spacer (e.g., a spacer between the extracellular domain and the transmembrane domain, as diagrammed in FIG. 6). In some embodiments the spacer comprises or consists of from about 3 to about 55 amino acids. The spacer peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker. In some embodiments, the spacer comprises the amino acid sequence of SEQ ID NO:46.

In some embodiments, the transmembrane domain comprises an amino acid sequence as shown in Table 1. In some embodiments, the transmembrane domain comprises an amino acid sequence as shown in Table 3. In some embodiments, the transmembrane domain is derived from CD8. In some embodiments, the transmembrane domain is derived from a CD8 α chain. In some embodiments, the transmembrane domain is derived from a mouse CD8 α chain. In some embodiments, the transmembrane domain comprises residues 148-218 from the mouse CD8 α chain (e.g., residues 148-218 of UniProtKB No. P01731). In some embodiments, the transmembrane domain comprises the amino acid sequence of SEQ ID NO:29. In some embodiments, the transmembrane domain comprises an amino acid sequence having at least 90%, 91%, 92%, 93%, 94%, 95%, 96%, 97%, 98%, 99%, or 100% sequence identity to the amino acid sequence of SEQ ID NO:29. In some embodiments, the transmembrane domain comprises the amino acid sequence of SEQ ID NO:40. In some embodiments, the transmembrane domain comprises an amino acid sequence having at least 90%, 91%, 92%, 93%, 94%, 95%, 96%, 97%, 98%, 99%, or 100% sequence identity to the amino acid sequence of SEQ ID NO:40. In some embodiments, the transmembrane domain is derived from a human CD8 α chain. In some embodiments, the transmembrane domain comprises the amino acid sequence of SEQ ID NO:57. In some embodiments, the transmembrane domain comprises an amino acid sequence having at least 90%, 91%, 92%, 93%, 94%, 95%, 96%, 97%, 98%, 99%, or 100% sequence identity to the amino acid sequence of SEQ ID NO:57.

In some embodiments, the transmembrane domain is a synthetic transmembrane domain. In some embodiments, in which the transmembrane domain is synthetic, the transmembrane domain comprises predominantly hydrophobic residues such as leucine and valine. In some embodiments, the transmembrane domain comprises a triplet of phenylalanine, tryptophan and valine each end of a synthetic transmembrane domain.

C. Cytoplasmic Domains

Provided herein are chimeric receptors comprising a cytoplasmic domain. In some embodiments, the cytoplasmic domain comprises a signaling domain of a receptor that, when activated, activates a phagocytic cell (e.g., a macrophage).

In some embodiments, the cytoplasmic domain comprises a phagocytosis signaling domain. In some embodiments, the cytoplasmic domain comprises an immunoreceptor tyrosine-based activation motif (ITAM)(see. e.g., Morrissey, M. A., et al., Elife, 2018. 7). In some embodiments, the cytoplasmic domain comprises a fragment or region of CD19. In some embodiments, the cytoplasmic domain comprises a fragment or region of CD3ζ. In some embodiments, the cytoplasmic domain comprises a fragment or region of FcR, e.g., the FcR γ subunit. In some embodiments, the cytoplasmic domain is derived from CD19, CD3ζ, and/or FcR. Exemplary cytoplasmic domains are further described in Morrissey, M. A., et al., Elife, 2018. 7.

In some embodiments, the cytoplasmic domain comprises a cytoplasmic domain I, a cytoplasmic domain II, or a functional fragment thereof. In some embodiments, the cytoplasmic domain comprises an amino acid sequence having at least 80, 85, 90, 95, 97, 98, or 99 sequence identity to the sequence of the cytoplasmic domain I or cytoplasmic domain II set forth in Table 1 or Table 3.

In some embodiments, the cytoplasmic domain is derived from CD19. In some embodiments, a cytoplasmic domain derived from CD19 is referred to as a “cytoplasmic domain 1”. In some embodiments, the cytoplasmic domain is derived from mouse CD19. In some embodiments, the cytoplasmic domain is derived from human CD19. In some embodiments, the cytoplasmic domain comprises amino acid residues 500-534 of mouse CD19 (e.g., amino acid residues 500-534 of UniProtKB No. P25918). In some embodiments, the cytoplasmic domain comprises the amino acid sequence of SEQ ID NO:30. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:30. In certain embodiments, the cytoplasmic domain comprises an amino acid sequence containing substitutions (e.g., conservative substitutions), insertions, or deletions relative to the amino acid sequence of SEQ ID NO:30, but retaining the ability to activate a phagocytic cell as a cytoplasmic domain comprising the amino acid sequence of SEQ ID NO:30. In certain embodiments, a total of 1 to 5 amino acids have been substituted, inserted and/or deleted in SEQ ID NO:30. In some embodiments, the cytoplasmic domain comprises the amino acid sequence of SEQ ID NO:42. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:42. In certain embodiments, the cytoplasmic domain comprises an amino acid sequence containing substitutions (e.g., conservative substitutions), insertions, or deletions relative to the amino acid sequence of SEQ ID NO:42, but retaining the ability to activate a phagocytic cell as a cytoplasmic domain comprising the amino acid sequence of SEQ ID NO:42. In certain embodiments, a total of 1 to 5 amino acids have been substituted, inserted and/or deleted in SEQ ID NO:42.

In some embodiments, the cytoplasmic domain is derived from an Fc receptor (FcR). In some embodiments, a cytoplasmic domain derived from an Fc receptor is referred to as a “cytoplasmic domain II”. In some embodiments, the cytoplasmic domain comprises amino acid residues 19-86 of mouse Fc ERG precursor (e.g., amino acid residues 19-86 of UniProtKB No. P20491). In some embodiments, the cytoplasmic domain comprises the amino acid sequence of SEQ ID NO:31. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:31. In certain embodiments, the cytoplasmic domain comprises an amino acid sequence containing substitutions (e.g., conservative substitutions), insertions, or deletions relative to the amino acid sequence of SEQ ID NO:31, but retaining the ability to activate a phagocytic cell as a cytoplasmic domain comprising the amino acid sequence of SEQ ID NO:31. In certain embodiments, a total of 1 to 5 amino acids have been substituted, inserted and/or deleted in SEQ ID NO:31. In some embodiments, the cytoplasmic domain comprises the amino acid sequence of SEQ ID NO:41. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:41. In certain embodiments, the cytoplasmic domain comprises an amino acid sequence containing substitutions (e.g., conservative substitutions), insertions, or deletions relative to the amino acid sequence of SEQ ID NO:41, but retaining the ability to activate a phagocytic cell as a cytoplasmic domain comprising the amino acid sequence of SEQ ID NO:41. In certain embodiments, a total of 1 to 5 amino acids have been substituted, inserted and/or deleted in SEQ ID NO:41. In some embodiments, the cytoplasmic domain comprises the amino acid sequence of SEQ ID NO:45. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:45. In certain embodiments, the cytoplasmic domain comprises an amino acid sequence containing substitutions (e.g., conservative substitutions), insertions, or deletions relative to the amino acid sequence of SEQ ID NO:45, but retaining the ability to activate a phagocytic cell as a cytoplasmic domain comprising the amino acid sequence of SEQ ID NO:45. In certain embodiments, a total of 1 to 5 amino acids have been substituted, inserted and/or deleted in SEQ ID NO:45.

In some embodiments, the cytoplasmic domain is derived from CD19 and FcR. In some embodiments, the cytoplasmic domain is derived from mouse CD19 and FcR. In some embodiments, the cytoplasmic domain comprises amino acid residues 500-534 of mouse CD19 (e.g., amino acid residues 500-534 of UniProtKB No. P25918) and amino acid residues 19-86 of mouse Fc ERG precursor (e.g., amino acid residues 19-86 of UniProtKB No. P20491). In some embodiments, the cytoplasmic domain comprises the amino acid sequence of SEQ ID NO:30 and the amino acid sequence of SEQ ID NO:31. In some embodiments, the cytoplasmic domain comprises an amino acid sequence having at least 90%, 91%, 92%, 93%, 94%, 95%, 96%, 97%, 98%, 99%, or 100% sequence identity to the amino acid sequence of SEQ ID NO:30 and the amino acid sequence of SEQ ID NO:31. In certain embodiments, the cytoplasmic domain comprises an amino acid sequence containing substitutions (e.g., conservative substitutions), insertions, or deletions relative to the amino acid sequences of SEQ ID NO:30 and/or SEQ ID NO:31, but retaining the ability to activate a phagocytic cell as a cytoplasmic domain comprising the amino acid sequence of SEQ ID NO:30 and SEQ ID NO:31. In certain embodiments, a total of 1 to 5 amino acids have been substituted, inserted and/or deleted in SEQ ID NO:30 and/or SEQ ID NO:31. In some embodiments, the cytoplasmic domain comprises, from N- to C-terminus, a domain derived from FcR and a domain derived from CD19.

In some embodiments, two or more cytoplasmic domains are connected by a peptide spacer.

In some embodiments, the cytoplasmic domain is a mannose receptor, a complement receptor 1, 3 or 4, a scavenger receptor, or an FC gamma receptor.

In some embodiments, the cytoplasmic domain comprises a co-stimulatory domain. In some embodiments, the cytoplasmic domain comprises a domain derived from Toll-Like Receptor 2.

In some embodiments, binding of amyloid to the extracellular domain activates the cytoplasmic domain of the chimeric receptor. In some embodiments, activation of the cytoplasmic domain of the chimeric receptor comprises activation of the signaling domain of a receptor that. In some embodiments, activation of the cytoplasmic domain of the chimeric receptor results in the activation of a phagocytic cell (e.g., a macrophage). In some embodiments, the activated phagocytic cell phagocytoses the amyloid.

D. Full-Length Chimeric Receptor Constructs

Provided herein are chimeric receptors that bind amyloid (e.g., human amyloid fibrils). In some embodiments, the chimeric receptor comprises a cytoplasmic domain, wherein the cytoplasmic domain comprises a signaling domain of a receptor that when activated activates a phagocytic cell (e.g., a macrophage); a transmembrane domain; and an extracellular domain, wherein the extracellular domain comprises an amyloid binding region. The cytoplasmic domain, the transmembrane domain, and the extracellular domain of the chimeric receptor may be any one of the cytoplasmic domains, transmembrane domains, and extracellular domains described herein. In some embodiments, the chimeric receptor comprises, from N- to C-terminus, an extracellular domain, a transmembrane domain, and a cytoplasmic domain. In some embodiments, the chimeric receptor further comprises an N-terminal secretory leader sequence at the N-terminus of the extracellular domain. Exemplary diagrams of the structures of chimeric receptors are provided in FIG. 1 and FIG. 6, and exemplary amino acid sequences of chimeric receptors are provided in Table C.

TABLE C
Amino acid sequences of exemplary full-length chimeric receptor constructs

| | | | SEQ |
|---|---|---|---|
| Construct | | | ID |
| name | Description | Amino acid sequence | NO: |
| CAR-P | Peptide-based CAR with | MALPVTALLLPLALLLHAARPSQFRVSPTVG | 43 |
| construct- | (from N- to C-terminus) an | GGYSKAQKAQAKQAKQAQKAQKAQAKQAKQV | |
| 345aa, see | N-terminal secretory leader | TPTVPNLLGGPSVFIFPPKIKDVLMISLSPI | |
| Table 3. | sequence, p5, spacer, CH2 | VTCVVVDVSEDDPDVQISWFVNNVBVKTAQT | |
| | domain, spacer, | QTHREDYNSTLRVVSALPIQHQDWMSGKEFK | |
| | transmembrane domain, | CKVNNKDLPAPIERTISKPKTTTKPVLRTPS | |
| | cytoplasmic domain II, and | PVHPTGTSQPQRPEDCRPRGSVKGTGLDFAC | |
| | cytoplasmic domain I. | DIYIWAPLAGICVALLLSLIITLICLGEPQL | |
| | | CYILDAVLFLYGIVLTLLYCRLKIQVRKAAI | |
| | | ASREKADAVYTGLNTRSQETYETLKHEKPPQ | |
| | | LYAAPQLHSIQSGPSHEEDADSYENMDKSDD | |
| | | LEPA | |
| CAR-P | Peptide-based CAR with | MALPVTALLLPLALLLHAARPSQFRVSPTVG | 51 |
| construct | (from N-to C-terminus) an | GGYSKAQKAQAKQAKQAQKAQKAQAKQAKQV | |
| lacking | N-terminal secretory leader | TPTVPNLLGGPSVFIFPPKIKDVLMISLSPI | |
| cytoplasmic | sequence, p5, spacer, CH2 | VTCVVVDVSEDDPDVQISWFVNNVEVHTAQT | |
| domain I. | domain, spacer, | QTHREDYNSTLRVVSALPIQHQDWMSGKEFK | |
| | transmembrane domain, | CKVNNKDLPAPIERTISKPKTTTKPVLRTPS | |
| | and cytoplasmic domain II. | PVHPTGTSQPQRPEDCRPRGSVKGTGLDFAC | |
| | Lacks cytoplasmic domain | DIYIWAPLAGICVALLLSLIITLICLGEPQL | |
| | I. | CYILDAVLFLYGIVLTLLYCRLKIQVRKAAI | |
| | | ASREKADAVYTGLNTRSQETYETLKHEKPPQ | |
| CAR-P | Peptide-based CAR with | TVGGGYSKAQKAQAKQAKQAQKAQKAQAKQA | 52 |
| construct | (from N-to C-terminus) p5, | KQVTPTVPNLLGGPSVFIFPPKIKDVLMISL | |
| lacking N- | spacer, CH2 domain, | SPIVICVVVDVSEDDPDVQISWFVNNVEVHT | |
| terminal | spacer, transmembrane | AQTQTHREDYNSTLRVVSALPIQHQDWMSGK | |
| secretory leader | domain, cytoplasmic | EFKCKVNNKDLPAPIERTISKPKTTTKPVLR | |
| sequence | domain II, and cytoplasmic | TPSPVHPTGTSQPQRPEDCRPRGSVKGTGLD | |
| | domain I. | FACDIYIWAPLAGICVALLLSLIITLICLGE | |
| | Lacks N-terminal secretory | PQLCYILDAVLFLYGIVLTLLYCRLKIQVRK | |
| | leader sequence. | AAIASREKADAVYTGLNTRSQETYETLKHEK | |
| | | PPQLYAAPQLHSIQSGPSHEEDADSYENMDK | |
| | | SDDLEPA | |
| CAR-P | Peptide-based CAR with | TVGGGYSKAQKAQAKQAKQAQKAQKAQAKQA | 53 |
| construct | (from N- to C-terminus) p5, | KQVTPTVPNLLGGPSVFIFPPKIKDVLMISL | |
| lacking N- | spacer CH2 domain, spacer, | SPIVTCVVVDVSEDDPDVQISWFVNNVEVHT | |
| terminal | transmembrane domain, | AQTQTHREDYNSTLRWSALPIQHQDWMSGK | |
| secretory leader | and cytoplasmic domain II. | EFKCKVNNKDLPAPIERTISKPKTTTKPVLR | |
| sequence and | Lacks N-terminal secretory- | TPSPVHPTGTSQPQRPEDCRPRGSVKGTGLD | |
| cytoplasmic | leader sequence | FACDIYIWAPLAGIQVALLLSLIITLICLGE | |
| domain I. | cytoplasmic domain I. | PQLCYILDAVIFLYGIVLTLLYCRLKIQVRK | |
| | | AAIASREKADAVYTGLNTRSQETYETLKHEK | |
| ^{11-1F4}CAR_{tandem} | scoff-based CAR with | MASPLTRFLSLNLLLLGESIILGSGEADIVL | 50 |
| see Table 1. | (from N-to C-terminus) an | TQSPASLAVSLGQRATISYRASKSVSTSGYS | |
| | N-terminal secretory-leader | YMHWNQQKPGQPPRLLIYLVSNLESGVPARF | |
| | sequence, 11-1F4 VL, scFv | SGSGSGTDFTLNIHPVEEEDAATYYCQHIRE | |
| | linker, 11-1F4 VH, spacer, | LTRFGGGTKLEIKRGGSSRSSSSGGGGSGGG | |
| | transmembrane domain, | GQVQLKESGPGLVAPSQSLSITCTVSGFSLS | |
| | cytoplasmic domain II, and | SYGVSWVRQPPGKGLEWLGVIWGDGSTNYHP | |
| | cytoplasmic domain I. | NLMSRSLSISKDISKSQVLFKLNSLQTDDTA | |
| | | TYYCVTLDYWGQGTSVTVSKVNSTTTKPVLR | |
| | | TPSPVHPTGTSQPQRPEDCRPRGSVKGTGLD | |
| | | FACDIYIWAPLAGICVALLLSLIITLICGEP | |
| | | QLCYILDAVLFLYGIVLTLLYCRLKIQVRKA | |
| | | AIASREKADAVYTGLNTRSQETYETLKHEKP | |
| | | PQAESYENADEELAQPVGRMMDFLSPHGSAW | |
| | | DPSRE | |
| ^{11-1F4}CAR_{tandem} | scFv -based CAR with | MASPLTRFLSLNLLLLGESIILGSGEADIVL | 49 |
| lacking | (from N-to C-terminus) an | TQSPASLAVSLGQRATISYRASKSVSTSGYS | |
| cytoplasmic | N-terminal secretory leader | YMHWNQQKPGQPPRLLIYLVSNLESGVPARF | |
| domain I. | sequence, 11-1F4 VL, scFv | SGSGSGTDFTLNIHPVEEEDAATYYCQHIRE | |
| | linker, 11-1F4 VH, spacer, | LTRFGGGTKLEIKRGGSSRSSSSGGGGSGGG | |
| | transmembrane domain, | GQVQLKESGPGLVAPSQSLSITCTVSGFSLS | |
| | and cytoplasmic domain 11. | SYGVSWVRQPPGKGLEWLGVIWGDGSTNYHP | |
| | Lacks cytoplasmic domain | NLMSRSLSISKDISKSQVLFKLNSLQTDDTA | |
| | I. | TYYUVTLDYWGQGTSVTVSKVNSTTTKPVLR | |
| | | TPSPVHPTGTSQPQRPEDCRPRGSVKGTGLD | |
| | | FACDIYIWAPLAGICVALLLSLIITLICGEP | |
| | | QLCYILDAVLFLYGIVLTLLYCRLKIQVRKA | |
| | | AIASREKADAVYTGLNTRSQETYETLKHEKP | |
| ^{11-1F4}CAR_{tandem} | scFv -based CAR with | DIVLTQSPASLAVSLGQRATISYRASKSVST | 55 |
| lacking N- | (from N-to C-terminus) 11- | SGYSYMHWNQQKPGQPPRLLIYLVSNLESGV | |
| terminal | 1F4 VL, scFv linker, 11- | PARFSGSGSGTDFTLNIHPVEEEDAATYYCQ | |
| secretory leader | 1F4 VH, spacer, | HIRELTRFGGGTKLEIKRGGSSRSSSSGGGG | |
| sequence. | transmembrane domain, | SGGGGQVQLKESGPGLVAPSQSLSITCTVSG | |
| | cytoplasmic domain II, and | FSLSSYGVSWVRQPPGKGLEWLGVIWGDGST | |
| | cytoplasmic domain I. | NYHPNLMSRSLSISKDISKSQVLFKLNSLQT | |
| | Lacks N-terminal secretory | DDTATYYCVTLDYWGQGTSVTVSKVNSTTTK | |
| | leader sequence. | PVLRTPSPVHPTGTSQPQRPEDCRPRGSVKG | |
| | | TGLDFACDIYIWAPLAGICVALLLSLIITLI | |
| | | CGEPQLCYILDAVLFLYGIVLTLLYCRLKIQ | |
| | | VRKAAIASREKADAVYTGLNTRSQETYETLK | |
| | | HEKPPQAESYENADEELAQPVGRMMDFLSPH | |
| | | GSAWDPSRE | |
| ^{11-1F4}CAR_{tandem} | scFv-based CAR with | DIVLTQSPASLAVSLGQRATISYRASKSVST | 54 |
| lacking N- | (from N- to C-terminus) 11- | SGYSYMHWNQQKPGQPPRLLIYLVSNLESGV | |
| terminal | 1F4 VL, scFv linker, 11- | PARFSGSGSGTDFTLNIHPVEEEDAATYYCQ | |
| secretory leader | 1F4 VH, spacer, | HIRELTRFGGGTKLEIKRGGSSRSSSSGGGG | |
| sequence and | transmembrane domain, | SGGGGQVQLKESGPGLVAPSQSLSITCTVSG | |
| cytoplasmic | and cytoplasmic domain II. | FSLSSYGVSWVRQPPGKGLEWLGVIWGDGST | |
| domain I | Lacks N-terminal secretory' | NYHPNLMSRSLSISKDISKSQVLFKLNSLQT | |
| | leader sequence and | DDTATYYCVTLDYWGQGTSVTVSKVNSTTTK | |
| | cytoplasmic domain I. | PVLRTPSPVHPTGTSQPQRPEDCRPRGSVKG | |
| | | TGLDFACDIYIWAPLAGICVALLLSLIITLI | |
| | | CGEPQLCYILDAVLFLYGIVLTLLYCRLKIQ | |
| | | VRKAAIASREKADAVYTGLNTRSQETYETLK | |
| | | HEKPPQ | |

In some embodiments, the chimeric receptor comprises, from N- to C-terminus, an extracellular domain comprising an amyloid-binding peptide or a functional fragment thereof; a transmembrane domain; and a cytoplasmic domain. In some embodiments, the chimeric receptor comprises, from N- to C-terminus, an amyloid binding peptide or a functional fragment thereof, a first spacer, a CH2 domain, a second spacer, a transmembrane domain, and a cytoplasmic domain. In some embodiments, the chimeric receptor comprises, from N- to C-terminus, an N-terminal secretory leader sequence, an amyloid binding peptide or a functional fragment thereof, a first spacer, a CH2 domain, a second spacer, a transmembrane domain, and a cytoplasmic domain. In some embodiments, the amyloid binding peptide or a functional fragment thereof comprises the amino acid sequence of SEQ ID NO:1. In some embodiments, the amyloid binding peptide or a functional fragment thereof comprises the amino acid sequence of SEQ ID NO:17. In some embodiments, the cytoplasmic domain is derived from FcR. In some embodiments, the cytoplasmic domain is derived from FcR and CD19. In some embodiments, the cytoplasmic domain comprises a cytoplasmic domain II, as shown in Table 3. In some embodiments, the cytoplasmic domain comprises a cytoplasmic domain I and a cytoplasmic domain II, as shown in Table 3. In some embodiments, the chimeric receptor is a CAR-P as shown in Table 3. In some embodiments, the chimeric receptor is a peptide-based CAR as shown in Table C. In some embodiments, the chimeric receptor comprises an amino acid sequence having 80, 85, 90, 95, 97, 98, or 99% sequence identity to the sequence set forth as the CAR-P Construct-345aa in Table 3, with or without the N-terminal secretory leader sequence. In some embodiments, each component of the chimeric receptor has 80, 85, 90, 95, 97, 98, or 99% sequence identity to the corresponding component of as the CAR-P Construct-345aa in Table 3, together or separately.

In some embodiments, the chimeric receptor comprises, from N- to C-terminus, an extracellular domain comprising p5, a first spacer, an IgG2 CH2 domain, a second spacer, a transmembrane domain derived from a CD8 α chain, and a cytoplasmic domain derived from FcR. In some embodiments, the chimeric receptor comprises, from N- to C-terminus, an extracellular domain comprising p5, a first spacer, an IgG2 CH2 domain, a second spacer, a transmembrane domain derived from a CD8 α chain, and a cytoplasmic domain derived from FcR and CD19. In some embodiments, the chimeric receptor comprises, from N- to C-terminus, an N-terminal secretory leader sequence, an extracellular domain comprising p5, a first spacer, an ISG2 CH2 domain, a second spacer, a transmembrane domain derived from a CD8 α chain, and a cytoplasmic domain derived from FcR. In some embodiments, the chimeric receptor comprises, from N- to C-terminus, an N-terminal secretory leader sequence, an extracellular domain comprising p5, a first spacer, an IgG2 CH2 domain, a second spacer, a transmembrane domain derived from a CD8 α chain, and a cytoplasmic domain derived from FcR and CD19.

In some embodiments, the chimeric receptor comprises, from N- to C-terminus, an extracellular domain comprising p5+14, a first spacer, an IgG2 CH2 domain, a second spacer, a transmembrane domain derived from a CD8 α chain, and a cytoplasmic domain derived from FcR. In some embodiments, the chimeric receptor comprises, from N- to C-terminus, an extracellular domain comprising p5+14, a first spacer, an IG2 CH2 domain, a second spacer, a transmembrane domain derived from a CD8 α chain, and a cytoplasmic domain derived from FcR and CD19. In some embodiments, the chimeric receptor comprises, from N- to C-terminus, an N-terminal secretory leader sequence, an extracellular domain comprising p5+14, a first spacer, an IgG2 CH2 domain, a second spacer, a transmembrane domain derived from a CD8 α chain, and a cytoplasmic domain derived from FcR. In some embodiments, the chimeric receptor comprises, from N- to C-terminus, an N-terminal secretory leader sequence, an extracellular domain comprising p5+14, a first spacer, an ISG2 CH2 domain, a second spacer, a transmembrane domain derived from a CD8 α chain, and a cytoplasmic domain derived from FcR and CD19.

In some embodiments, the chimeric receptor comprises, from N- to C-terminus an extracellular domain comprising a p5+14 and a first spacer according to the amino acid sequence set forth in SEQ ID NO:32, and a CH2 domain according to the amino acid sequence set forth in SEQ ID NO: 33; a second spacer; a transmembrane domain; and a cytoplasmic domain. In some embodiments, the chimeric receptor comprises, from N- to C-terminus an extracellular domain comprising a p5+14 and a first spacer according to the amino acid sequence set forth in SEQ ID NO:32, and a CH2 domain according to the amino acid sequence set forth in SEQ ID NO: 33; a second spacer; a transmembrane domain derived from a CD8 α chain; and a cytoplasmic domain derived from FcR and CD19. In some embodiments, the chimeric receptor comprises, from N- to C-terminus an extracellular domain comprising a p5+14 and a first spacer according to the amino acid sequence set forth in SEQ ID NO:32, and a CH2 domain according to the amino acid sequence set forth in SEQ ID NO: 33; a second spacer; a transmembrane domain derived from a CD8 α chain; and a cytoplasmic domain derived from FcR.

In some embodiments, the chimeric receptor comprises, from N- to C-terminus an extracellular domain comprising an N-terminal secretory leader sequence according to the amino acid sequence set forth in SEQ ID NO:38, an amyloid binding peptide or a functional fragment thereof and a first spacer according to the amino acid sequence set forth in SEQ ID NO: 39, and a CH2 domain according to the amino acid sequence set forth in SEQ ID NO:33; a second spacer and a transmembrane domain according to the amino acid sequence set forth in SEQ ID NO: 40; and a cytoplasmic domain comprising a cytoplasmic domain II according to the amino acid sequence set forth in SEQ ID NO: 41 and a cytoplasmic domain I according to the amino acid sequence set forth in SEQ ID NO: 42. In some embodiments, the chimeric receptor comprises, from N- to C-terminus an extracellular domain comprising an N-terminal secretory leader sequence according to the amino acid sequence set forth in SEQ ID NO:38, an amyloid binding peptide or a functional fragment thereof and a first spacer according to the amino acid sequence set forth in SEQ ID NO: 39, and a CH2 domain according to the amino acid sequence set forth in SEQ ID NO:33; a second spacer and a transmembrane domain according to the amino acid sequence set forth in SEQ ID NO: 40; and a cytoplasmic domain comprising a cytoplasmic domain II according to the amino acid sequence set forth in SEQ ID NO: 41. In some embodiments, the chimeric receptor comprises, from N- to C-terminus an extracellular domain comprising an amyloid binding peptide or a functional fragment thereof and a first spacer according to the amino acid sequence set forth in SEQ ID NO: 39, and a CH2 domain according to the amino acid sequence set forth in SEQ ID NO:33; a second spacer and a transmembrane domain according to the amino acid sequence set forth in SEQ ID NO: 40; and a cytoplasmic domain comprising a cytoplasmic domain II according to the amino acid sequence set forth in SEQ ID NO: 41 and a cytoplasmic domain I according to the amino acid sequence set forth in SEQ ID NO: 42. In some embodiments, the chimeric receptor comprises, from N- to C-terminus an extracellular domain comprising an amyloid binding peptide or a functional fragment thereof and a first spacer according to the amino acid sequence set forth in SEQ ID NO: 39, and a CH2 domain according to the amino acid sequence set forth in SEQ ID NO:33; a second spacer and a transmembrane domain according to the amino acid sequence set forth in SEQ ID NO: 40; and a cytoplasmic domain comprising a cytoplasmic domain II according to the amino acid sequence set forth in SEQ ID NO: 41.

In some embodiments, the chimeric receptor comprises, from N- to C-terminus, an N-terminal secretory leader sequence, an extracellular domain comprising an amyloid binding peptide or a functional fragment thereof (e.g., p5 or p5-14), a first spacer, and a CH2 domain; a second spacer, a transmembrane domain derived from a CD8 α chain; and a cytoplasmic domain derived from FcR and CD19. In some embodiments, the chimeric receptor comprises the amino acid sequence of SEQ ID NO:43. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:43. In certain embodiments, the chimeric receptor comprises an amino acid sequence containing substitutions (e.g., conservative substitutions), insertions, or deletions relative to the amino acid sequence of SEQ ID NO:43, but retaining the ability to bind amyloid and activate a phagocytic cell as a chimeric receptor comprising the amino acid sequence of SEQ ID NO:43. In certain embodiments, a total of 1 to 15 amino acids have been substituted, inserted and/or deleted in SEQ ID NO:43.

In some embodiments, the chimeric receptor comprises, from N- to C-terminus, an N-terminal secretory leader sequence, an extracellular domain comprising an amyloid binding peptide or a functional fragment thereof (e.g., p5 or p5-14), a first spacer, and a CH2 domain; a second spacer; a transmembrane domain derived from a CD8 α chain; and a cytoplasmic domain derived from FcR. In some embodiments, the chimeric receptor comprises the amino acid sequence of SEQ ID NO:51. In some embodiments, the chimeric receptor comprises an amino acid sequence having at least 80%, 81% 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:51. In certain embodiments, the chimeric receptor comprises an amino acid sequence containing substitutions (e.g., conservative substitutions), insertions, or deletions relative to the amino acid sequence of SEQ ID NO:51, but retaining the ability to bind amyloid and activate a phagocytic cell as a chimeric receptor comprising the amino acid sequence of SEQ ID NO:51. In certain embodiments, a total of 1 to 15 amino acids have been substituted, inserted and/or deleted in SEQ ID NO:51.

In some embodiments, the chimeric receptor comprises, from N- to C-terminus, an extracellular domain comprising an amyloid binding peptide or a functional fragment thereof (e.g., p5 or p5-14), a first spacer, and a C2 domain; a second spacer, a transmembrane domain derived from a CD8 α chain; and a cytoplasmic domain derived from FcR and CD19. In some embodiments, the chimeric receptor comprises the amino acid sequence of SEQ ID NO:52. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:52. In certain embodiments, the chimeric receptor comprises an amino acid sequence containing substitutions (e.g., conservative substitutions), insertions, or deletions relative to the amino acid sequence of SEQ ID NO:52, but retaining the ability to bind amyloid and activate a phagocytic cell as a chimeric receptor comprising the amino acid sequence of SEQ ID NO:52. In certain embodiments, a total of 1 to 15 amino acids have been substituted, inserted and/or deleted in SEQ ID NO:52.

In some embodiments, the chimeric receptor comprises, from N- to C-terminus, an extracellular domain comprising an amyloid binding peptide or a functional fragment thereof (e.g., p5 or p5-14), a first spacer, and a CH2 domain; a second spacer, a transmembrane domain derived from a CD8 α chain; and a cytoplasmic domain derived from FcR. In some embodiments, the chimeric receptor comprises the amino acid sequence of SEQ ID NO:53. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99, or 100% sequence identity to the amino acid sequence of SEQ ID NO:53. In certain embodiments, the chimeric receptor comprises an amino acid sequence containing substitutions (e.g., conservative substitutions), insertions, or deletions relative to the amino acid sequence of SEQ ID NO:53, but retaining the ability to bind amyloid and activate a phagocytic cell as a chimeric receptor comprising the amino acid sequence of SEQ ID NO:53. In certain embodiments, a total of 1 to 15 amino acids have been substituted, inserted and/or deleted in SEQ ID NO:53.

In some embodiments, the chimeric receptor comprises, from N- to C-terminus, an extracellular domain comprising an antibody fragment or functional fragment thereof (e.g., an antibody fragment or functional fragment thereof derived from 11-1F4), a transmembrane domain, and a cytoplasmic domain. In some embodiments, the chimeric receptor comprises, from N- to C-terminus, a VL, a linker, a VH, a spacer, a transmembrane domain, and a cytoplasmic domain. In some embodiments, the chimeric receptor comprises, from N- to C-terminus, an N-terminal secretory leader sequence, a VL, a linker, a VH, a spacer, a transmembrane domain, and a cytoplasmic domain. In some embodiments, the VH comprises (a) a CDR-H1 comprising the amino acid sequence of SEQ ID NO:21, (b) a CDR-H2 comprising the amino acid sequence of SEQ ID NO:22, and (c) a CDR-H3 comprising the amino acid sequence of SEQ ID NO:23; and the VL comprises (a) a CDR-L1 comprising the amino acid sequence of SEQ ID NO:24; (b) a CDR-L2 comprising the amino acid sequence of SEQ ID NO:25; and (c) a CDR-L3 comprising the amino acid sequence of SEQ ID NO:26. In some embodiments, the cytoplasmic domain is derived from FcR. In some embodiments, the cytoplasmic domain is derived from FcR and CD19. In some embodiments, the cytoplasmic domain comprises a cytoplasmic domain IL as shown in Table 1. In some embodiments, the cytoplasmic domain comprises a cytoplasmic domain I and a cytoplasmic domain II, as shown in Table 1. In some embodiments, the chimeric receptor is an ^{11-1F4}CAR_{tandem} receptor, as shown in Table 1. In some embodiments, the chimeric receptor is an scFv-based CAR as shown in Table G.

In some embodiments, the chimeric receptor comprises, from N- to C-terminus, an extracellular domain comprising an antibody fragment derived from 11-1F4 or a functional fragment thereof, a transmembrane domain derived from a CD8 α chain, and a cytoplasmic domain derived from FcR. In some embodiments, the chimeric receptor comprises, from N- to C-terminus, an extracellular domain comprising an antibody fragment derived from 11-1F4 or functional fragment thereof, a transmembrane domain derived from a CD8 α chain, and a cytoplasmic domain derived from FcR and CD19. In some embodiments, the chimeric receptor comprises, from N- to C-terminus, a VL derived from 11-1F4, a linker, a VH derived from 11-1F4, a spacer, a transmembrane domain derived from a CD8 α chain, and a cytoplasmic domain derived from FcR. In some embodiments, the chimeric receptor comprises, from N- to C-terminus, a VL derived from 11-1F4, a linker, a VH derived from 11-1F4, a spacer, a transmembrane domain derived from a CD8 α chain, and a cytoplasmic domain derived from FcR and CD19. In some embodiments, the chimeric receptor comprises, from N- to C-terminus, an N-terminal secretory leader sequence, a VL derived from 11-1F4, a linker, a VH derived from 11-1F4, a spacer, a transmembrane domain derived from a CD8 α chain, and a cytoplasmic domain derived from FcR. In some embodiments, the chimeric receptor comprises, from N- to C-terminus, an N-terminal secretory leader sequence, a VL derived from 11-1F4, a linker, a VH derived from 11-1F4, a spacer, a transmembrane domain derived from a CD8 α chain, and a cytoplasmic domain derived from FcR and CD19.

In some embodiments, the chimeric receptor comprises, from N- to C-terminus, an N-terminal secretory leader sequence comprising the amino acid sequence set forth in SEQ ID NO:28, a VL comprising the amino acid sequence set forth in SEQ ID NO:19, an scFv linker comprising the amino acid sequence set forth in SEQ ID NO:27, a VH comprising the amino acid sequence set forth in SEQ ID NO:20, a spacer and transmembrane domain comprising the amino acid sequence set forth in SEQ ID NO:29, and a cytoplasmic domain comprising the amino acid sequence set forth in SEQ ID NO:31. In some embodiments, the chimeric receptor comprises, from N- to C-terminus, an N-terminal secretory leader sequence comprising the amino acid sequence set forth in SEQ ID NO:28, a VL comprising the amino acid sequence set forth in SEQ ID NO:19, an scFv linker comprising the amino acid sequence set forth in SEQ ID NO:27, a VH comprising the amino acid sequence set forth in SEQ ID NO:20, a spacer and transmembrane domain comprising the amino acid sequence set forth in SEQ ID NO:29, and a cytoplasmic domain comprising the amino acid sequence set forth in SEQ ID NO:31 and the amino acid sequence set forth in SEQ ID NO:30. In some embodiments, the chimeric receptor comprises, from N- to C-terminus, a VL comprising the amino acid sequence set forth in SEQ ID NO:19, an scFv linker comprising the amino acid sequence set forth in SEQ ID NO:27, a VH comprising the amino acid sequence set forth in SEQ ID NO:20, a spacer and transmembrane domain comprising the amino acid sequence set forth in SEQ ID NO:29, and a cytoplasmic domain comprising the amino acid sequence set forth in SEQ ID NO:31. In some embodiments, the chimeric receptor comprises, from N- to C-terminus, a VL comprising the amino acid sequence set forth in SEQ ID NO:19, an scFv linker comprising the amino acid sequence set forth in SEQ ID NO:27, a VH comprising the amino acid sequence set forth in SEQ ID NO:20, a spacer and transmembrane domain comprising the amino acid sequence set forth in SEQ ID NO:29, and a cytoplasmic domain comprising the amino acid sequence set forth in SEQ ID NO:31 and the amino acid sequence set forth in SEQ ID NO:30.

In some embodiments, the chimeric receptor comprises, from N- to C-terminus, an N-terminal secretory leader sequence, an extracellular domain comprising an scFv derived from 11-1F4; a spacer; a transmembrane domain derived from a CD8 α chain; and a cytoplasmic domain derived from FcR and CD19. In some embodiments, the chimeric receptor comprises the amino acid sequence of SEQ ID NO:50. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50. In certain embodiments, the chimeric receptor comprises an amino acid sequence containing substitutions (e.g., conservative substitutions), insertions, or deletions relative to the amino acid sequence of SEQ ID NO:50, but retaining the ability to bind amyloid and activate a phagocytic cell as a chimeric receptor comprising the amino acid sequence of SEQ ID NO:50. In certain embodiments, a total of 1 to 15 amino acids have been substituted, inserted and/or deleted in SEQ ID NO:50.

In some embodiments, the chimeric receptor comprises, from N- to C-terminus, an N-terminal secretory leader sequence, an extracellular domain comprising an scFv derived from 11-1F4; a spacer; a transmembrane domain derived from a CD8 α chain; and a cytoplasmic domain derived from FcR. In some embodiments, the chimeric receptor comprises the amino acid sequence of SEQ ID NO:49. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:49. In certain embodiments, the chimeric receptor comprises an amino acid sequence containing substitutions (e.g., conservative substitutions), insertions, or deletions relative to the amino acid sequence of SEQ ID NO:49, but retaining the ability to bind amyloid and activate a phagocytic cell as a chimeric receptor comprising the amino acid sequence of SEQ ID NO:49. In certain embodiments, a total of 1 to 15 amino acids have been substituted, inserted and/or deleted in SEQ ID NO:49.

In some embodiments, the chimeric receptor comprises, from N- to C-terminus, an extracellular domain comprising an scFv derived from 11-1F4; a spacer; a transmembrane domain derived from a CD8 α chain; and a cytoplasmic domain derived from FcR and CD19. In some embodiments, the chimeric receptor comprises the amino acid sequence of SEQ ID NO:55. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99, or 100% sequence identity to the amino acid sequence of SEQ ID NO:55. In certain embodiments, the chimeric receptor comprises an amino acid sequence containing substitutions (e.g., conservative substitutions), insertions, or deletions relative to the amino acid sequence of SEQ ID NO:55, but retaining the ability to bind amyloid and activate a phagocytic cell as a chimeric receptor comprising the amino acid sequence of SEQ ID NO:55. In certain embodiments, a total of 1 to 15 amino acids have been substituted, inserted and/or deleted in SEQ ID NO:55.

In some embodiments, the chimeric receptor comprises, from N- to C-terminus, an extracellular domain comprising an scFv derived from 11-1F4; a spacer; a transmembrane domain derived from a CD8 α chain; and a cytoplasmic domain derived from FcR. In some embodiments, the chimeric receptor comprises the amino acid sequence of SEQ ID NO:54. In some embodiments, the chimeric receptor comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99, or 100% sequence identity to the amino acid sequence of SEQ ID NO:54. In certain embodiments, the chimeric receptor comprises an amino acid sequence containing substitutions (e.g., conservative substitutions), insertions, or deletions relative to the amino acid sequence of SEQ ID NO:54, but retaining the ability to bind amyloid and activate a phagocytic cell as a chimeric receptor comprising the amino acid sequence of SEQ ID NO:54. In certain embodiments, a total of 1 to 15 amino acids have been substituted, inserted and/or deleted in SEQ ID NO:54.

In some embodiments, the chimeric receptors described herein bind to amyloid deposits or fibrils (e.g., human amyloid deposits or fibrils). In some embodiments, the amyloid-binding region of the chimeric receptor binds to one or more amyloidogenic peptides in amyloids. In some embodiments, amyloids bound by the amyloid-binding region of the chimeric receptor comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the amyloid-binding region of the chimeric receptor comprise amyloidogenic forms of immunoglobulin heavy chain (AH), β₂-microglobulin (Aβ₂M), transthyretin variants (ATI), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, Alλ1 other amyloidogenic peptides. The amyloidogenic peptides bound by the amyloid-binding region of the chimeric receptor can be a protein, a protein fragment, or a protein domain. In some embodiments, the amyloid deposits or amyloid fibrils comprise recombinant amyloidogenic proteins. In some embodiments, the amyloids are part of the pathology of a disease.

In some embodiments, the cytoplasmic domains of the chimeric receptors described herein comprise signaling domains that, when activated, activate a phagocytic cell (e.g., a macrophage). In some embodiments, the signaling domains of the cytoplasmic domains are activated upon binding of the chimeric receptor to amyloid deposits or fibrils, as described above. In some embodiments, activation of the phagocytic cell promotes phagocytosis of the amyloid deposits or fibrils.

In some embodiments, the chimeric receptor is conjugated to a detectable label. In some embodiments, the detectable label is selected from the group consisting of radionuclides (e.g., I-¹²⁵, I-¹²³, I-¹³¹, Zr-⁸⁹, Tc-^{99m}, Cu-⁶⁴, Br-⁷⁶, F-¹⁸); enzymes (horse radish peroxidase); biotin; and fluorophores, etc. Any means known in the art for detectably labeling a protein can be used and/or adapted for use with the methods described herein. For example, the chimeric receptor can be radiolabeled with a radioisotope, or labeled with a fluorescent tag or a chemiluminescent tag Example radioisotopes include, for example, ¹⁸F, ¹¹¹In, ^{99m}Tc, and ¹²³I, and ¹²⁵I. These and other radioisotopes can be attached to the chimeric receptor using well known chemistry that may or not involve the use of a chelating agent, such as DTPA or DOTA covalently linked to the chimeric receptor, for example. Example fluorescent or chemiluminescent tags include fluorescein, Texas red, rhodamine, Alexa dyes, and luciferase that can be conjugated to the chimeric receptor by reaction with lysine, cysteine, glutamic acid, and aspartic acid side chains. In one example embodiment, the label is detected using a fluorescent microplate reader, or fluorimeter, using the excitation and emission wavelengths appropriate for the tag that is used. Radioactive labels can be detected, for example, using a gamma or scintillation counter depending on the type of radioactive emission and by using energy windows suitable for the accurate detection of the specific radionuclide. However, any other suitable technique for detection of radioisotopes can also be used to detect the label. In some embodiments, the detectable label is ¹²⁵I. In some embodiments, the chimeric receptor is fused to a fluorescent protein. In some embodiments, the chimeric receptor is fused to GFP.

Also provided herein are pharmaceutical compositions comprising any of the chimeric receptors described herein. In some embodiments, the pharmaceutical composition further comprises a pharmaceutically acceptable carrier.

### III. Nucleic Acids, Vectors, Host Cells, and Methods of Making Chimeric Receptors

A. Nucleic Acids Encoding Chimeric Receptors

Provided herein are nucleic acid(s) encoding chimeric receptors that bind amyloid. In some embodiments, the nucleic acid encodes any one of the chimeric receptors described herein. In some embodiments, the nucleic acid encodes a chimeric receptor comprising a cytoplasmic domain, wherein the cytoplasmic domain comprises a signaling domain of a receptor that when activated activates a macrophage; a transmembrane domain; and an extracellular domain, wherein the extracellular domain comprises an amyloid binding region. In some embodiments, the nucleic acid encodes a chimeric receptor comprising, from N- to C-terminus, an extracellular domain, a transmembrane domain, and a cytoplasmic domain.

In some embodiments, the nucleic acid encodes a chimeric receptor, wherein the chimeric receptor comprises an extracellular domain comprising an amyloid-binding region, wherein the amyloid binding region comprises an amyloid-binding peptide or functional fragment thereof. The amyloid binding region comprises an amyloid-binding peptide or functional fragment thereof may be any one of the amyloid binding regions comprising an amyloid-binding peptide or functional fragment thereof as described herein.

In some embodiments, the nucleic acid encodes a chimeric receptor comprising, from N- to C-terminus, an extracellular domain comprising an amyloid-binding peptide or a functional fragment thereof, a transmembrane domain, and a cytoplasmic domain. In some embodiments, the nucleic acid encodes a chimeric receptor comprising, from N- to C-terminus, an amyloid binding peptide or a functional fragment thereof, a first spacer, a CH2 domain, a second spacer, a transmembrane domain, and a cytoplasmic domain. In some embodiments, the nucleic acid encodes a chimeric receptor comprising, from N- to C-terminus, an N-terminal secretory leader sequence, an amyloid binding peptide or a functional fragment thereof, a first spacer, a CH2 domain, a second spacer, a transmembrane domain, and a cytoplasmic domain. In some embodiments, the amyloid-binding peptide or a functional fragment thereof comprises the amino acid sequence of SEQ ID NO:1. In some embodiments, the amyloid-binding peptide or a functional fragment thereof comprises the amino acid sequence of SEQ ID NO:17. In some embodiments, the cytoplasmic domain comprises a cytoplasmic domain II, as shown in Table 3. In some embodiments, the cytoplasmic domain comprises a cytoplasmic domain I and a cytoplasmic domain II, as shown in Table 3. In some embodiments, the nucleic acid encodes a CAR-P as shown in Table 3. In some embodiments, the nucleic acid encodes a chimeric receptor comprising an amino acid sequence having 80, 85, 90, 95, 97, 98, or 99% sequence identity to the sequence set forth as the CAR-P Construct-345aa in Table 3, with or without the secretory leader sequence. In some embodiments, each component of the chimeric receptor encoded by the nucleic acid has 80, 85, 90, 95, 97, 98, or 99% sequence identity to the corresponding component of as the CAR-P Construct-345aa in Table 3, together or separately.

In some embodiments, the nucleic acid encodes a chimeric receptor comprising the amino acid sequence of SEQ ID NO:43. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:43. In certain embodiments, the nucleic acid encodes a chimeric receptor comprising an amino acid sequence containing substitutions (e.g., conservative substitutions), insertions, or deletions relative to the amino acid sequence of SEQ ID NO:43, but retaining the ability to bind amyloid and activate a phagocytic cell as a chimeric receptor comprising the amino acid sequence of SEQ ID NO:43. In certain embodiments, a total of 1 to 15 amino acids have been substituted, inserted and/or deleted in SEQ ID NO:43.

In some embodiments, the nucleic acid encodes a chimeric receptor comprising the amino acid sequence of SEQ ID NO:51. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:51. In certain embodiments, the nucleic acid encodes a chimeric receptor comprising an amino acid sequence containing substitutions (e.g., conservative substitutions), insertions, or deletions relative to the amino acid sequence of SEQ ID NO:51, but retaining the ability to bind amyloid and activate a phagocytic cell as a chimeric receptor comprising the amino acid sequence of SEQ ID NO:51. In certain embodiments, a total of 1 to 15 amino acids have been substituted, inserted and/or deleted in SEQ ID NO:51

In some embodiments, the nucleic acid encodes a chimeric receptor comprising the amino acid sequence of SEQ ID NO:52. In some embodiments, the nucleic acid encodes a chimeric receptor comprising an amino acid sequence having at least 80%, 81%, 82%, 83%, 84%, 85%, 86%, 87/s, 88%, 89%, 90%, 91%, 92%, 93%, 94%, 95%, 96%, 97%, 98%, 99%, or 100% sequence identity to the amino acid sequence of SEQ ID NO:52. In certain embodiments, the nucleic acid encodes a chimeric receptor comprising an amino acid sequence containing substitutions (e.g., conservative substitutions), insertions, or deletions relative to the amino acid sequence of SEQ ID NO:52, but retaining the ability to bind amyloid and activate a phagocytic cell as a chimeric receptor comprising the amino acid sequence of SEQ ID NO:52. In certain embodiments, a total of t to 15 amino acids have been substituted, inserted and/or deleted in SEQ ID NO:52.

In some embodiments, the nucleic acid encodes a chimeric receptor comprising the amino acid sequence of SEQ ID NO:53. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:53. In certain embodiments, the nucleic acid encodes a chimeric receptor comprising an amino acid sequence containing substitutions (e.g., conservative substitutions), insertions, or deletions relative to the amino acid sequence of SEQ ID NO:53, but retaining the ability to bind amyloid and activate a phagocytic cell as a chimeric receptor comprising the amino acid sequence of SEQ ID NO:53. In certain embodiments, a total of 1 to 15 amino acids have been substituted, inserted and/or deleted in SEQ ID NO:53.

In some embodiments, the nucleic acid encodes a chimeric receptor, wherein the chimeric receptor comprises an extracellular domain comprising an amyloid-binding region, wherein the amyloid binding region comprises an 11-1F4 antibody fragment.

In some embodiments, the nucleic acid encodes a chimeric receptor comprising, from N- to C-terminus, an extracellular domain comprising an 11-1F4 antibody fragment, a transmembrane domain, and a cytoplasmic domain. In some embodiments, the nucleic acid encodes a chimeric receptor comprising, from N- to C-terminus, a VL, a linker, a VH, a spacer, a transmembrane domain, and a cytoplasmic domain. In some embodiments, the nucleic acid encodes a chimeric receptor comprising, from N- to C-terminus, an N-terminal secretory leader sequence, a VL, a linker, a VH, a spacer, a transmembrane domain, and a cytoplasmic domain. In some embodiments, the VH comprises (a) a CDR-H1 comprising the amino acid sequence of SEQ ID NO:21, (b) a CDR-H2 comprising the amino acid sequence of SEQ ID NO:22, and (c) a CDR-H3 comprising the amino acid sequence of SEQ ID NO:23; and the VL comprises (a) a CDR-L1 comprising the amino acid sequence of SEQ ID NO:24; (b) a CDR-L2 comprising the amino acid sequence of SEQ ID NO:25; and (c) a CDR-L3 comprising the amino acid sequence of SEQ ID NO:26. In some embodiments, the cytoplasmic domain comprises a cytoplasmic domain II, as shown in Table 1. In some embodiments, the cytoplasmic domain comprises a cytoplasmic domain I and a cytoplasmic domain II, as shown in Table 1. In some embodiments, the nucleic acid encodes an ^{11-1F4}CAR_{tandem} receptor, as shown in Table 1.

In some embodiments, the nucleic acid encodes a chimeric receptor comprising the amino acid sequence of SEQ ID NO:49. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:49. In certain embodiments, the nucleic acid encodes a chimeric receptor comprising an amino acid sequence containing substitutions (e.g., conservative substitutions), insertions, or deletions relative to the amino acid sequence of SEQ ID NO:49, but retaining the ability to bind amyloid and activate a phagocytic cell as a chimeric receptor comprising the amino acid sequence of SEQ ID NO:49. In certain embodiments, a total of 1 to 15 amino acids have been substituted, inserted and/or deleted in SEQ ID NO:49.

In some embodiments, the nucleic acid encodes a chimeric receptor comprising the amino acid sequence of SEQ ID NO:50. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50. In certain embodiments, the nucleic acid encodes a chimeric receptor comprising an amino acid sequence containing substitutions (e.g., conservative substitutions), insertions, or deletions relative to the amino acid sequence of SEQ ID NO:50, but retaining the ability to bind amyloid and activate a phagocytic cell as a chimeric receptor comprising the amino acid sequence of SEQ ID NO:50. In certain embodiments, a total of 1 to 15 amino acids have been substituted, inserted and/or deleted in SEQ ID NO:50.

In some embodiments, the nucleic acid encodes a chimeric receptor comprising the amino acid sequence of SEQ ID NO:54. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:54. In certain embodiments, the nucleic acid encodes a chimeric receptor comprising an amino acid sequence containing substitutions (e.g., conservative substitutions), insertions, or deletions relative to the amino acid sequence of SEQ ID NO:54, but retaining the ability to bind amyloid and activate a phagocytic cell as a chimeric receptor comprising the amino acid sequence of SEQ ID NO:54. In certain embodiments, a total of t to 15 amino acids have been substituted, inserted and/or deleted in SEQ ID NO:54.

In some embodiments, the nucleic acid encodes a chimeric receptor comprising the amino acid sequence of SEQ ID NO:55. In some embodiments, the nucleic acid encodes a chimeric receptor comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:55. In certain embodiments, the nucleic acid encodes a chimeric receptor comprising an amino acid sequence containing substitutions (e.g., conservative substitutions), insertions, or deletions relative to the amino acid sequence of SEQ ID NO:55, but retaining the ability to bind amyloid and activate a phagocytic cell as a chimeric receptor comprising the amino acid sequence of SEQ ID NO:55. In certain embodiments, a total of 1 to 15 amino acids have been substituted, inserted and/or deleted in SEQ ID NO:55.

B. Vectors, Host Cells

In some embodiments, the nucleic acid provided herein are in one or more vectors. For example, in some embodiments, provided herein is a vector comprising a nucleic acid encoding a chimeric receptor. In some embodiments, the vector comprises the nucleic acid(s) encoding a chimeric receptor of the present disclosure.

In some embodiments, the vector is a viral vector. In some embodiments, the vector is a retroviral vector. In some embodiments, the vector is a gamma retroviral vector. In some embodiments, the vector is a lentiviral vector. In some embodiments, the vector is an adenoviral vector. In some embodiments, the vector is an adeno-associated viral (AAV) vector.

In some embodiments, the vector is a pEF-ENTR A vector.

In some embodiments, the vector encodes multiple gene products. In some embodiments, the vector is a bicistronic vector. In some embodiments, the vector comprises a nucleic acid that encodes a second protein product, e.g., a fluorescent protein such as green fluorescent protein (GFP).

In some embodiments, the vector is a transposase vector. In some embodiments, the vector is a piggyBac vector.

In some embodiments, the vector comprises a promoter. In some embodiments, the nucleic acid encoding the chimeric receptor is operably linked to the promoter. In some embodiments, the promoter is an inducible promoter. In some embodiments, the promoter is a ubiquitously expressed promoter. In some embodiments, the vector comprises an EF1-a promoter. In some embodiments, the nucleic acid encoding the chimeric receptor is operably linked to the EF1-a promoter.

In some embodiments, the vector comprises a macrophage-specific regulatory element, e.g., a macrophage-specific promoter. In some embodiments, the nucleic acid encoding the chimeric receptor is operably linked to the macrophage-specific regulatory element. In some embodiments, the nucleic acid encoding the chimeric receptor is operably linked to a promoter that drives expression in macrophages.

Also provided herein is a host cell comprising a nucleic acid encoding any of the chimeric receptors described herein. In some embodiments, the host cell comprising a vector comprising nucleic acid(s) encoding a chimeric receptor of the present disclosure. In some embodiments, vertebrate cells may be used as host cells. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### IV. Engineered Cells Comprising Chimeric Receptors

Provided herein are engineered cells comprising the chimeric receptors of the present disclosure. In some embodiments, an engineered cell comprising any one of the chimeric receptors described herein is provided. In some embodiments, the engineered cell is a phagocytic cell. In some embodiments, the engineered cell is a monocyte, a macrophage, or a dendritic cell. In some embodiments, the engineered cell is a macrophage. In some embodiments, engineered the cell is a murine macrophage. In some embodiments, the engineered cell is a RAW264.7 cell (e.g., ATCC TIB-71). In some embodiments, the engineered cell is a human macrophage.

In some embodiments, the engineered cell expresses a chimeric receptor of the present disclosure. In some embodiments, the engineered cell expresses the chimeric receptor from a nucleic acid encoding the chimeric receptor (e.g., any one of the nucleic acids described herein). In some embodiments, the engineered cell expresses the chimeric receptor from a vector (e.g., any one of the vectors described herein). In some embodiments, the nucleic acid and/or vector is integrated into the genome of the engineered cell. In some embodiments, the chimeric receptor is transiently expressed in the engineered cell. In some embodiments, the engineered cell expresses the chimeric receptor from an mRNA encoding the chimeric receptor. In some embodiments, the engineered cell comprises the chimeric receptor at the plasma membrane of the engineered cell.

In some embodiments, a cell is obtained from a subject, and the cell is engineered by introduction of a chimeric receptor of the present disclosure. Non-limiting examples of subjects include humans, dogs, cats, mice, rats, and transgenic species thereof. In some embodiments, the subject is a human. The cells can be obtained from a number of sources, including peripheral blood mononuclear cells, bone marrow, lymph node tissue, spleen tissue, umbilical cord, and tumors. In certain embodiments, any number of monocyte, macrophage, dendritic cell or progenitor cell lines available in the art, may be used. In certain embodiments, the cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the monocytes, macrophages and/or dendritic cells can be further isolated by positive or negative selection techniques.

The cells so isolated can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of a monocyte, macrophage and/or dendritic cell population by negative selection can be accomplished using a combination of antibodies directed to surface markers unique to the negatively selected cells. In some embodiments, enrichment is performed by cell sorting and/or selection via negative magnetic immunoadherence or flow cytometry that uses a cocktail of monoclonal antibodies directed to cell surface markers present on the cells negatively selected. For example, enrichment of a cell population for monocytes, macrophages and/or dendritic cells by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/mi is used. In further embodiments, concentrations of 125 or 150 million cells/ml can be used. The use of high concentrations of cells can result in increased cell yield, cell activation, and cell expansion.

In some embodiments, a population of cells comprising the cells (e.g., engineered monocytes, macrophages, and/or dendritic cells) of the present invention is provided. Examples of a population of cells include, but are not limited to, engineered cells derived from peripheral blood mononuclear cells, cord blood cells, a purified population of monocytes, macrophages, or dendritic cells, and a cell line. In another embodiment, peripheral blood mononuclear cells comprise the population of monocytes, macrophages, or dendritic cells. In some embodiments, a population of purified cells comprising the population of engineered monocytes, macrophages, or dendritic cells is provided.

In some embodiments, the engineered cell has upregulated M1 markers and downregulated M2 markers. For example, at least one M1 marker, such as HLA DR, CD86, CD80, and PDL1, is upregulated in the engineered cell. In another example, at least one M2 marker, such as CD206, CD163, is downregulated in the engineered cell. In one embodiment, the engineered cell has at least one upregulated M1 marker and at least one downregulated M2 marker.

In some embodiments, the engineered cell is an immunoregulatory cell. Immunoregulatory cells include T-cells, such as CD4 T-cells (Helper T-cells), CD8 T-cells (Cytotoxic T-cells, CTLs) and memory T cells or memory stem cell T cells. In another embodiment, T-cells include Natural Killer T-cells (NK T-cells).

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

NK cells mediate anti-tumor effects without the risk of GvHD and are short-lived relative to T-cells. Accordingly, NK cells would be exhausted shortly after destroying cancer cells, decreasing the need for an inducible suicide gene on CAR constructs that would ablate the modified cells.

The engineered cells may be obtained from peripheral blood, cord blood, bone marrow, tumor infiltrating lymphocytes, lymph node tissue, or thymus tissue. The engineered cells may include placental cells, embryonic stem cells, induced pluripotent stem cells, or hematopoietic stem cells. The engineered cells may be obtained from humans, monkeys, chimpanzees, dogs, cats, mice, rats, and transgenic species thereof. The engineered cells may be obtained from established cell lines.

The above cells may be obtained by any known means. The engineered cells may be autologous, syngeneic, allogeneic, or xenogeneic to the recipient of the engineered cells.

The term “autologous” refer to any material derived from the same individual to whom it is later to be re-introduced into the individual. The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenic ally.

In some embodiments, targeted effector activity in the engineered cell is enhanced by inhibition of either CD47 or SIRPα activity. CD47 and/or SIRPα activity may be inhibited by treating the cell with an anti-CD47 or anti-SIRPα antibody. Alternatively, CD47 or SIRPα activity may be inhibited by any method known to those skilled in the art.

In some embodiments, binding of the engineered cell comprising a chimeric receptor of the present disclosure to amyloid (e.g., via the binding of the amyloid binding region to amyloid) promotes the phagocytosis of human amyloid fibrils. In some embodiments, the engineered cell comprising a chimeric receptor opsonizes human amyloid fibrils. In some embodiments, the cell comprising a chimeric receptor opsonizes rVλ6Wil fibrils. In some embodiments, contacting human amyloid fibrils with an engineered cell comprising a chimeric receptor of the present disclosure promotes the uptake of the human amyloid fibrils by the cell. In some embodiments, contacting human amyloid fibrils with an engineered cell comprising a chimeric receptor of the present disclosure promotes the opsonization of the human amyloid fibrils. In some embodiments, the engineered cell comprising a chimeric receptor phagocytoses amyloid.

Also provided herein are methods of generating an engineered cell comprising a chimeric receptor (e.g., any one of the chimeric receptor described herein) CAR-expressing cells may be generated by using standard transfection or retroviral transduction of the effector cells, e.g., T-cells, with cDNA encoding the CAR. The CAR protein is then presented on the plasma cell membrane. This molecular biology technology is known in the art, and is generally associated with the development of tumor cell-directed CAR-T T-cell lymphocytes (see, e.g., Chavez, J. C. and F. L. Locke, Best Pract Res Clin Haematol, 2018. 31(2): p. 135-146; Cummins, K. D. and S. Gill, Leuk Lymphoma, 2018. 59(7): p. 1539-1553; Filley, A. C., et al., Front Oncol, 2018. 8: p. 453; Genta, S., et al., Expert Opin Biol Ther, 2018. 18(4): p. 359-367; Ghione, P., et al., Curr Hematol Malig Rep, 2018. 13(6): p. 494-506; Guo, Y., et al., Protein Cell, 2018. 9(6): p. 516-526).

In some embodiments, a polynucleotide (such as a vector) comprising the CAR is introduced into a cell by any known means. In some embodiments, the polynucleotide is introduced using transfection or transduction. In some embodiments, the polynucleotide is a viral vector.

Once the polynucleotide described above is introduced into the cell to provide an engineered cell, the engineered cells are expanded. The engineered cells containing the polynucleotide described above are expanded by any known means.

The expanded cells are isolated by any known means to provide isolated engineered cells according to the present disclosure.

### V. Methods of Treatment, Methods of Removing Amyloid

In some embodiments, provided herein are methods for removing amyloid (e.g., removing an amyloid deposit). In some embodiments, the method comprises contacting an amyloid deposit with any one of the chimeric receptors described herein. In some embodiments, the method comprises contacting an amyloid deposit with any one of the engineered cells comprising a chimeric receptor described herein. In some embodiments, the amyloid is AA, AL, AK, ATTR, Aß2M, Wild type TTR, AApoAI, AApoAII, AGel, ALys, ALect2, Afib, ACys. ACal, AMedin, AIAPP, APro, AIns, APrP, or Aβ. In some embodiments, amyloids contacted by the chimeric receptor comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA). In other embodiments, the amyloids contacted by the chimeric receptor comprise amyloidogenic forms of immunoglobulin heavy chain (AH), β₂-microglobulin (Aβ₂M), transthyretin variants (ATTR), apolipoprotein AI(AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, Alλ1 other amyloidogenic peptides. The amyloidogenic peptides contacted by the chimeric receptor can be a protein, a protein fragment, or a protein domain. In some embodiments, the amyloid comprises recombinant amyloidogenic proteins. In some embodiments, the amyloid is part of the pathology of a disease. In some embodiments, the amyloid-binding region of the chimeric receptor has binding affinity to the amyloid. In some embodiments, contacting the amyloid deposit with the chimeric receptor results in at least partial clearance of the amyloid. In some embodiments, the chimeric receptor is provided in the form of an engineered cell comprising the chimeric receptor, as described herein.

In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis. AL amyloidosis, AH amyloidosis, Aβ amyloidosis, ATTR amyloidosis, ALect2 amyloidosis, and IAPP amyloidosis of type II diabetes, Alzheimer's disease. Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer.

Also provided herein are methods of treating a subject comprising administering to the subject any one of the chimeric receptors described herein. In some embodiments, a cell (e.g., a phagocytic cell) comprising the chimeric receptor is administered. In some embodiments, a macrophage comprising the chimeric receptor is administered. In some embodiments, a monocyte comprising the chimeric receptor is administered.

In certain example embodiments, provided herein are methods of treating a subject having amyloidosis. For example, an effective amount of a cell comprising a chimeric receptor as described herein is administered to a subject, thereby treating the subject or allowing imaging of the amyloid deposits. In certain example aspects, provided is a method for clearing amyloid deposits in a subject. The method includes, for example, selecting a subject with amyloidosis and administering to the subject an effective amount of an engineered cell comprising a chimeric receptor as described herein. The engineered cells comprising a chimeric receptor include, for example, engineered cells comprising a chimeric receptor comprising an amyloid-binding peptide or functional fragment thereof, or engineered cells comprising a chimeric receptor comprising an amyloid-binding regions derived from an antibody that binds amyloid. Administration of the engineered cell comprising a chimeric receptor thereby results in clearance of the amyloid and hence treatment of the subject.

In some embodiments, the method of treating a subject having amyloidosis and/or the method of removing amyloid comprises administering a dose of engineered cells comprising a chimeric receptor to a subject in need thereof (e.g., a human having amyloidosis). In some embodiments, a therapeutically effective dose of engineered cells comprising a chimeric receptor is administered.

In some embodiments, engineered cells comprising a chimeric receptor are administered as (a) single infusion or (b) multiple infusions (e.g., a single dose split into multiple infusions).

In some embodiments, a dose of engineered cells comprising a chimeric receptor includes about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg. In embodiments, the dose of engineered cells comprising a chimeric receptor comprises about 0.6×10⁶ cells/kg, to about 2×10⁷ cells/kg. In particular embodiments, a dose of engineered cells comprising a chimeric receptor includes about 2×10⁵, 1×10⁶, 1.1×10⁶, 2×10⁶, 3×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of engineered cells comprising a chimeric receptor comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg.

In some embodiments, a dose of engineered cells comprising a chimeric receptor comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises at least about 1×10⁶ 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises about 1.1×10⁶-1.8×10⁷ cells/kg. In some embodiments, a dose of engineered cells comprising a chimeric receptor comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of engineered cells comprising a chimeric receptor comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of engineered cells comprising a chimeric receptor comprises up to about 1×10⁷, 2×10⁷, 5×10⁷, 1×10⁸, 2×108, 5×10⁸, 1×10⁹, 2×10⁹, or 5×10⁹ cells.

The engineered cells comprising a chimeric receptor can be administered by using infusion techniques that are commonly known in immunotherapy (see, e.g., Rosenberg et al., New Eng. J. of Med. 319:1676, 1988). In some embodiments, the administration of the engineered cells comprising a chimeric receptor to the subject may be carried out in any convenient manner, including by aerosol inhalation, injection, ingestion, transfusion, implantation or transplantation. The compositions described herein may be administered to a patient trans-arterially, subcutaneously, intradermally, intratumorally, intranodally, intramedullary, intramuscularly, by intravenous (i.v.) injection, or intraperitoneally. In one aspect, the engineered cells comprising a chimeric receptor are administered to a patient by intradermal or subcutaneous injection. In one aspect, engineered cells comprising a chimeric receptor of the present invention are administered by i.v. injection. The compositions of the cells comprising a chimeric receptor may be injected directly into a disease site, e.g., a site in the body with amyloid deposits.

In some embodiments, the chimeric receptor is introduced into cells (e.g., macrophages), and the subject (e.g., a human) receives an initial administration of engineered cells comprising a chimeric receptor, and one or more subsequent administrations of the engineered cells comprising a chimeric receptor, wherein the one or more subsequent administrations are administered less than 15 days, e.g., 14, 13, 12, 11, 10, 9, 8, 7, 6, 5, 4, 3, or 2 days after the previous administration. In some embodiments, more than one administration of the engineered cells comprising a chimeric receptor are administered to the subject per week, e.g., 2, 3, or 4 administrations of the cells engineered comprising a chimeric receptor are administered per week. In some embodiments, the subject receives more than one administration of the engineered cells comprising a chimeric receptor per week (e.g., 2, 3 or 4 administrations per week)(also referred to herein as a “cycle”), followed by a week of no engineered cell comprising a chimeric receptor administrations, and then one or more additional administration of the engineered cells comprising a chimeric receptor (e.g., more than one administration of the engineered cells comprising a chimeric receptor per week) is administered to the subject. In some embodiments, the subject receives more than one cycle of engineered cells comprising a chimeric receptor, and the time between each cycle is less than 10, 9, 8, 7, 6, 5, 4, or 3 days. In some embodiments, the engineered cells comprising a chimeric receptor are administered every other day for 3 administrations per week. In some embodiments, the engineered cells comprising a chimeric receptor are administered for at least two, three, four, five, six, seven, eight or more weeks.

In some embodiments, the cells comprising a chimeric receptor bind amyloid deposits in an individual. In some embodiments, the amyloid deposits may contribute to the pathology of a disease. In other embodiments, the amyloid deposits may be indicative of amyloidosis or an amyloid-related disease in an individual. In some embodiments, the cells comprising a chimeric receptor bind to amyloids in an individual with an amyloidosis. In some embodiments, the amyloidosis is localized to a specific tissue or organ system, such as the liver, the heart, or the central nervous system. In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis, AL amyloidosis, AH amyloidosis, Aβ amyloidosis, ATTR amyloidosis, ALect2 amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy. Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer. In some embodiments, the engineered cells comprising a chimeric receptor bind to amyloids associated with normal aging. In other embodiments, the engineered cells comprising a chimeric receptor are used in the diagnosis, treatment, or prognosis of an amyloidosis or amyloid-related disease in a subject.

In certain example embodiments, provided is a method for both diagnosing and treating a subject suffering from amyloidosis. Such method includes administering to the subject detectably-labeled engineered cells comprising a chimeric receptor and, based on administering the labeled engineered cells, determining that the subject is suffering from an amyloidosis. An effective amount of an amyloid treatment can then be administered to the subject. For example, an effective amount of one or more engineered cells comprising a chimeric receptor can be administered.

In some embodiments, the subject is a mammal such as primate, bovine, rodent, or pig. In some embodiments, the subject is a human.

### Embodiments

Various embodiments of the chimeric receptors, nucleic acids, vectors, host cells, engineered cells comprising chimeric receptors, and methods provided herein are included in the following non-limiting list of embodiments.

Embodiment 1. A chimeric receptor, the chimeric receptor comprising:
a cytoplasmic domain, wherein the cytoplasmic domain comprises a signaling domain of a receptor that when activated activates a macrophage;
a transmembrane domain, and
an extracellular domain, wherein the extracellular domain comprises and amyloid binding region.
Embodiment 2. The chimeric receptor of embodiment 1, wherein the amyloid binding region comprises an amyloid binding peptide or functional fragment thereof as set forth in Table A.
Embodiment 3. The chimeric receptor of embodiment 2, wherein the amyloid binding peptide or functional fragment thereof is joined directly or indirectly to a CH2 domain or fragment thereof.
Embodiment 4. The chimeric receptor of embodiment 3, wherein the CH2 domain comprises an amino acid sequence having at least 80, 85, 90, 95, 97, 98, or 99% sequence identity to the CH2 domain sequence set forth in Table 2 or Table 3.
Embodiment 5. The chimeric receptor of embodiment 1, wherein the amyloid binding region comprises an 11-1F4 antibody fragment.
Embodiment 6. The chimeric receptor of em 5, wherein the 11-1F4 antibody fragment comprises an amino acid sequence having at least 80, 85, 90, 95, 97, 98, or 99% sequence identity to the 11-1F4 VL sequence set forth in Table 1.
Embodiment 7. The chimeric receptor of embodiments 5 or 6, wherein the 11-1F4 antibody fragment is humanized.
Embodiment 8. The chimeric receptor of any of embodiments 1-7, wherein the cytoplasmic domain comprises a cytoplasmic domain I, cytoplasmic domain II, or functional fragment thereof.
Embodiment 9. The chimeric receptor of embodiment 8, wherein the cytoplasmic domain comprises an amino acid sequence having at least 80, 85, 90, 95, 97, 98, or 99% sequence identity to the sequence of the cytoplasmic domain I or cytoplasmic domain II set forth in Table 1 or Table 3.
Embodiment 10. The chimeric receptor of any of embodiments 1-9, wherein binding of an amyloid to the extracellular domain activates the cytoplasmic domain of the chimeric receptor.
Embodiment 11. A method for removing an amyloid, comprising contacting an amyloid deposit with the chimeric receptor of any of embodiments 1-10.
Embodiment 12. The method of embodiment 11, wherein the amyloid is AA, AL, AH, ATTR, Aß2M, Wild type TTR, AApoAI, AApoAII, AGel, ALys, ALect2, Afib, ACys, ACal, AMedin, AIAPP, APro, AIns, APrP, or Aβ.
Embodiment 13. The method of embodiment 12, wherein the amyloid binding region of the chimeric receptor has binding affinity to the amyloid.
Embodiment 14. The method of any of embodiments 11-13, wherein contacting the amyloid deposit with the chimeric receptor results in at least partial clearance of the amyloid.
Embodiment 15. A method of treating a subject comprising administering to the subject the chimeric receptor of any of embodiments 1-10.
Embodiment 16. The method of embodiment 15, wherein administering to the subject the chimeric receptor comprises administering a macrophage or monocyte expressing the chimeric receptor.
Embodiment 17. The chimeric receptor of embodiment 1 or the method of any of embodiments 11-16, wherein the receptor has 80, 85, 90, 95, 97, 98, or 99% sequence identity to the sequence set forth as the CAR-P Construct-345aa in Table 3, with or without the secretory leader sequence.
Embodiment 18. The chimeric receptor of embodiment 1 or the method of any of embodiments 11-16, wherein each component of the receptor has 80, 85, 90, 95. 97, 98, or 99% sequence identity to the corresponding component of as the CAR-P Construct-345aa in Table 3, together or separately.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter. The attached figures are meant to be considered as integral parts of the specification and description of the disclosure.

As used herein, the following abbreviations apply: ATTR (age-dependent transthyretin-associated); AL (amyloidosis); CAR (chimeric antigen receptor); CAR-P (chimeric antigen receptor-phagocytic); ELISA (enzyme-linked immunosorbent assay); FcR (Fc receptor); mAb (monoclonal antibody); mos (months); Mφ (macrophages); SAP (serum amyloid P); SC (subcutaneously); and scFv (single-chain variable fragment).

### Example 1. Receptors for AL Amyloidosis

The following example describes amyloid binding regions derived from the amyloid-reactive monoclonal antibody 11-1F4 and amyloid-targeting peptides.

11-1F4 Monoclonal Antibody

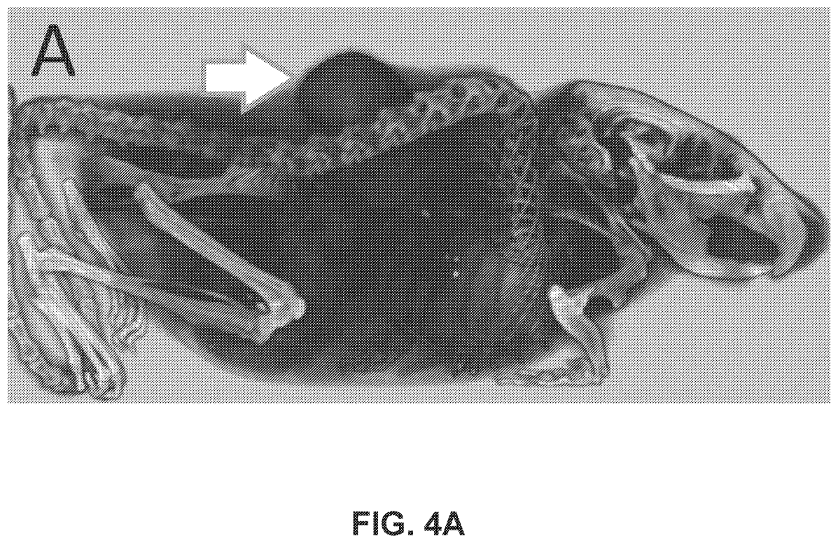

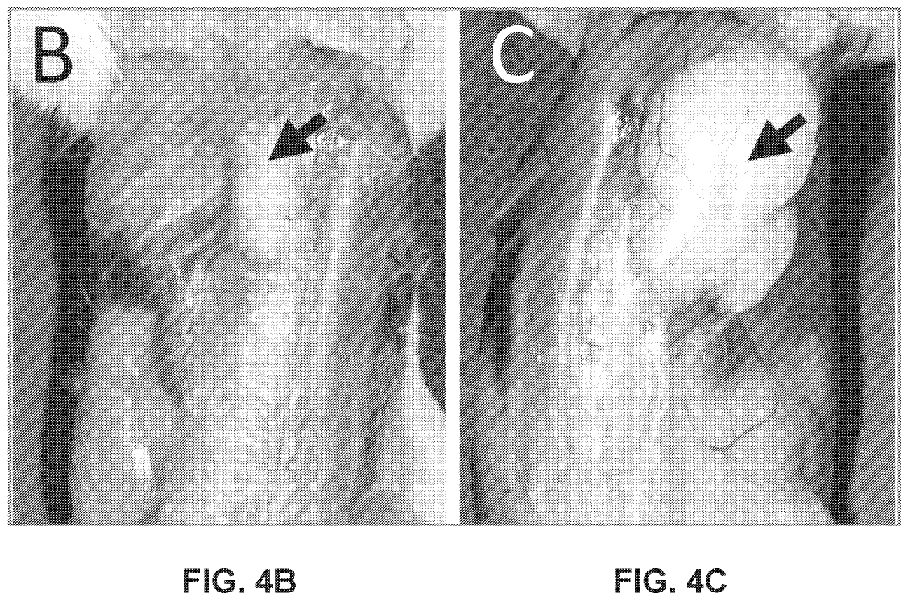

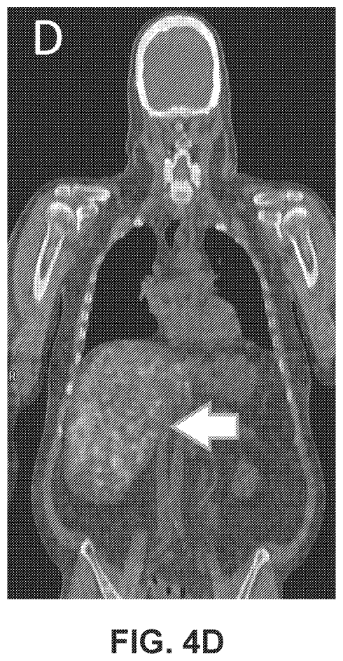

The amyloid-reactive monoclonal antibody (mAb) 11-1F4 has been generated and characterized as a therapeutic (Hrncic, R., et al., Am J Pathol, 2000. 157(4): p. 1239-46; O'Nuallain, B., et al., Amyloid and Amyloidosis: Proceedings of the Xth International Symposium on Amyloidosis. 2005. Tours, France: CRC Press; O'Nuallain, B., et al., Biochemistry, 2007. 46(5): p. 1240-7; Wall, J. S., et al., J Nucl Med, 2006. 47(12): p. 2016-2024). The mAb bound neo-epitopes present on AL amyloid fibrils, but did not avidly bind non-fibrillar LC proteins present in the circulation of AL patients. The reactivity of 11-1F4 with human AL amyloid extracts, implanted subcutaneously (SC) in immunocompromised mice, was readily evidenced by small animal SPECT/CT imaging (FIG. 4A; Wall, J. S., et al., J Nucl Med, 2006. 47(12): p. 2016-202). No off-target binding was observed in amyloid-free tissues. Treatment of mice bearing SC human AL amyloidomas with 11-1F4 (at 5 mg/Kg) expedited dissolution of the mass (FIG. 4B, FIG. 4C) in a process that required recruitment of phagocytic Mφ and neutrophils (Hrncic, R., et al., Am J Pathol, 2000. 157(4): p. 1239-46; O'Nuallain, B., et al., Amyloid and Amyloidosis: Proceedings of the Xth International Symposium on Amyloidoses. 2005. Tours, France: CRC Press; Solomon, A., D. T. Weiss, and J. S. Wall, Clin Cancer Res, 2003. 9(10 Pt 2): p. 3831S-8S). A first-in-human biodistribution study (NCT01409148) was conducted that showed radioiodinated 11-1F4 accumulation specifically in certain amyloid-laden organs in AL patients by PET/CT imaging (FIG. 4D; Wall, J. S., et al., Blood, 2010. 116(13): p. 2241-4).

These data indicated that 11-1F4 was capable of specific targeting of AL amyloid in patients and that, in mice, binding of the mAb could facilitate Mφ-mediated dissolution of human amyloid. The amino acid sequence of the murine 1-1F4 heavy and light variable domains (the Fv region) was determined and used to generate a chimeric reagent (using human IgG1). The c11-1F4 has now undergone Phase 1 clinical evaluation for safety and efficacy in patients with AL amyloidosis (NCT02245867). Interim data indicated that treatment with c11-1F4 mAb was well tolerated and caused improvement in cardiac and renal-related biomarkers in patients with AL amyloidosis (Edwards, C. V., et al., Amyloid, 2017. 24(supl): p. 58-59).

Amyloid-Targeting Peptides

Over the last 7 years, a battery of synthetic amyloid-reactive peptides have been generated and characterized, principally to serve as novel imaging agents for systemic amyloidosis (Martin, E. B., et al., Sci Rep, 2016. 6: p. 22695; Wall, J. S., et al., Molecules, 2015. 20(5): p. 765742; Martin, E. B., et al., Peptides, 2014. 60: p. 63-70; Wall, J. S., et al., Mol Imaging, 2017. 16: p. 1536012117708705; Wall, J. S., et al., Mol Imaging Biol, 2012. 14(4): p. 402-7). Initially, a peptide designated p5 was identified: a 31-amino acid, non-natural, polybasic (+8) reagent that was capable of binding synthetic AL-amyloid fibrils as well as human AL and ATTR amyloid extracts (Martin. E. B., et al., Biochem Biophys Res Commun, 2013. 436(1): p. 85-9; Wall, J. S., et al., Proc Natl Acad Sci USA, 2011. 108(34): p. E586-94). Subsequently, a p5 derivative with an increased net positive charge (peptide p5+14) was generated, which similarly bound AL- and ATTR-associated amyloid but with greater affinity and avidity (Martin, E. B., et al., Sci Rep, 2016. 6: p. 22695; Wall, J. S., et al., Molecules, 2015. 20(5): p. 7657-82).

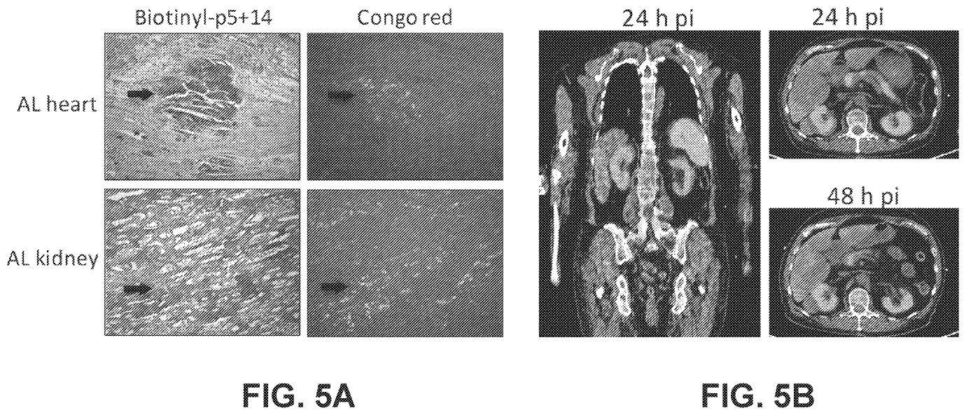

Given the enhanced properties of p5+14, this peptide has been characterized extensively with the goal of translating a radiolabeled variant to the clinic as a novel agent for imaging systemic amyloidosis. Molecular dynamic simulations using an exemplar fibril structure, Aβ (PDB #1BEG), as the substrate indicated that p5+14-fibril interactions are dominated by multivalent electrostatic interactions involving the 12 lysine amino acid sidechains, which can interact with the repeating protein subunits of the amyloid fibril. Additionally the lysine residues can bind the hypersulfated heparan sulfate glycosaminoglycans that are ubiquitous in amyloid deposits (Wall, J. S., et al., Molecules, 2015. 20(5): p. 7657-42). When biotinylated, peptide p5+14 specifically bound human AL amyloid deposits in formalin-fixed paraffin-embedded tissue sections, which were evidenced by green-gold birefringence in Congo red-stained tissue sections (FIG. 5A).

Based on the favorable amyloid-binding and imaging capabilities of p5+14 (i.e. specific binding to amyloid and no reactivity with healthy organs and tissues), as well as an excellent preclinical safety profile, the US FDA approved an Investigational New Drug application (IND #132282) to conduct a Phase 1 PET/CT imaging trial of iodine-¹²⁴-labeled p5+14 peptide in patients with systemic amyloidosis (NCT03678259). The first-in-human imaging study began at the University of Tennessee Medical Center in November 2018 to evaluate the safety and amyloid reactivity of ¹²⁴I-p5+14 peptide. Preliminary data from a patient with AL amyloidosis suggested that the peptide localized to organs likely to contain amyloid in this subject (FIG. 5B).

Following the successful translation of peptide p5+14 into clinical evaluation as an imaging agent for amyloidosis, it was hypothesized that this peptide (or other p5 derivatives)(Wall, J. S., et al., Proc Natl Acad Sci USA, 2018. 115(46): p. E10839-E10848; Wall, J. S., et al., Mol Imaging Biol, 2017. 19(5): p. 714-722; Martin, E. B., et al., J Trans Med, 2017. 15(1): p. 247) could be further exploited as a component of novel amyloid therapeutics.

Accordingly, as described herein the peptide is used as a binding-receptor component of a CAR, which specifically targets and activates Mφ in the presence of amyloid, resulting in phagocytosis.

### Example 2. CAR Design for Amyloid Phagocytosis

The following example describes the design of CAR constructs made up of amyloid binding regions.

In one iteration of the CAR-P cells, usage of the amyloid binding mAb 11-1F4 presented as a single chain variable fragment (scFv) is proposed as the amyloid-binding receptor, since scFv structures have been shown to be active in the CAR setting (Morrissey, M. A., et al., Elife, 2018. 7). An imaging trial of ¹²⁴I-11-1F4 indicated that the mAb imaged only ˜60% of AL patients (Wall, J. S., et al., Blood, 2010. 116(13): p. 2241-4). Therefore, to create a more general CAR construct, a second CAR-P design employs the use of multi-amyloid-reactive peptides as the binding receptor (see below). This is a novel approach, since most CAR constructs use mAb-related scFv as the receptor. Given the increasing utility of tumor (Le Joncour, V. and P. Laakkonen, Bioorg Med Chem, 2018. 26(10): p. 2797-2806) and amyloid targeting peptides (Wall, J. S., et al., Molecules, 2015. 20(5): p. 765742; Wall, J. S., et al., Proc Natl Acad Sci USA, 2018. 115(46): p. E10839-E10848; Wall, J. S., et al., PLoS One, 2013. 8(6): p. e66181; Wall, J. S., et al., Mol Imaging Biol, 2017. 19(5): p. 714-722), it is believed that this approach may open new avenues for the construction of CAR-expressing cells, and provide meaningful benefit to patients by enhancing quality of life and prolonging patient survival.

Six constructs have been designed for initial studies. Three CARs incorporated the 11-1F4 scFv as the amyloid-binding receptor, and the cytoplasmic domain will comprise either: (i) the high affinity murine FcR common γ subunit; (ii) murine CD19+FcR (in tandem), or, (iii) no signal transduction domain (which will serve as a negative control) (FIG. 6, diagram (i)). By way of example, the amino acids that comprise the modules of the 11-1F4 scFv CAR with tandem (CD19+FcR) cytoplasmic domains (designated 11-1F4CAR_{tandem}) are shown in Table 1. The transmembrane and cytoplasmic domains were the same as those used in the previous CAR-P Mφ study (Morrissey, M. A., et al., Elife, 2018. 7).

TABLE 1
Primary structure (single letter amino
acid code) of ^{11-1F4}CAR_{tandem}
components.

| | ^{11-1F4}CAR_{tandem} | | |
|---|---|---|---|
| | | SEQ | |
| | Primary structure | ID | |
| Domain | (amino acids) | NO | Notes |
| Leader | MASPLTRFLS LNLLLLGESI | 28 | Residues 1-27 mouse |
| | ILGSGEA | | CD8 a chain |
| | | | (UniProtKB -P01731 |
| | | | [CD8A_MOUSE]) |
| 11-1F4 VL | DIVLTQSPAS LAVSLGQRAT | 19 | 11-1F4, amino acids |
| | ISYRASKSVS TSGYSYMHWN | | 1-111 |
| | QQKPGQPPRL LIYLVSNLES | | |
| | GVPARFSGSG SGTDFTLNIH | | |
| | PVEEEDAATY YCQHIRELTR | | |
| | FGGGTKLEIK R | | |
| scFv linker | GGSSRSSSSGG GGSGGGG | 27 | See Andris-Widhopf, |
| | | | J., etaL, Cold Spring |
| | | | Harb Protoc, 2011. |
| | | | 2011(9). |
| 11-1F4 VH | QVQLKESGPG LVAPSQSLSI | 20 | 11-1F4, amino acids |
| | TCTVSGFSLS SYGVSWVRQP | | 1-111 |
| | PGKGLEWLGV IWGDGSTNYH | | |
| | PNLMSRSLSI SKDISKSQVL | | |
| | FKLNSLQTDD TATYYCVTLD | | |
| | YWGQGTSVTV S | | |
| Spacer- | KVNSTTTKPVL RTPSPVHPTGT | 29 | Residues 148-218 |
| transmembrane | SQPQRPEDCRP RGSVKGTGLDF | | mouse CD8 α chain |
| domain | ACDIYIWAPLA GICVALLLSLI | | (UniProtKB-P01731 |
| | ITLIC | | [CD8A_MOUSE]) |
| Cytoplasmic | AESYENADEE LAQPVGRMMD | 30 | Residues 500-534 |
| domain I | FLSPHGSAWD PSRE | | mouse CD19 |
| | | | (UniProtKB-P25918 |
| | | | [CD19_MOUSE]) |
| Cytoplasmic | GEPQLCYIEDA VLFLYGIVLTL | 31 | Residues 19-86 |
| domain II | LYCRLKIQVRK AAIASREKADA | | mouse Fc ERG |
| | VYTGLNTRSQE TYETLKHEKPP Q | | precursor |
| | | | (UniProtKB-P20491 |
| | | | [FCERG_MOUSE]) |

A second set of three CARs incorporated the p5+14 peptide as the amyloid receptor and will similarly use the three diverse cytoplasmic signal domains (FIG. 6, diagram (ii)). Protein sequences for each module have been identified from protein sequence databases (e.g., UniProt) that are used to generate the CARs.

For CAR constructs with p5+14, a murine CH2 domain (from the ISG2a Fc domain) was included immediately distal (C-terminal) to the peptide to position it away from the cell surface. It is believed that this will be necessary to minimize interactions of the highly positively charged p5+14 peptide with components of the plasma membrane, notably glycosaminoglycans that have a high negative charge density. The murine IgG2a Fc CH2 domain was chosen because an Fc2a-peptide fusion reagent was previously generated in which the amyloid-reactive peptide was fused to the CH2 domain. In this configuration the amyloid-reactive peptide retained its ability to bind AL amyloid and synthetic amyloid-like AL fibrils (Foster, J. S., et al., Front Immunol, 2017. 8: p. 1082). The sequence of the peptide p5+14 and murine Fc2a CH2 are shown in Table 2.

TABLE 2
Primary structure of p5 + 14 and the CH2
domain used in the ^{P5+l4}CAR constructs.

| | ^{P5+14}CAR_{FcϵR} | | |
|---|---|---|---|
| | | SEQ | |
| | Primary structure | ID | |
| Domain | (amino acids) | NO | Notes |
| 5+14- | ISAMQVTPTV GGGYSKAQKA | 32 | See Wall, J.S., |
| spacer | QAKQAKQAQK AQKAQAKQAK | | et al., |
| | QAQKAQKAQA KQAKQVTPTV | | Molecules, 2015. |
| | | | 20(5): p. 7657-82. |
| CH2 | PNLLGGPSVF IFPPKIKDVL | 33 | From pFUSE- |
| | MISLSPIVTC VVVDVSEDDP | | mIgG2A-Fc2 |
| | DVQISWFVNN VEVHTAQTQT | | expression vector |
| | HREDYNSTLR VVSALPIQHQ | | (Invivogen, San |
| | DWMSGKEFKC KVNNKDLPAP | | Diego, CA) |
| | IERTISKPK | | |

It is noteworthy that the CH2 domain could be substituted by any other compatible sequence to achieve the same goal. Similarly, numerous cytoplasmic phagocytosis signaling domains may be employed: Mφ have four canonical phagocytosis receptors with distinct cytoplasmic elements, although other receptors can be engaged (Taylor, P. R., et al., Annu Rev Immunol, 2005. 23: p. 901-44). In general, signal transduction through immunoreceptor tyrosine-based activation motif (ITAM) elements is considered a major component of the phagocytosis process, and these elements can be used alone, in combination with other elements (Hamerman, J. A., et al., Immunol Rev, 2009. 232(1): p. 42-58; Taylor, P. R., et al., Annu Rev Immunol, 2005. 23: p. 901-44). It is also noteworthy that co-stimulation of the Mφ and induction of phagocytosis may occur through interactions of the surface bound pattern recognition Toll-Like Receptor 2, which has been shown to bind amyloid fibrils (Friedland, R. P., J Alzheimers Dis, 2015. 45(2): p. 349-62; Tukel, C., et al., Cell Host Microbe, 2009. 6(1): p. 45-53).

### Example 3. Expression of CARs in Macrophage Cell Lines

The following example describes the expression of the CAR constructs described in Example 2, above, in macrophage cell lines.

cDNA sequences encoding the entire CAR are synthesized by Genscript (Piscataway, N.J.), cloned into the pEF-ENTR A (Addgene) vector, and recombined into pLenti CMV GFP Dest (Addgene) for packaging as lentivirus by transfection into HEK293T cells using 3^{rd} generation packaging systems with VSV-G psuedotyping. This bicistronic destination vector includes a green fluorescent protein (GFP) sequence to allow identification of positively transfected cells. The murine phagocytic Mφ cell line RAW264.7 (RAW; ATCC TIB-71) is transduced with lentivirus for stable incorporation followed by limited dilution cloning. In addition to the RAW cell line, the non-phagocytic murine monocyte cell line WEHI-274.1 is transduced (WEHI; ATCC CRL-1679). It is reasoned that, since murine RAW Mφ, even those lacking CARs, will exhibit a basal level of amyloid phagocytosis, the WEHI-274 cells serve as a negative control cell to study binding of amyloid to the CARs in the absence of phagocytosis. As a final control, GFP-expressing RAW and WEHI cells are generated by transfection or transduction with pLenti CMV GFP Dest or another vector with only GFP. Surface expression of the 11-1F4 scFv or p5+14 peptide CARs is verified by standard immunofluorescence (Alexa-594) on fixed cells with 11-1F4 anti-idiotype or p5+14-reactive mAbs generated previously (Wall, J. S., et al., Pharm Pat Anal, 2017. 6(5): p. 215-223). The number of expressed receptors per cell is assessed by either cell surface flow cytometry (Qu, C. X., et al., J Clin Lab Anal, 2006. 20(6): p. 250-4) or a radiolabeled antibody binding assay (Dahle, J., et al., Nucl Med Commun, 2007. 28(9): p. 742-7) using 11-1F4 anti-idiotype and anti-peptide p5+14 antibodies.

### Example 4. Binding of Synthetic AL Fibrils and Amyloid Extracts to CAR-P Mφ

As a first step in functional studies, RAW and WEHI cells are transduced, evidenced by the expression of GFP-associated green fluorescence, and cloned by limiting dilution, to study the cell surface binding of synthetic AL-associated amyloid fibrils (Wall, J., et al., Biochemistry, 1999. 38(42): p. 14101-8) and human AL amyloid extracts. The amyloid is labeled using succinimidyl-AlexaFluor-594 (fluorescence emission max=˜610 nm; ThermoFisher) and incubated with CAR-P Mφ for 1-3 hours after which cells associated with amyloid material are quantified by flow cytometry. Data is acquired using an Attune NxT acoustic focusing cytoneter (Applied Biosystems, Carlsbad, Calif.) by gating first for intact cells using forward and side scatter parameters, before gating on GFP (using an excitation at 488 nm and the emission detected with a 532 nm filter). The number of GFP-positive cells also positive for AlexaFluor-594 (574 nm filter) serves as a metric of binding efficiency. This study assesses the integrity of the surface-expressed 11-1F4 scFv and p5+14 peptide and their ability to bind amyloid. RAW and WEHI cells lacking CAR-expression serve as the control populations. Data from n=5 experiments using CAR-P-positive and control cells are compared by using an unpaired T-test or a non-parametric equivalent if the data is not distributed normally.

### Example 5. Ex Vivo Phagocytosis of AL Amyloid

Phagocytosis of synthetic amyloid fibrils and human AL amyloid extracts is studied initially by fluorescence microscopy using amyloid material that has been labeled with the pH-sensitive fluorophore pHrodo red (emission maximum=˜600 nm). This reagent has been used extensively to study the uptake of bacteria and, more recently, amyloid fibrils (Richey et al. (2019) Am J. Pathol. accepted) into the acidified phagolysosome of Mφ where the fluorescence emission of the fluorophore is greatly enhanced (Miksa, M., et al., J Immunol Methods, 2009. 342(1-2): p. 71-7). CAR-expressing RAW cells are grown and seeded at 5×10⁵ cells per well in RPMI medium, in a 24-well culture dish, until they are semi-confluent. Fluorophore-conjugated AL amyloid is added to the wells to a final concentration of 25-50 μg/mL, in a 1-mL final volume. It is anticipated that the amyloid material is insoluble and rapidly settles to the base of the well. After a 2-24 hour incubation period the wells are washed with warmed RPMI and prepared for analysis. Dual fluorophore (red and green) photomicrographs are acquired using a Keyence Bz-x700 fluorescent microscope (40× objective with 3× digital zoom). The number of CAR-P-positive (green) cells containing red-fluorescent amyloid particles is quantified and compared to CAR-P-negative cells using an unpaired, two-tailed t-test, or a non-parametric equivalent.

In parallel, flow cytometric analyses (as described in Example 3, above) is performed using cells in suspension lifted from the culture dishes. The number of coincident red (pHrodo red) and green (GFP-positive) cells is quantified and compared to CAR-negative, GFP-expressing control RAW and WEHI cells.

### Example 6. In Vivo Characterization of Amyloid-CAR-P Mφ in AL Amyloid-Bearing Mice

Human AL amyloid extract is labeled with the near infra-red dye Dylight800-NHS ester (emission maximum=794 nm; Life Technologies) and pHrodo red for optical imaging (iBox Scientia, Analytik Jena). Localized AL masses are induced in male and female immunocompromised (NU/NU) mice (n=5 per cohort) by IP injection of ˜2-20 mg of fluorophore-conjugated amyloid. At one week post-injection, the mice receive an IP injection of 1×10⁶ GFP-positive CAR-P RAW or CAR-P WEHI Mφ (or, as a control, GFP-positive, CAR-negative cells) in a volume of 500 μL sterile PBS. Co-localization of the injected Mφ with the amyloid and phagocytosis is visualized by optical imaging of anesthetized (1.5% isoflurane) mice at 1, 3, 4, and 7 days post injection (Wall, J. S., et al., Proc Natl Acad Sci USA, 2018. 115(46): p. E10839-E10848). The fluorescent intensity of each fluorophore (GFP, pHrodo red, and Dylight800) is quantified from digital images. At the end of the amyloid-targeting experiment, the residual amyloid material, as well as the liver, spleen, lung, and kidneys is harvested post-mortem, fixed in formalin, and tissue sections are prepared and evaluated by fluorescence microscopy to discern the overall biodistribution of the GFP-positive Mφ. The presence of Mφ is quantified in a minimum of 5 consecutive 6 μm-thick tissue sections from morphometric analysis of fluorescence photomicrographs. Data from the mice receiving the CAR-P Mφ is compared with control cells using an unpaired two-tailed t-test.

### Example 7. Exemplary CAR Constructs

Table 3, below, provides exemplary spacer, transmembrane, cytoplasmic region. CH2, leader, p5, and full-length CAR-P amino acid sequences.

TABLE 3
CAR construct amino acid sequences.

```
Spacer/Transmembrane
Mouse (SEQ ID NO: 29)
150
KVN
                    160 170
                        190 200
STTTKPVLRT PSPVHPTGTS QPQRPEDCRP RGSVKGTGLD FACDIYIWAP
                    210
LAGICVALLL SLIITLIC
Human (SEQ ID NO: 57)
                                                                  140
150
TTT PAPRPPTPAP
                    160 170 180
190 200
TIASQPLSLR PEACRPAAGG AVHTRGLDFA CDIYIWAPLA GTCGVLLLSL VITLYC
Cytoplasmic region I (PI3K recruitment) (SEQ ID NO: 42)
500 510 520
530
    L YAAPQLHSIQ SGPSHEEDAD SYENMDKSDD LEPA
Cytoplasmic region II (FcERG) (underlined portion: SEQ ID NO: 41; full-length
sequence: SEQ ID NO: 45)
                    10 20
30 40 50
MISAVILFLL LLVEQAAA LG EPQLCYILDA VLFLYGIVLT LLYCRLKIQV
                          60 70
80
RKAAIASREK ADAVYTGLNT RSQETYETLK HEKPPQ
Mouse CH2 (Fuse) (SEQ ID NO: 33)
PNLLGGPSVF IFPPKIKDVL MISLSPIVTC VVVDVSEDDP DVQISWFVNN
VEVHTAQTQT HREDYNSTLR VVSALPIQHQ DWMSGKEFKC KVNNKDLPAP
IERTISKPK
Leader (SEQ ID NO: 38; bolded), P5 (SEQ ID NO: 39; italicized), Cytoplasmic
domain I (SEQ ID NO: 30, underlined). Cytoplasmic region 1 (SEQ ID NO: 42, bolded
and italicized); full-length sequence: SEQ ID NO: 56
      MALPVTALLL PLALLLHAAR P**SQFR VSP
      TV GGGYSKAQKAQAKQAKQAQKAQKAQAKQAKQ VTPTV
                            410 420
430 440 450
AYEEPDSEEG SEFYENDSNL GQDQVSQDGS GYENPEDEPM GPEEEDSFSN
                      460 470
480 490 500

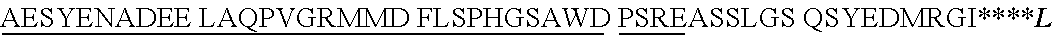

                        510 520
530 540 547

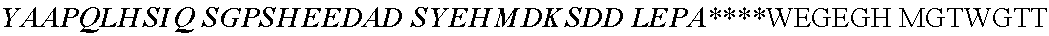

Design Parameters (CAR-P)
Signal peptide: aa 1-21 CD8 (Uniprot Q96QR6_HUMAN)
Extracellular antibody sequence:
V-L chain: aa 23-130 anti-CD19 CAR (Genbank AMZ04819) -
GS linker: ggtggcggtggctcgggcggtggtgggtcgggtggcggcggatct (SEQ ID NO: 46 -
V-H chain: aa 148-267 anti-CD19 CAR (Genbank AMZ04819)
Stalk/Transmembrane: aa 138-206 CDS (Uniprot Q96QR6_HUMAN)
Cytosolic sequence: aa 500-534 Mouse CD19 (Uniprot CD19_MOUSE) fused to aa 19-
86 Mouse Fc ERG precursor (FCERG_MOUSE)
Fluorophore: mGFP
Final CAR-P Construct-345aa (full-length sequence: SEQ ID NO: 43)
MALPVTALLLPLALLLHAARPSQFRVSPTVGGGYSKAQKAQAKQAKQAQKAQKAQAKQAK
QVTPTVPNLLGGPSVFIFPPKIKDVLMISLSPIVTCVVVDVSEDDPDVQISWFVNNVEVH

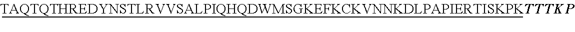

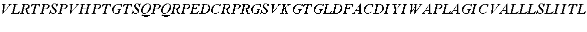

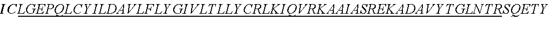

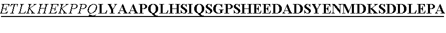




Bold = secretory leader (SEQ ID NO: 38)
Italicized = amyloid binding peptide and spacer (P5) (SEQ ID NO: 39)
Underlined = Ig CH2 domain (SEQ ID NO: 33)
Bolded and italicized = Spacer and transmembrane domain (SEQ ID NO: 40)
Italicized and underlined = Cytoplasmic region II (SEQ ID NO: 41)
Bolded and underlined - Cytoplasmic region I (SEQ ID NO: 42)
```

SEQUENCES
All polypeptide sequences are depicted in the
N-terminal to C-terminal direction.
11-1F4 VL
(SEQ ID NO: 19)
DIVLTQSPAS LAVSLGQRAT ISYRASKSVS TSGYSYMHWN QQKPGQPPRL LIYLVSNLES
GVPARFSGSG SGTDFTLNIH PVEEEDAATY YCQHIRELTR FQGGTKLEIK R
11-1F4 VH
(SEQ ID NO: 20)
QVQLKESGPG LVAPSQSLSI TCTVSGFSLS SYGVSWVRQP PGKGLEWLGV IWGDGSTNYH
PNIMSRSLSI SKDISKSQVL FKLNSLQTDD TATYYCVTLD YWGQGTSVTV S
11-1F4 CDR-H1
(SEQ ID NO: 21)
GFSLSSYGVS
11-1F4 CDR-H2
(SEQ ID NO: 22)
VIWGDGSTNYHPNLMS
11-1F4 CDR-H3
(SEQ ID NO: 23)
LDY
11-1F4 CDR-L1
(SEQ ID NO: 24)
RSSQSLVHRNGNTYLH
11-1F4 CDR-L2
(SEQ ID NO: 25)
KVSNRFS
11-1F4 CDR-L3
(SEQ ID NO: 26)
FQTTYVPNT
scFv linker
(SEQ ID NO: 27)
GGSSRSSSSGG GGSGGGG
CAR leader sequence
(SEQ ID NO: 28)
MASPLTRFLS LNLLLLGESIILGSGEA
Spacer-TM domain
(SEQ ID NO: 29)
KVNSTTTKPVL RTPSPVHPTGT SQPQRPEDCRP RGSVKGTGLDF ACDIYIWAPLA
GICVALLLSLI ITLIC
Cytoplasmic domain I
(SEQ ID NO: 30)
AESYENADEE LAQPVGRMMD FLSPHGSAWD PSRE
Cytoplasmic domain II
(SEQ ID NO: 31)
GEPQLCYILDA VLFLYGIVLTL LYCRLKIQVRK AAIASREKADA VYTGLNTRSQE
TYETLKHEKPP Q
p5+14-spacer
(SEQ ID NO: 32)
ISAMQVTPTV GGGYSKAQKA QAKQAKQAQK AQK AQAKQAK QAQKAQKAQA
KQAKQVTPTV
CH2
(SEQ ID NO: 33)
PNLLGGPSVF IFPPKIKDVL MISLSPIVTC VVVDVSEDDP DVQISWFVNN VEVHTAQTQT
HREDYNSTLRVVSALPIQHQ DWMSGKEFKC KVNNKDLPAP IERTISKPK
11-1F4 VL sequence variant
(SEQ ID NO: 34)
DVVMTQTPLSLPVSLGDQASISCRSSQSLVHRNGNTYLHWYLQKPGQSPKLLIYKVSNRFSG
VPDRFSGSGSGTDFTLKISRVEAEDLGLYFCFQTTYVPNTFGGGTKLEIK
11-1F4 VH sequence variant
(SEQ ID NO: 35)
QVQLKESGPGLVAPSQSLSITCTVSGFSLSSYGVSWVRQPPGKGLEWLGVIWGDGSTNYHPN
LMSRLSISKDISKSQVLFKLNSLQTDDTATYYCVTLDYWGQGTSVTVSS
Spacer
(SEQ ID NO: 36)
VTPTV
p5+14-spacer + CH2
(SEQ ID NO: 37)
ISAMQVTPTV GGGYSKAQKA QAKQAKQAQK AQKAQAKQAK QAQKAQKAQA
KQAKQVTPTV PNLLGGPSVF IFPPKIKDVL MISLSPIVTC VVVDVSEDDP DVQISWFVNN
VEVHTAQTQT HREDYNSTLR VVSALPIQHQ DWMSGKEFKC KVNNKDLPAP IERTISKPK
Secretory leader from “Final CAR-P Construct”
(SEQ ID NO: 38)
MALPVTALLLPLALLLHAARPSQFRVSP
amyloid binding peptide and spacer
(P5) from
“Final CAR-P Construct”
(SEQ ID NO: 39)
TVGGGYSKAQKAQAKQAKQAQKAQKAQAKQAKQVTPTV
Spacer and transmembrane domain from “Final CAR-P Construct”
(SEQ ID NO: 40)
TTTKPVLRTPSPVHPTGTSQPQRPEDCRPRGSVKGTGLDFACDIYIWAPLAGICVALLLSLIITL
IC
Cytoplasmic region II, from “Final CAR-P Construct”
(SEQ ID NO: 41)
LGEPQLCYILDAVLFLYGIVLTLLYCRLKIQVRKAAIASREKADAVYTGLNTRSQETYETLKH
EKPPQ
Cytoplasmic region I, from “Final CAR-P Construct”
(SEQ ID NO: 42)
LYAAPQLHSIQSGPSHEEDADSYENMDKSDDLEPA
Final CAR-P Construct-345aa
(SEQ ID NO: 43)
MALPVTALLLPLALLLHAARPSQFRVSPTVGGGYSKAQKAQAKQAKQAQKAQKAQAKQAK
QVTPTVPNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKTTTKPVLRTPSP
VHPTGTSQPQRPEDCRPRGSVKGTGLDFACDIYIWAPIAGICVALLLSLIITLICLGEPQLCYIL
DAVLFLYGIVLTLLYCRLKIQVRKAAIASREKADAVYTGLNTRSQETYETLKHEKPPQLYAA
PQLHSIQSGPSHEEDADSYENMDKSDDLEPA
Secretory leader+p5+14-spacer + CH2 from
″Final CAR-P Construct″
(SEQ ID NO: 44)
MALPVTALLLPLALLLHAARPSQFRVSPTVGGGYSKAQKAQAKQAKQAQKAQKAQAKQAK
QVTPTVPNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPK
Cytoplasmic region 11
(FcERG), full-length, see Table 3
(SEQ ID NO: 45)
MISAVILFLL LLVEQAAA LG EPQLCYILDA VLFLYGIVLT LLYCRLKIQV RKAAIASREK
ADAVYTGLNT RSQETYETLK HEKPPQ
GS linker from Design Parameters, see Table 3
(SEQ ID NO: 46)
GGTGGCGGTGGCTCGGGCGGTOGTGGGTCGGGTGGCGGCGGATCT
scFv sequence, combination of 11-1F4 VL, scFv linker,
and 11-1F4 VH as shown in Table 1
(SEQ ID NO: 47)
DIVLTQSPAS LAVSLGQRAT ISYRASKSVS TSGYSYMHWN QQKPGQPPRL LIYLVSNLES
GVPARFSGSG SGTDFTLNIH PVEEEDAATY YCQHIRELTR FQGGTKLEIK R GGSSRSSSSGG
GGSGGGG QVQLKESGPG LVAPSQSLSITCTVSGFSLS SYGVSWVRQP PGKGLEWLGV
IWGDGSTNYH PNLMSRSLSI SKDISKSQVL FKLNSLQTDD TATYYCVTLD YWGQGTSVTV
S
scFv sequence with N-terminal leader, combination of
leader, 11-1F4 VL, scFv linker, and 11-
1F4 VH as shown in Table 1
(SEQ ID NO: 48)
MASPLTRFLS LNLLLLGESIILGSGEA DIVLTQSPAS LAVSLGQRAT ISYRASKSVS
TSGYSYMHWN QQKPGQPPRL LIYLVSNLES GVPARFSGSG SGTDFTLNIH PVEEEDAATY
YCQHIRELTR FGGGTKLEIK R GGSSRSSSSGG GGSGGGG QVQLKESGPG LVAPSQSLSI
TCTVSGFSLS SYGVSWVRQP PGKGLEWLGV IWGDGSTNYH PNLMSRSLSI SKDISKSQVL
FKLNSLQTDD TATYYCVTLD YWGQGTSVTV S
Full-length 11-1F4CARtandem, combination of sequences
from Table 1, Cytoplasmic domain 11
only
(SEQ ID NO: 49)
MASPLTRFLSLNLLLLGESIILGSGEADIVLTQSPASLAVSLGQRATISYRASKSVSTSGYSYM
HWNQQKPGQPPRLLIYLVSNLESGVPARFSGSGSGTDFTLNIHPVEEEDAATYYCQHIRELTRF
GGGTKLEIKRGGSSRSSSSGGGGSGGGGQVQLKESGPGLVAPSQSLSITCTVSGFSLSSYGVS
WVRQPPGKGLEWLGVIWGDGSTNYHPNLMSRSLSISKDISKSQVLFKLNSLQTDDTATYYCV
TLDYWGQGTSVTVSKVNSTTTKPVLRTPSPVHPTGTSQPQRPEDCRPRGSVKGTGLDFACDI
YIWAPLAGICVALLLSLIITLICGEPQLCYILDAVLFLYGIVLTLLYCRLKIQVRKAAIASR
EKADAVYTGLNTRSQETYETLKHEKPPQ
Full-length 11-1F4CARtandem, combination of sequences
from Table 1, Cytoplasmic domain I
and II
(SEQ ID NO: 50)
MASPLTRFLSLNLLLLGESIILGSGEADIVLTQSPASLAVSLGQRATISYRASKSVSTSGYSYM
HWNQQKPGQPPRLLIYLVSNLESGVPARFSGSGSGTDFTLNIHPVEEEDAATYYCQHIRELTRF
GGGTKLEIKRGGSSRSSSSGGGGSGGGGQVQLKESGPGLVAPSQSLSITCTVSGFSLSSYGVS
WVRQPPGKGLEWLGVIWGDGSTNYHPNLMSRSLSISKD1SKSQVLFKLNSLQTDDTATYYCV
TLDYWGQGTSVTVSKVNSTTTKPVLRTPSPVHPTGTSQPQRPEDCRPRGSVKGTGLDFACDIY
IWAPLAGICVALLLSLIITLICGEPQLCYILDAVLFLYGIVLTLLYCRLKIQVRKAAIASREK
ADAVYTGLNTRSQETYETLKHEKPPQAESYENADEELAQPVGRMMDFLSPHGSAWDPSRE
Final CAR-P Construct, Cytoplasmic domain II only,
Based on Table 3
(SEQ ID NO: 51)
MALPVTALLLPLALLLHAARPSQFRVSPTVGGGYSKAQKAQAKQAKQAQKAQKAQAKQAKQV
TPTVPNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKTTTKPVLRTPS
PVHPTGTSQPQRPEDCRPRGSVKGTGLDFACDIYIWAPLAGICVALLLSLIITLICLGEPQL
CYILDAVLFLYGIVLTLLYCRLKIQVRKAAIASREKADAVYTGLNTRSQETYETLKHEKPPQ
Final CAR-P Construct-345 a a without the secretory
leader sequence, Based on Table 3
(SEQ ID NO: 52)
TVGGGYSKAQKAQAKQAKQAQKAQKAQAKQAKQVTPTVPNLLGGPSVFIFPPKIKDVLMIS
LSPIVTCVVVDVSEDDPDVQISWFVNNVEVHTAQTQTHREDYNSTLRVVSALPIQHQDWMS
GKEFKCKVNNKDLPAPIERTISKPKTTTKPVLRTPSPVHPTGTSQPQRPEDCRPRGSVKGT
GLDFACDIYIWAPLAGICVALLLSLIITLICLGEPQLCYILDAVLFLYGIVLTLLYCRLKI
QVRKAAIASREKADAVYTGLNTRSQETYETLKHEKPPQLYAAPQLHSIQSGPSHEEDADSY
ENMDKSDDLEPA
Final CAR-P Construct, Cytoplasmic domain II only,
without the secretory leader sequence
(SEQ ID NO: 53)
TVGGGYSKAQKAQAKQAKQAQKAQKAQAKQAKQVTPTVPNLLGGPSVFIFPPKIKDVLMIS
LSPIVTCVVVDVSEDDPDVQISWFVNNVEVHTAQTQTHREDYNSTLRVVSALPIQHQDWMS
GKEFKCKVNNKDLPAPIERTISKPKTTTKPVLRTPSPVHPTGTSQPQRPEDCRPRGSVKGT
GLDFACDIYIWAPLAGICVALLLSLIITLICLGEPQLCYILDAVLFLYGIVLTLLYCRLKI
QVRKAAIASREKADAVYTGINTRSQETYETLKHEKPPQ
Full-length 11-1F4CARtandem, combination of sequences
from Table 1, Cytoplasmic domain 11
only, without secretory lead
(SEQ ID NO: 54)
DIVLTQSPASLAVSLGQRATISYRASKSVSTSGYSYMHWNQQKPGQPPRLLIYLVSNLESGVP
ARFSGSGSGTDFTLNIHPVEEEDAATYYCQHIRELTRFGGGTKLE1KRGGSSRSSSSGGGGSG
GGGQVQLKESGPGLVAPSQSLSITCTVSGFSLSSYGVSWVRQPPGKGLEWLGVIWGDGSTNYH
PNLMSRSLSISKDISKSQVLFKLNSLQTDDTAIYYCVTLDYWGQGTSVTVSKVNSTTTKPVLR
TTSPVHPTGTSQPQRPEDCRPRGSVKGTGLDFACDIYIWAPLAGICVALLLSLIITLICGEPQ
LCYILDAVLFLYGIVLTLLYCRLKIQVRKAAIASREKADAVYTGLNTRSQETYETLKHEKPPQ
Full-length 11-1F4CARtandem, combination of sequences
from Table 1, without secretory lead
(SEQ ID NO: 55)
DIVLTQSPASLAVSLGQRATISYRASKSVSTSGYSYMHWNQQKPGQPPRLLIYLVSNLESGVP
ARFSGSGSGTDFTLNIHPVEEEDAATYYCQHIRELTRFGGGTKLEIKRGGSSRSSSSGGGGSG
GGGQVQLKESGPGLVAPSQSLSITCTVSGFSLSSYGVSWVRQPPGKGLEWLGVIWGDGSTNYH
PNLMSRSLSISKDISKSQVLFKLNSLQTDDTATYYCVTLDYWGQGTSVTVSKVNSTTTKPVLR
TPSPVHPTGTSQPQRPEDCRPRGSVKGTGLDFACDIYIWAPLAGICVALLLSLIITLICGEPQ
LCYILDAVLFLYG1VLTLLYCRLKIQVRKAAIASREKADAVYTGLNTRSQETYETLKHEKPPQ
AESYENADEELAQPVGRMMDFLSPHGSAWDPSRE
Leader, P5, Cytoplasmic domain 1, Cytoplasmic region;
full-length sequence, see Table 3
(SEQ ID NO: 56)
MALPVTALLLPLALLLHAARP**SQFRVSPTVGGGYSKAQKAQAKQAKQAQKAQKAQAKQ
AKQVTPTVAYEEPDSEEGSEFYENDSNLGQDQVSQDGSGYENPEDEPMGPEEEDSFSNAE
SYENADEELAQPVGRMMDFLSPHGSAWDPSREASSLGSQSYEDMRGP****LYAAPQLHS
IQSGPSHEEDADSYENMDKSDDLEPA****WEGEGHMGTWGTT
Spacer/Transmembrane domain, Human, see Table 3
(SEQ ID NO: 57)
TTTPAPRPPTPAPTIASQPLSLRPEACRPAAGGAVHTRGLDFACDIYIWAPLAGTCGVLLLS
LVITLYC
Spacer
(SEQ ID NO: 58)
KVNSTTTKPVLRTPSPVHPTGTSQPQRPEDCRPRGSVKGTGLDFACD
Transmembrane domain
(SEQ ID NO: 59)
IYIWAPLAGICVALLLSLIITLIC

We claim:

1. A chimeric receptor comprising:
a cytoplasmic domain, wherein the cytoplasmic domain comprises a signaling domain of a receptor that when activated activates a macrophage;
a transmembrane domain; and
an extracellular domain, wherein the extracellular domain comprises an amyloid binding region.

2. The chimeric receptor of claim 1, wherein the extracellular domain comprises an antibody or functional fragment thereof.

3. The chimeric receptor of claim 2, wherein the antibody fragment is an scFv.

4. The chimeric receptor of any one of claims 1-3, wherein the antibody comprises a VL comprising a CDRL1, a CDRL2, and an CDRL3 and a VH comprising a CDRH1, a CDRH2, and a CDRH3, wherein the
CDRL1 comprises the amino acid sequence set forth in SEQ ID NO:24;
the CDRL2 comprises the amino acid sequence set forth in SEQ ID NO:25;
the CDRL3 comprises the amino acid sequence set forth in SEQ ID NO:26;
the CDRH1 comprises the amino acid sequence set forth in SEQ ID NO:21;
the CDRH2 comprises the amino acid sequence set forth in SEQ ID NO:22; and
the CDRH3 comprises the amino acid sequence set forth in SEQ ID NO:23.

5. The chimeric receptor of any one of claims 1-4, wherein the antibody comprises a VL comprising the amino acid sequence set forth in SEQ ID NO:19 or 34 and a VH comprising the amino acid sequence set forth in SEQ ID NO:20 or 35.

6. The chimeric receptor of claim 1, wherein the amyloid binding region comprises an 11-1F4 antibody fragment.

7. The chimeric receptor of any one of claims 2-6, wherein the antibody fragment is humanized.

8. The chimeric receptor of claim 1, wherein the extracellular domain comprises an amyloid-reactive peptide.

9. The chimeric receptor of claim 8, wherein the amyloid-reactive peptide comprises the sequence set forth in SEQ ID NO:1-18.

10. The chimeric receptor of claim 8 or 9, wherein the amyloid binding region is joined directly or indirectly to a CH2 domain or fragment thereof.

11. The chimeric receptor of claim 10, wherein the CH2 domain comprises an amino acid sequence having at least 80, 85, 90, 95, 97, 98, or 99% sequence identity to the amino acid sequence set forth in SEQ ID NO:33.

12. The chimeric receptor of any of claims 1-11, wherein the cytoplasmic domain comprises a cytoplasmic domain I, cytoplasmic domain II, or functional fragment thereof.

13. The chimeric receptor of claim 12, wherein the cytoplasmic domain comprises an amino acid sequence having at least 80, 85, 90, 95, 97, 98, or 99% A sequence identity to the amino acid sequence set forth in SEQ ID NO: 30, 31, 41, 42, or 45

14. The chimeric receptor of any of claims 1-12, wherein binding of an amyloid to the extracellular domain activates the cytoplasmic domain of the chimeric receptor.

15. The chimeric receptor of claim 1, wherein the receptor has 80, 85, 90, 95, 97, 98, or 99% sequence identity to the sequence set forth in SEQ ID NO: 43 with or without the secretory leader sequence.

16. The chimeric receptor of claim 1 or the method of any of claims 11-15, wherein each component of the receptor has 80, 85, 90, 95, 97, 98, or 99% sequence identity to the corresponding component of SEQ ID NO:43 together or separately.

17. Nucleic acid encoding the chimeric receptor of any one of claims 1-16.

18. An engineered cell comprising the nucleic acid of claim 17.

19. A method for removing an amyloid, comprising contacting an amyloid deposit with the chimeric receptor of any of claims 1-16 or the engineered cell of claim 18.

20. The method of claim 19, wherein the amyloid is AA, AL, AH, ATTR, Aß2M, Wild type TTR AApoAI, AApoAII, AGel, ALys, ALect2, Afib, ACys, ACal, AMedin, AIAPP, APro, AIns, APrP, or Aβ.

21. The method of claim 20, wherein the amyloid binding region of the chimeric receptor has binding affinity to the amyloid.

22. The method of any of claims 19-21, wherein contacting the amyloid deposit with the chimeric receptor results in at least partial clearance of the amyloid.

23. A method of treating a subject having an amyloid disorder comprising administering to the subject the chimeric receptor of any of claims 1-16 or the engineered cell of claim 18.

24. The method of claim 23, wherein administering to the subject the chimeric receptor comprises administering a macrophage or monocyte expressing the chimeric receptor.
